(19) United States
(12) Patent Application Publication
(10) Pub. No.: US 2025/0090579 A1
(43) Pub. Date: Mar. 20, 2025

(54) DNVSIG3 AND DNVSIG8 RECEPTORS AND METHODS OF USING THE SAME

(71) Applicant: Kite Pharma, Inc., Santa Monica, CA (US)

(72) Inventors: Felipe Bedoya, Santa Monica, CA (US); David Barrett, Santa Monica, CA (US); Vijay Gopal Reddy Peddareddigari, Santa Monica, CA (US)

(73) Assignee: Kite Pharma, Inc., Santa Monica, CA (US)

(21) Appl. No.: 18/292,254

(22) PCT Filed: Jul. 25, 2022
(86) PCT No.: PCT/US2022/038135

§ 371 Date: Jan. 25, 2024

Related U.S. Application Data
(60) Provisional application No. 63/225,840, filed on Jul. 26, 2021.

(51) Int. Cl. A61K 35/17; C07K 14/705; C12N 15/86

(52) U.S. Cl. CPC A61K 35/17; C07K 14/70503; C12N 15/86; C07K 2319/02; C07K 2319/03; C12N 2740/15043

(57) ABSTRACT

Described are dominant-negative receptor mutants of V-Set and Immunoglobulin domain containing 3 (VSIG3) receptors and V-Set and Immunoglobulin domain containing 8 (VSIG8) receptors and methods of making the same for use in applications drawn to modulating the downstream signal transduction effects of VSIG3 and VSIG8 and their corresponding ligands. The invention is drawn to use of the use of the dnVSIG3 and/or dnVSIG8 receptor mutants to mitigate immunosuppressive effects associated with functional forms of VSIG3 and/or VSIG8 in the presence of their cognate ligands.

### CROSS-REFERENCE TO RELATED APPLICATION

This application claims priority to U.S. Provisional Application No. 63/225,840, filed Jul. 26, 2021, the contents of which is specifically incorporated by reference in its entirety

### FIELD

The present disclosure relates to the field of dominant-negative (DN) receptor mutants of V-Set and Immunoglobulin domain containing 3 (VSIG3) receptors and V-Set and Immunoglobulin domain containing 8 (VSIG8) receptors and methods of making the same for use in applications drawn to modulating the downstream signal transduction effects of VSIG3 and VSIG8 and their corresponding ligands. The present disclosure is further drawn to engineered cells comprising the DNVSIG3 and/or DNVSIG8 receptor mutants and methods of administering the cells to a subject in need. More specifically, the present disclosure is concerned with engineered human immune cells comprising the DNVSIG3 and/or DNVSIG8 receptor mutants and further comprising a modified T cell receptor and/or a modified chimeric antigen receptor.

### STATEMENT REGARDING SEQUENCE LISTING

The sequence listing associated with this application is provided in text format in lieu of a paper copy, and is hereby incorporated by reference into the specification. The name of the text file containing the sequence listing is 125400_1235_Sequence_Listing.txt. The text file is ˜102 kb, was created on Jun. 16, 2021, and is being submitted electronically via EFS-Web.

### BACKGROUND

Recent breakthroughs in cancer immunotherapy have demonstrated the power that harnessing the immune system can have to eliminate cancer cells. Most of these therapies rely on the triggering of immune responses upon administering monoclonal antibodies that block various immunosuppressive signals. However, relying on endogenous responses makes it difficult to achieve high therapeutic efficacy while avoiding toxic autoimmune side effects. For more precise therapy, T cells can be engineered with modified antigen receptors, such as chimeric antigen receptors (CARs) to recognize and exert cytotoxic effects on cells expressing specific antigens. A challenge to the success of these therapies is the immunosuppressive microenvironment that redirected T cells encounter upon infiltration into a tumor bed.

To achieve therapeutic success within solid tumors, T cells with modified antigen receptors need to overcome immunosuppressive signals. One approach has been to block immunosuppressive pathways such as PD-1 and/or CTLA-4 in combination with the engineered T cell therapies to achieve an enhanced response. Many cancers, such as prostate cancer, are known to secrete molecules capable of creating an immunosuppressive milieu. Identifying pathways associated with these immunosuppressive molecules provides an opportunity to mitigate the signal transduction that ultimately results in these immunosuppressive effects.

The aim of the present disclosure is to fulfill a need for mitigating the immunosuppressive effects of immunosuppressive molecules on engineered T cells used in cancer immunotherapies. This need is met with the use engineered T cells expressing artificially created dominant-negative forms V-set and immunoglobulin domain containing 3 (VSIG3) and/or V-set and immunoglobulin domain containing 8 (VSIG8) that mitigate or eliminate signal transduction in VSIG3 and/or VSIG8.

### SUMMARY OF THE DISCLOSURE

The present disclosure is generally drawn to dominant-negative VSIG3 and VSIG8 receptors in which the wild-type receptors have been modified such that signal transduction in the receptors is disrupted, as compared to the wild-type receptors. The present disclosure is further drawn to polynucleotides, vectors, cells, and methods of administration associated with the dominant-negative VSIG3 and VSIG8 receptors described herein.

In some aspects, the disclosure is broadly drawn to a modified polypeptide comprising an amino acid sequence sharing at least about 90%, at least about 91%, at least about 92%, at least about 93%, at least about 94%, at least about 95%, at least about 96%, at least about 97%, at least about 98%, at least about 99%, or about 100% sequence identity with any one of SEQ ID NOs: 20-28. In some aspects, the amino acid sequence shares at least about 95% sequence identity with SEQ ID NO:20. In some aspects, the amino acid sequence shares at least about 99% sequence identity with SEQ ID NO:20. In some aspects, the amino acid sequence is SEQ ID NO: 20.

In some aspects, the disclosure is broadly drawn to a modified polypeptide comprising an amino acid sequence sharing at least about 90%, at least about 91%, at least about 92%, at least about 93%, at least about 94%, at least about 95%, at least about 96%, at least about 97%, at least about 98%, at least about 99%, or about 100% sequence identity with any one of SEQ ID NOs: 61-70. In some aspects, the amino acid sequence shares at least about 95% sequence identity with SEQ ID NO:61. In some aspects, the amino acid sequence shares at least about 99% sequence identity with SEQ ID NO:61. In some aspects, the amino acid sequence is SEQ ID NO: 61.

In some aspects, the polynucleotides encoding the polypeptides described herein further comprising a signal peptide sequence. In some aspects, the disclosure is broadly drawn to a polynucleotide sequence encoding any one of the polypeptides described herein. In some aspects, the disclosure is broadly drawn to a polynucleotide sequence comprising a nucleic acid sequence sharing at least about 90%, at least about 91%, at least about 92%, at least about 93%, at least about 94%, at least about 95%, at least about 96%, at least about 97%, at least about 98%, at least about 99%, or about 100% sequence identity with any one of SEQ ID NOs: 50-59. In some aspects, the amino acid sequence shares at least about 95% sequence identity with SEQ ID NO: 50. In some aspects, the amino acid sequence shares at least about 99% sequence identity with SEQ ID NO:50. In some aspects, the nucleic acid sequence is SEQ ID NO:50.

In some aspects, the disclosure is broadly drawn to a polynucleotide sequence comprising a nucleic acid sequence sharing at least about 90%, at least about 91%, at least about 92%, at least about 93%, at least about 94%, at least about 95%, at least about 96%, at least about 97%, at least about 98%, at least about 99%, or about 100% sequence identity with any one of SEQ ID NOs: 72-81. In some aspects, the amino acid sequence shares at least about 95% sequence identity with SEQ ID NO:72. In some aspects, the amino acid sequence shares at least about 99% sequence identity with SEQ ID NO:72. In some aspects, the nucleic acid sequence is SEQ ID NO:72.

In some aspects, the disclosure is broadly drawn to a composition comprising any one or more of the polypeptides described herein.

In some aspects, the disclosure is broadly drawn to a vector comprising any one or more of the polynucleotide sequences sequences described herein that encode any one of the engineered dominant-negative receptors described herein. In some aspects, any one or more of the polynucleotide sequences described herein are operably linked to one or more polypeptides. In some aspects, any one or more of the polynucleotide sequences described herein are operably linked to one or more polypeptides.

In some aspects, the vector is a viral vector. In some aspects, the viral vector is selected from the group consisting of lentivirus vector, gamma retrovirus vector, foamy virus vector, adeno-associated virus vector, adenovirus vector, pox virus vector, herpes virus vector, and an engineered hybrid virus vector. In some aspects, the vector is lentivirus vector.

In some aspects, the disclosure is broadly drawn to a cell comprising any one or more of the polypeptides described herein. In some aspects, the polypeptide is a dominant-negative receptor with disrupted activity as compared to activity of the wild-type VSIG3. In some aspects, the polypeptide is a dominant-negative receptor with disrupted activity as compared to activity of the wild-type VSIG8.

In some aspects, the disclosure is broadly drawn to a cell comprising any one or more of the polynucleotides described herein. In some aspects, the disclosure is broadly drawn to a cell comprising any one or more of the vectors described herein. In some aspects, the cell is selected from the group consisting of bacterial cell, fungal cell, yeast cell, animal cell, and human cell. In some aspects, the cell is a human cell. In some aspects, the human cell is an immune cell. In some aspects, the immune cell is a T cell.

In some aspects, the cell, such as a T cell, comprises a modified antigen receptor. In some aspects, the modified antigen receptor is a T cell receptor or a chimeric antigen receptor (CAR). In some aspects, the dominant-negative receptor is incapable of signal transduction. In some aspects, the extracellular domain of the dominant-negative receptor is capable of binding to its corresponding ligand. In some aspects, the corresponding ligand is VISTA. In some aspects, expression of the wild-type VSIG3 has been downregulated in the cell. In some aspects, expression of the wild-type VSIG8 has been downregulated in the cell. In some aspects, In some aspects, expression of the wild-type VSIG3 and VSIG8 have been downregulated in the cell.

In some aspects, the modified antigen receptor is a CAR and the CAR comprises a binder selected from the group consisting of prostate-specific membrane antigen (PSMA), Tn glycoform of mucin 1 (TnMUC1), mesothelin, glypican 2 (GPC2), fibroblast activation protein (FAP), folate receptor alpha (FRα), epidermal growth factor receptor (EGFR), interleukin-13 receptor subunit alpha 2 (IL-13Ralpha2), and any combination thereof. In some aspects, the modified antigen receptor is a CAR, and the CAR comprises a co-stimulatory domain selected from the group consisting of CD2, 4-1BB, ICOS, and CD27. In some aspects, the modified antigen receptor is a CAR, and the CAR comprises a switch receptor and/or a dominant negative receptor, wherein the receptors are selected from the group consisting of PD1/CD28, PDL1/CD28, CTLA4/CD28, BTLA/CD28, BTLA/ICOS, TIM3/CD28, TIGIT/CD226, dnTGFB, TGFβ/IL-12R, TGFβ/CD28, TGFβ/OX40, IFNγ/CD28, IFNγ/OX40, and IFNγ/IL-12R. In some aspects, the binder comprises a combination of EGFR and IL-13Ralpha2. In some aspects, the modified antigen receptor is a CAR, and the CAR comprises a CD32 signaling domain.

In some aspects, the disclosure is broadly drawn to a method of administering any one or more of the engineered cells described herein that comprise polynucleotides and/or polypeptides corresponding to the engineered dominant-negative VSIG3 and/or VSIG8 receptors, the method comprising administering to a subject a composition comprising the cell. In some aspects, the cell is autologous to the subject. In some aspects, the cell is allogeneic to the subject. In some aspects, the disclosure is broadly drawn to a method of generating a modified cell, the method comprising introducing into a cell any one or more of the vectors described herein.

In some aspects, the cell is selected from the group consisting of: bacterial cell, fungal cell, yeast cell, animal cell, and human cell. In some aspects, the cell is a human immune cell. In some aspects, the immune cell is a T cell. In some aspects, the T cell comprises a modified antigen receptor.

In some aspects, the modified antigen receptor is a T cell receptor (TCR) or a chimeric antigen receptor (CAR). In some aspects, the modified antigen receptor is a CAR and the CAR comprises a binder selected from the group consisting of prostate-specific membrane antigen (PSMA), Tn glycoform of mucin 1 (TnMUC1), mesothelin, glypican 2 (GPC2), fibroblast activation protein (FAP), folate receptor alpha (FRα), and a combination of epidermal growth factor receptor (EGFR) and interleukin-13 receptor subunit alpha 2 (IL-13Ralpha2). In some aspects, the binder comprises a combination of EGFR and IL-13Ralpha2.

In some aspects, the modified antigen receptor is a CAR, and the CAR comprises a co-stimulatory domain selected from the group consisting of CD2, 4-1BB, ICOS, and CD27. In some aspects, the modified antigen receptor is a CAR, and the CAR comprises a switch receptor and/or a dominant negative receptor, wherein the receptors are selected from the group consisting of PD1/CD28, PDL1/CD28, CTLA4/CD28, BTLA/CD28, BTLA/ICOS, TIM3/CD28, TIGIT/CD226, dnTGFβ, TGFβ/IL-12R, TGFβ/CD28, TGFβ/OX40, IFNγ/CD28, IFNγ/OX40, and IFNγ/IL-12R. In some aspects, the modified antigen receptor is a CAR, and the CAR comprises a CD35 signaling domain.

Both the foregoing summary and the following description of the drawings and detailed description are exemplary and explanatory. They are intended to provide further details of the invention, but are not to be construed as limiting. Other objects, advantages, and novel features will be readily apparent to those skilled in the art from the following detailed description of the invention.

### BRIEF DESCRIPTION OF THE DRAWINGS

FIG. 1 depicts a characterization of the wild-type VSIG3 and the engineered dominant-negative VSIG3 (dnVSIG3). Both the VSIG3 and the dnVSIG3 transverses the lipid bi-layer of the cell membrane, and both comprise an extracellular domain and a transmembrane domain. The dnVSIG3 lacks most or all of the intracellular domain and is incapable of downstream signaling, whereas the VSIG3 comprises an intact intracellular domain capable of downstream signaling.

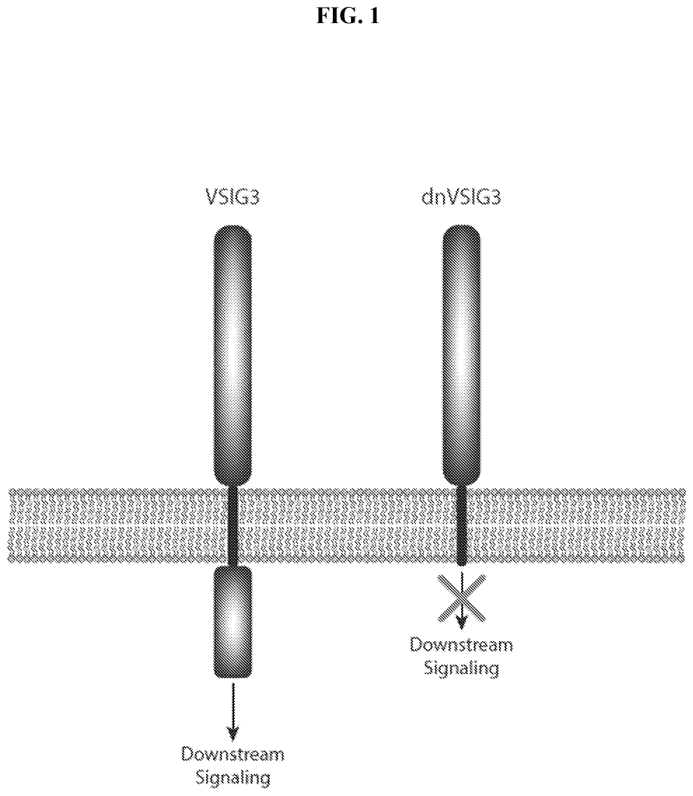

FIG. 2 depicts a characterization of VSIG3 and dnVSIG3, simply demonstrating the relative difference in length of the wild-type VSIG3 versus the truncated dn VSIG3.

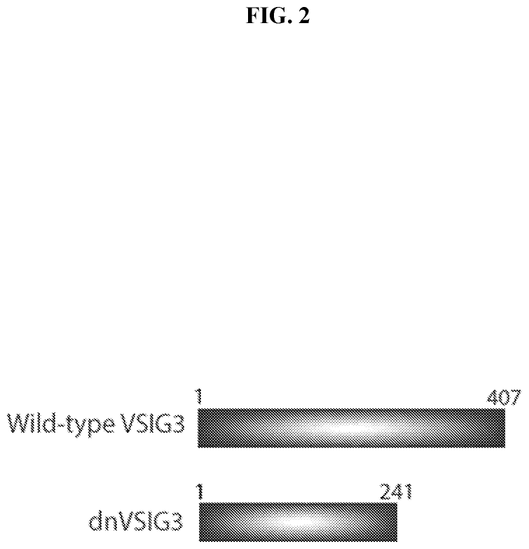

### DETAILED DESCRIPTION OF THE DISCLOSURE

### I. Dominant-Negative Mutants

Dominant-negative mutants represent an important class of mutation in which one or more mutations in a receptor interferes with or disrupts the function of the wild-type version of the receptor, as compared to the unmodified receptor.

In some aspects, the dominant-negative mutant comprises an extracellular domain that is capable of binding to its corresponding ligand. In some aspects, the extracellular domain of the dominant-negative mutant exhibits no difference in affinity for its corresponding ligand.

In some aspects, the interference/disruption of the function of the receptor results in a complete loss of function of the receptor. In some aspects, the extracellular domain(s) of the receptor can bind its corresponding ligand or binding partner, but intracellular signaling cannot occur because of the absence or alteration in a domain of the receptor-thus, the cells expressing this mutated receptor will be unable to respond in the presence of the corresponding receptor ligand, as compared to a wild-type form of the receptor.

In some aspects, the interference/disruption of the function of the receptor results in a reduction in the ability to transduce a signal from the extracellular side of the receptor to the intracellular side of the receptor. In some aspects, the reduction in the ability to transduce a signal from the extracellular side of the receptor to the intracellular side of the receptor is a reduction of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99%.

### II. VSIG3 and VSIG8

The dominant-negative mutant receptors of the present disclosure are drawn to V-set and immunoglobulin domain containing 3 (VSIG3) and V-set and immunoglobulin domain containing 8 (VSIG8).

VSIG3 is known to have at least one binding partner or ligand, which is V domain-containing immunoglobulin suppressor of T-cell activation (VISTA). The VSIG3/VISTA pathway has been described as capable of inhibiting human T cell functions. VSIG3 is associated with inhibition of human T cell proliferation in the presence of T cell receptor signalling and further associated with considerable reductions in cytokine and chemokine production in human T cells, including IFN-γ, IL-2, IL-17, CCL5/Rantes, CCL3/MIP-1α, and CXCL11/I-TAC. See Wang et al., Immunology 156 (1): 74-85 (2019). While Wang has described VSIG3 as a ligand of VISTA, it is believed that VSIG3 is transmembrane receptor comprising an extracellular domain, a transmembrane domain, and an intracellular domain.

The dnVSIG3 receptor, upon expression on T cells, engages VISTA, a checkpoint inhibitor, mainly expressed on tumor and myeloid cells, and prevents the immunosuppressive signalling caused by the VSIG3-VISTA ligation.

A wild-type human VSIG3 amino acid sequence is associated with Uniprot Accession Q5DX21. This amino acid sequence consists of 431 amino acids (SEQ ID NO:1). The corresponding nucleic acid sequence, coding sequence, that encodes the this wild-type human VSIG3 consists of 1,296 nucleotides (SEQ ID NO:31). The amino acid sequence of SEQ ID NO: 1 comprises a signal peptide (positions 1-22), an extracellular domain (positions 23-241), a transmembrane domain (positions 242-262), and an intracellular domain (positions 263-431. The wild-type human VSIG3 has 18 additional variants described as naturally occurring isoforms or splice variants. The VSIG3 sequences as well as the dominant-negative VSIG3 (DNVSIG3) sequences are described in the below table.

In some aspects, the dominant-negative VSIG3 comprises, relative to the wild-type, the following mutations:

(1) a truncation from the N-terminus-side or the C-terminus-side of the intracellular domain of at least about 5 residues, at least about 10 residues, at least about 15 residues, at least about 20 residues, at least about 25 residues, at least about 30 residues, at least about 35 residues, at least about 40 residues, at least about 45 residues, at least about 50 residues, at least about 55 residues, at least about 60 residues, at least about 65 residues, at least about 70 residues, at least about 75 residues, at least about 80 residues, at least about 85 residues, at least about 90 residues, at least about 95 residues, at least about 100 residues, at least about 105 residues, at least about 110 residues, at least about 115 residues, at least about 120 residues, at least about 125 residues, at least about 130 residues, at least about 135 residues, at least about 140 residues, at least about 145 residues, at least about 150 residues, at least about 155 residues, at least about 160 residues, or at least about 165 residues, or any number of residues that falls in-between any of these values, e.g., about 7 residues, about 52 residues, about 93 residues, etc.

(2) a truncation from the N-terminus-side or the C-terminus-side of the intracellular domain of about 5 residues, about 10 residues, about 15 residues, about 20 residues, about 25 residues, about 30 residues, about 35 residues, about 40 residues, about 45 residues, about 50 residues, about 55 residues, about 60 residues, about 65 residues, about 70 residues, about 75 residues, about 80 residues, about 85 residues, about 90 residues, about 95 residues, about 100 residues, about 105 residues, about 110 residues, about 115 residues, about 120 residues, about 125 residues, about 130 residues, about 135 residues, about 140 residues, about 145 residues, about 150 residues, about 155 residues, about 160 residues, or about 165 residues, or any number of residues that falls in-between any of these values, e.g., about 7 residues, about 52 residues, about 93 residues, etc.

(3) a deletion within the intracellular domain of about 5 consecutive residues, about 10 consecutive residues, about 15 consecutive residues, about 20 consecutive residues, about 25 consecutive residues, about 30 consecutive residues, about 35 consecutive residues, about 40 consecutive residues, about 45 consecutive residues, about 50 consecutive residues, about 55 consecutive residues, about 60 consecutive residues, about 65 consecutive residues, about 70 consecutive residues, about 75 consecutive residues, about 80 consecutive residues, about 85 consecutive residues, about 90 consecutive residues, about 95 consecutive residues, about 100 consecutive residues, about 105 consecutive residues, about 110 consecutive residues, about 115 consecutive residues, about 120 consecutive residues, about 125 consecutive residues, about 130 consecutive residues, about 135 consecutive residues, about 140 consecutive residues, about 145 consecutive residues, about 150 consecutive residues, about 155 consecutive residues, about 160 consecutive residues, or about 165 consecutive residues, or any number of residues that falls in-between any of these values, e.g., about 7 residues, about 52 residues, about 93 residues, etc.

(4) a deletion of the entire intracellular domain.

(5) a truncation of the C-terminus of the intracellular domain such that the resulting intracellular domain consists of about 1 to about 30 residues of the N-terminal portion of intracellular domain. In some aspects, the resulting intracellular domain consists of about 5 to about 30 residues, about 10 to about 30 residues, about 15 to about 30 residues, about 20 to about 30 residues, about 25 to about 30 residues, about 5 to about 25 residues, about 10 to about 25 residues, about 15 to about 25 residues, about 20 to about 25 residues, about 5 to about 20 residues, about 10 to about 20 residues, about 15 top about 20 residues, about 5 to about 15 residues, about 10 to about 15 residues, or about 5 to about 10 residues of the N-terminal portion of the intracellular domain.

(6) a deletion of between about 1 to about 10, about 2 to about 10, about 3 to about 10, about 4 to about 10, about 5 to about 10, about 6 to about 10, about 7 to about 10, about 8 to about 10, about 2 to about 8, about 2 to about 6, about 2 to about 5, about 2 to about 4, about 4 to about 6, about 4 to about 5, about 1 to about 5, about 2 to about 3, or about 1 to about 2 residues from the transmembrane domain.

In some aspects, the mutations of any one of (1)-(5) are further combined with the mutation of (6).

In some aspects, any polynucleotide sequence encoding any one of the dn VSIG3 receptors are contemplated. In some aspects, the polynucleotide sequence is codon-optimized. A codon-optimized polynucleotide sequence encodes the same amino acid sequence as a non-codon-optimized polynucleotide sequence.

TABLE 1
Description of Sequences.

| | SEQ ID | | |
|---|---|---|---|
| | NOs for | | |
| SEQ ID | corresponding | | |
| NOS for | nucleic | | |
| amino acid | acid | | |
| sequences | sequences | Name | Comments |
| 1 | 31 | VSIG3 | WT splice variant |
| | | Variant 2, also | |
| | | known as Uniprot | |
| | | Accession Q5DX21 | |
| 2 | 32 | VSIG3 Variant 5 | WT splice variant |
| 3 | 33 | VSIG3 Variant 7 | WT splice variant |
| 4 | 34 | VSIG3 Variant 1 | WT splice variant |
| 5 | 35 | VSIG3 Variant 9 | WT splice variant |
| 6 | 36 | VSIG3 Variant 6 | WT splice variant |
| 7 | 37 | VSIG3 Variant 8 | WT splice variant |
| 8 | 38 | VSIG3 Variant 11 | WT splice variant |
| 9 | 39 | VSIG3 Variant 10 | WT splice variant |
| 10 | 40 | VSIG3 Variant 4 | WT splice variant |
| 11 | 41 | VSIG3 Variant 3 | WT splice variant |
| 12 | 42 | VSIG3 Isoform X1 | WT splice variant |
| 13 | 43 | VSIG3 Isoform X4 | WT splice variant |
| 14 | 44 | VSIG3 Isoform X5 | WT splice variant |
| 15 | 45 | VSIG3 Isoform X2 | WT splice variant |
| 16 | 46 | VSIG3 Isoform X3 | WT splice variant |
| 17 | 47 | VSIG3 Isoform X6 | WT splice variant |
| 18 | 48 | VSIG3 Isoform X8 | WT splice variant |
| 19 | 49 | VSIG3 Isoform X7 | WT splice variant |
| 20 | 50 | DNVSIG3 Mutant 1 | Mutant of VSIG3 |
| | | | Variant 6 |
| 21 | 51 | DNVSIG3 Mutant 2 | Mutant of VSIG3 |
| | | | Variant 6 |
| 22 | 52 | DNVSIG3 Mutant 3 | Mutant of VSIG3 |
| | | | Variant 6 |
| 23 | 53 | DNVSIG3 Mutant 4 | Mutant of VSIG3 |
| | | | Variant 6 |
| 24 | 82 | DNVSIG3 Mutant 5 | Mutant of VSIG3 |
| | | | Variant 6 |
| 25 | 54 | DNVSIG3 Mutant 6 | Mutant of VSIG3 |
| | | | Variant 6 |
| 26 | 55 | DNVSIG3 Mutant 7 | Mutant of VSIG3 |
| | | | Variant 2 |
| 27 | 56 | DNVSIG3 Mutant 8 | Mutant of VSIG3 |
| | | | Variant 2 |
| 28 | 57 | DNVSIG3 Mutant 9 | Mutant of VSIG3 |
| | | | Variant 2 |
| 29 | 58 | DNVSIG3 | Mutant of VSIG3 |
| | | Mutant 10 | Variant 2 |
| 30 | 59 | DNVSIG3 | Mutant of VSIG3 |
| | | Mutant 11 | Variant 2 |
| 60 | 71 | VSIG8 | WT |
| | | Wild-Type, also | |
| | | known as Uniprot | |
| | | Accession PODPA2 | |
| 61 | 72 | DNVSIG8 Mutant 1 | Mutant of VSIG8 |
| | | | Wild-Type |
| 62 | 73 | DNVSIG8 Mutant 2 | Mutant of VSIG8 |
| | | | Wild-Type |
| 63 | 74 | DNVSIG8 Mutant 3 | Mutant of VSIG8 |
| | | | Wild-Type |
| 64 | 75 | DNVSIG8 Mutant 4 | Mutant of VSIG8 |
| | | | Wild-Type |
| 65 | 76 | DNVSIG8 Mutant 5 | Mutant of VSIG8 |
| | | | Wild-Type |
| 66 | 77 | DNVSIG8 Mutant 6 | Mutant of VSIG8 |
| | | | Wild-Type |
| 67 | 78 | DNVSIG8 Mutant 7 | Mutant of VSIG8 |
| | | | Wild-Type |
| 68 | 79 | DNVSIG8 Mutant 8 | Mutant of VSIG8 |
| | | | Wild-Type |
| 69 | 80 | DNVSIG8 | Mutant of VSIG8 |
| | | Mutant 9 | Wild-Type |
| 70 | 81 | DNVSIG8 | Mutant of VSIG8 |
| | | Mutant 10 | Wild-Type |

VSIG8 is described as involved in inhibiting the production of cytokines such as IL-2, IFN-γ, IL-17, IL-6, and IL-19; and chemokines such as MCP-1, MCP-10, and IP-10; and other proteins such as IGFBP3 and RBP4 in anti-CD3 activated human CD3 T cells. It was further described as significantly reducing the production of IFN-γ and IL-2 on both CD4 and CD8 T cells in the presence of T cell receptor signalling; and capable of markedly suppressing anti-CD3-induced human T cell proliferation. VSIG8 is further associated with the profound decrease in the conversion of naïve CD4+ T cells into Th1 cells. See Wang et al., J. Immunol. 200 (1 Supplement) 47.4 (2018). It is believed that VSIG3 is transmembrane receptor comprising an extracellular domain, a transmembrane domain, and an intracellular domain; and a likely binding partner (ligand) with VISTA. The interaction between VISTA and VSIG8/dnVSIG8 is believed to operate in the same manner as described above for VSIG3.

The wild-type human VSIG8 amino acid sequence consists of 414 amino acids (SEQ ID NO: 60). The nucleic acid sequence, coding sequence, that encodes the wild-type human VSIG8 consists of 1,245 nucleotides (SEQ ID NO:71). The amino acid sequence of SEQ ID NO:60 comprises a signal peptide (positions 1-21), an extracellular domain (positions 22-263), a transmembrane domain (positions 264-284), and an intracellular domain (positions 285-414. The wild-type human VSIG8 protein is associated with Uniprot Accession PODPA2.

In some aspects, the dominant-negative VSIG8 comprises, relative to the wild-type, the following mutations:

(1) a truncation from the N-terminus-side or the C-terminus-side of the intracellular domain of at least about 5, at least about 10, at least about 15, at least about 20, at least about 25, at least about 30, at least about 35, at least about 40, at least about 45, at least about 50, at least about 55, at least about 60, at least about 65 residues, at least about 70, at least about 75, at least about 80, at least about 85, at least about 90, at least about 95, at least about 100, at least about 105, at least about 110, at least about 115, at least about 120, at least about 125, or at least about 130 residues, or any number of residues that falls in-between any of these values, e.g., about 7 residues, about 52 residues, about 93 residues, etc.

(2) a truncation from the N-terminus-side or the C-terminus-side of the intracellular domain of about 5, about 10, about 15, about 20, about 25, about 30, about 35, about 40, about 45, about 50, about 55, about 60, about 65, about 70, about 75, about 80, about 85, about 90, about 95, about 100, about 105, about 110, about 115, about 120, about 125, or about 130 residues, or any number of residues that falls in-between any of these values, e.g., about 7 residues, about 52 residues, about 93 residues, etc.

(3) a deletion within the intracellular domain of about 5, about 10, about 15, about 20, about 25, about 30, about 35, about 40, about 45, about 50, about 55, about 60, about 65, about 70, about 75, about 80, about 85, about 90, about 95, about 100, about 105, about 110, about 115, about 120, about 125, or about 130 consecutive residues, or any number of consecutive residues that falls in-between any of these values, e.g., about 7 residues, about 52 residues, about 93 residues, etc.

(4) a deletion of the entire intracellular domain.

(5) a truncation of the C-terminus of the intracellular domain such that the resulting intracellular domain consists of about 1 to about 30 residues of the N-terminal portion of the intracellular domain. In some aspects, the resulting intracellular domain consists of about 5 to about 30 residues, about 10 to about 30 residues, about 15 to about 30 residues, about 20 to about 30 residues, about 25 to about 30 residues, about 5 to about 25 residues, about 10 to about 25 residues, about 15 to about 25 residues, about 20 to about 25 residues, about 5 to about 20 residues, about 10 to about 20 residues, about 15 to about 20 residues, about 5 to about 15 residues, about 10 to about 15 residues, or about 5 to about 10 residues of the N-terminal portion of the intracellular domain.

(6) a deletion of between about 1 to about 10, about 2 to about 10, about 3 to about 10, about 4 to about 10, about 5 to about 10, about 6 to about 10, about 7 to about 10, about 8 to about 10, about 2 to about 8, about 2 to about 6, about 2 to about 5, about 2 to about 4, about 4 to about 6, about 4 to about 5, about 1 to about 5, about 2 to about 3, or about 1 to about 2 residues from the transmembrane domain.

In some aspects, the mutations of any one of (1)-(5) are further combined with the mutation of (6).

The VSIG8 sequences as well as the dominant-negative VSIG8 (DNVSIG8) sequences are described in table 1, above.

In some aspects, any polynucleotide sequence encoding any one of the dn VSIG3 receptors are contemplated. In some aspects, the polynucleotide sequence is codon-optimized. A codon-optimized polynucleotide sequence encodes the same amino acid sequence as a non-codon-optimized polynucleotide sequence.

III. Effects of dn VSIG3 and/or dnVSIG8

In some aspects, a cell expressing dnVSIG3 and/or dnVSIG8 exhibits a reduction in the amount of one or more cytokines produced by the cell when the cell is in the presence of a cognate ligand that binds with the extracellular domain of dnVSIG3 and/or dnVSIG8, as compared to the wild-type form of the corresponding VSIG3 or VSIG8.

In some aspects, the one or more cytokines are interleukins. In some aspects, the interleukins may be chosen from IL-1α, IL-1β, IL-1ra (antagonist), IL-2, IL-3, IL-4, IL-5, IL-6, IL-7, IL-8, IL-9, IL-10; IL-11, IL-12, IL-13, IL14, IL-15, IL-16, IL-17A, IL-17B, EL-17C, IL-17D, IL-17E, IL-17F, IL-18, IL-19, IL-20, IL-21, IL-22, IL-23, IL-24, IL-25, IL-26, IL-27, IL-28A/B, IL-29, IL-30, IL-31, IL-32, IL-33, IL-35.

In some aspects, the the cytokine may be one or more cytokines chosen from the TNF family, e.g. chosen from the following list: TNF, especially TNFα, LTα, LTβ, LIGHT, TWEAK, APRIL, BAFF, TL1A, GITRL, OX40L, CD40L (CD154), FASL, CD27L, CD30L, 4-1BBL, TRAIL, RANK ligand. Further examples of preferred cytokines may be chosen from the following list: FLT3 ligand, G-CSF, GM-CSF, IFNα/β/ω, IFNγ, LIF, M-CSF, MIF, OSM, Stem Cell Factor, TGFβ1, TGFβ2, TGFβ3, TSLP ligand.

In some aspects, a cell expressing dnVSIG3 and/or dnVSIG8 exhibits a reduction in the amount of one or more chemokines produced by the cell when the cell is in the presence of a cognate ligand that binds with the extracellular domain of dnVSIG3 and/or dnVSIG8, as compared to the wild-type form of the corresponding VSIG3 or VSIG8.

In some aspects, the one or more chemokines may be chosen from CCL1, CCL11, CCL12, CCL13, CCL14, CCL15, CCL16, CCL17, CCL18, CCL19, CCL2, CCL20, CCL21, CCL22, CCL24, CCL25, CCL26, CCL27, CCL28, CCL3, CCL4, CCL5, CCL6, CCL7, CCL8, CCL9, CX3CL1, CXCL1, CXCL10, CXCL11, CXCL12, CXCL13, CXCL14, CXCL15, CXCL2, CXCL3, CXCL4, CXCL5, CXCL6, CXCL7, CXCL8, CXCL9, XCL1, and XCL2.

In some aspects, the reduction in the amount of one or more cytokines and/or chemokines produced by a cell expressing dnVSIG3 and/or dnVSIG8 when the cell is in the presence of a cognate ligand that binds with the extracellular domain of dnVSIG3 and/or dnVSIG8 is a reduction of at least about

In some aspects, this reduction in the amount of any one or more cytokines and/or any one or more chemokines, relative to an unmodified VSIG3 or VSIG8, is a reduction of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95%.

In some aspects, this reduction in the amount of any one or more cytokines and/or any one or more chemokines, relative to an unmodified VSIG3 or VSIG8, is a reduction of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, or about 95%.

### IV. Vectors and Cells

A nucleic acid of the present disclosure may be present within an expression vector and/or a cloning vector. An expression vector can include a selectable marker, an origin of replication, and other features that provide for replication and/or maintenance of the vector. Suitable expression vectors include, e.g., plasmids, viral vectors, and the like. Large numbers of suitable vectors and promoters are known to those of skill in the art; many are commercially available for generating a subject recombinant construct. The following vectors are provided by way of example, and should not be construed in anyway as limiting: Bacterial: pBs, phagescript, PsiX174, pBluescript SK, pBs KS, pNH8a, pNH16a, pNH18a, pNH46a (Stratagene, La Jolla, Calif., USA); pTrc99A, pKK223-3, pKK233-3, pDR540, and pRIT5 (Pharmacia, Uppsala, Sweden). Eukaryotic: pWLneo, pSV2cat, pOG44, PXR1, pSG (Stratagene) pSVK3, pBPV, pMSG and pSVL (Pharmacia).

Expression vectors generally have convenient restriction sites located near the promoter sequence to provide for the insertion of nucleic acid sequences encoding heterologous proteins. A selectable marker operative in the expression host may be present. Suitable expression vectors include, but are not limited to, viral vectors (e.g. viral vectors based on vaccinia virus; poliovirus; adenovirus (see, e.g., Li et al., Invest. Opthalmol. Vis. Sci. (1994) 35:2543-2549; Borras et al., Gene Ther. (1999) 6:515-524; Li and Davidson, Proc. Natl. Acad. Sci. USA (1995) 92:7700-7704; Sakamoto et al., H. Gene Ther. (1999) 5:1088-1097; WO 94/12649, WO 93/03769; WO 93/19191; WO 94/28938; WO 95/11984 and WO 95/00655); adeno-associated virus (see, e.g., Ali et al., Hum. Gene Ther. (1998) 9:81-86, Flannery et al., Proc. Natl. Acad. Sci. USA (1997) 94:6916-6921; Bennett et al., Invest. Opthalmol. Vis. Sci. (1997) 38:2857-2863; Jomary et al., Gene Ther. (1997) 4:683 690, Rolling et al., Hum. Gene Ther. (1999) 10:641-648; Ali et al., Hum. Mol. Genet. (1996) 5:591-594; Srivastava in WO 93/09239, Samulski et al., J. Vir. (1989) 63:3822-3828; Mendelson et al., Virol. (1988) 166:154-165; and Flotte et al., Proc. Natl. Acad. Sci. USA (1993) 90:10613-10617); SV40; herpes simplex virus; human immunodeficiency virus (see, e.g., Miyoshi et al., Proc. Natl. Acad. Sci. USA (1997) 94:10319-23; Takahashi et al., J. Virol. (1999) 73:7812-7816); a retroviral vector (e.g., Murine Leukemia Virus, spleen necrosis virus, and vectors derived from retroviruses such as Rous Sarcoma Virus, Harvey Sarcoma Virus, avian leukosis virus, human immunodeficiency virus, myeloproliferative sarcoma virus, and mammary tumor virus); and the like.

Additional expression vectors suitable for use are, e.g., without limitation, a lentivirus vector, a gamma retrovirus vector, a foamy virus vector, an adeno-associated virus vector, an adenovirus vector, a pox virus vector, a herpes virus vector, an engineered hybrid virus vector, a transposon mediated vector, and the like. Viral vector technology is well known in the art and is described, for example, in Sambrook et al., 2012, Molecular Cloning: A Laboratory Manual, volumes 1-4, Cold Spring Harbor Press, NY), and in other virology and molecular biology manuals. Viruses, which are useful as vectors include, but are not limited to, retroviruses, adenoviruses, adeno-associated viruses, herpes viruses, and lentiviruses.

In general, a suitable vector contains an origin of replication functional in at least one organism, a promoter sequence, convenient restriction endonuclease sites, and one or more selectable markers, (e.g., WO 01/96584; WO 01/29058; and U.S. Pat. No. 6,326,193).

In some embodiments, an expression vector (e.g., a lentiviral vector) may be used to introduce the the dominant negative receptor into an immune cell or precursor thereof (e.g., a T cell). Accordingly, an expression vector (e.g., a lentiviral vector) of the present invention may comprise a nucleic acid encoding a dominant negative receptor. In some embodiments, the expression vector (e.g., lentiviral vector) will comprise additional elements that will aid in the functional expression of the dominant negative receptor encoded therein. In some embodiments, an expression vector comprising a nucleic acid encoding for the dominant negative receptor further comprises a mammalian promoter. In one embodiment, the vector further comprises an elongation-factor-1-alpha promoter (EF-1α promoter). Use of an EF-1α promoter may increase the efficiency in expression of downstream transgenes (e.g., a dominant negative receptor encoding nucleic acid sequence). Physiologic promoters (e.g., an EF-1α promoter) may be less likely to induce integration mediated genotoxicity, and may abrogate the ability of the retroviral vector to transform stem cells. Other physiological promoters suitable for use in a vector (e.g., lentiviral vector) are known to those of skill in the art and may be incorporated into a vector of the present invention. In some embodiments, the vector (e.g., lentiviral vector) further comprises a non-requisite cis acting sequence that may improve titers and gene expression. One non-limiting example of a non-requisite cis acting sequence is the central polypurine tract and central termination sequence (cPPT/CTS) which is important for efficient reverse transcription and nuclear import. Other non-requisite cis acting sequences are known to those of skill in the art and may be incorporated into a vector (e.g., lentiviral vector) of the present invention. In some embodiments, the vector further comprises a posttranscriptional regulatory element. Posttranscriptional regulatory elements may improve RNA translation, improve transgene expression and stabilize RNA transcripts. One example of a posttranscriptional regulatory element is the woodchuck hepatitis virus posttranscriptional regulatory element (WPRE). Accordingly, in some embodiments a vector for the present invention further comprises a WPRE sequence. Various posttranscriptional regulator elements are known to those of skill in the art and may be incorporated into a vector (e.g., lentiviral vector) of the present invention. A vector of the present invention may further comprise additional elements such as a rev response element (RRE) for RNA transport, packaging sequences, and 5′ and 3′ long terminal repeats (LTRs). The term “long terminal repeat” or “LTR” refers to domains of base pairs located at the ends of retroviral DNAs which comprise U3, R and U5 regions. LTRs generally provide functions required for the expression of retroviral genes (e.g., promotion, initiation and polyadenylation of gene transcripts) and to viral replication. In one embodiment, a vector (e.g., lentiviral vector) of the present invention includes a 3′ U3 deleted LTR. Accordingly, a vector (e.g., lentiviral vector) of the present invention may comprise any combination of the elements described herein to enhance the efficiency of functional expression of transgenes. For example, a vector (e.g., lentiviral vector) of the present invention may comprise a WPRE sequence, cPPT sequence, RRE sequence, 5′LTR, 3′ U3 deleted LTR′ in addition to a nucleic acid encoding for the dominant negative receptor.

Vectors of the present invention may be self-inactivating vectors. As used herein, the term “self-inactivating vector” refers to vectors in which the 3′ LTR enhancer promoter region (U3 region) has been modified (e.g., by deletion or substitution). A self-inactivating vector may prevent viral transcription beyond the first round of viral replication. Consequently, a self-inactivating vector may be capable of infecting and then integrating into a host genome (e.g., a mammalian genome) only once, and cannot be passed further. Accordingly, self-inactivating vectors may greatly reduce the risk of creating a replication-competent virus.

In some embodiments, a nucleic acid of the present invention may be RNA, e.g., in vitro synthesized RNA. Methods for in vitro synthesis of RNA are known to those of skill in the art; any known method can be used to synthesize RNA comprising a sequence encoding a dominant negative receptor of the present disclosure. Methods for introducing RNA into a host cell are known in the art. See, e.g., Zhao et al. Cancer Res. (2010) 15:9053. Introducing RNA comprising a nucleotide sequence encoding a dominant negative receptor of the present disclosure into a host cell can be carried out in vitro or ex vivo or in vivo. For example, a host cell (e.g., an NK cell, a cytotoxic T lymphocyte, etc.) can be electroporated in vitro or ex vivo with RNA comprising a nucleotide sequence encoding a dominant negative receptor of the present disclosure.

To assess the expression of a polypeptide or portions thereof, the expression vector to be introduced into a cell may also contain either a selectable marker gene or a reporter gene, or both, to facilitate identification and selection of expressing cells from the population of cells sought to be transfected or infected through viral vectors. In some embodiments, the selectable marker may be carried on a separate piece of DNA and used in a co-transfection procedure. Both selectable markers and reporter genes may be flanked with appropriate regulatory sequences to enable expression in the host cells. Useful selectable markers include, without limitation, antibiotic-resistance genes.

Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assessed at a suitable time after the DNA has been introduced into the recipient cells. Suitable reporter genes may include, without limitation, genes encoding luciferase, beta-galactosidase, chloramphenicol acetyl transferase, secreted alkaline phosphatase, or the green fluorescent protein gene (e.g., Ui-Tei et al., 2000 FEBS Letters 479:79-82).

In some aspects, the cells comprise dn VSIG3 and/or dnVSIG8. In some aspects, cells comprise polynucleotides encoding dnVSIG3 and/or dnVSIG8. In some aspects, cells comprise vectors that further comprise polynucleotides encoding dnVSIG3 and/or dnVSIG8. In some aspects, cells express dn VSIG3 and/or dnVSIG8.

In some aspects, cells of the present disclosure include higher eukaryotic cells such as a mammalian cell or an insect cell. In some aspects, cells of the present disclosure include lower eukaryotic cells such as a yeast cell. In some aspects, cells of the present disclosure include prokaryotic cells. In some aspects, the cells of the present disclosure include bacterial cells, fungal cells, yeast cells, plant cells, animal cells, and human cells.

In some aspects, the human cells are immune cells. In some aspects, the immune cell is a T cell, e.g., a CD8+ T cell (e.g., a CD8+ naive T cell, central memory T cell, or effector memory T cell), a CD4+ T cell, a natural killer T cell (NKT cells), a regulatory T cell (Treg), a stem cell memory T cell, a lymphoid progenitor cell a hematopoietic stem cell, a natural killer cell (NK cell) or a dendritic cell. In some aspects, the cells are monocytes or granulocytes, e.g., myeloid cells, macrophages, neutrophils, dendritic cells, mast cells, eosinophils, and/or basophils. In some aspects, the cell is an induced pluripotent stem (iPS) cell or a cell derived from an iPS cell, e.g., an iPS cell generated from a subject, manipulated to alter (e.g., induce a mutation in) or manipulate the expression of one or more target genes, and differentiated into, e.g., a T cell, e.g., a CD8+ T cell (e.g., a CD8+ naive T cell, central memory T cell, or effector memory T cell), a CD4+ T cell, a stem cell memory T cell, a lymphoid progenitor cell or a hematopoietic stem cell.

In some aspects, the cells include one or more subsets of T cells or other cell types, such as whole T cell populations, CD4+ cells, CD8+ cells, and subpopulations thereof, such as those defined by function, activation state, maturity, potential for differentiation, expansion, recirculation, localization, and/or persistence capacities, antigen-specificity, type of antigen receptor, presence in a particular organ or compartment, marker or cytokine secretion profile, and/or degree of differentiation. Among the sub-types and subpopulations of T cells and/or of CD4+ and/or of CD8+ T cells are naive T (TN) cells, effector T cells (TEFF), memory T cells and sub-types thereof, such as stem cell memory T (TSCM), central memory T (TCM), effector memory T (TEM), or terminally differentiated effector memory T cells, tumor-infiltrating lymphocytes (TIL), immature T cells, mature T cells, helper T cells, cytotoxic T cells, mucosa-associated invariant T (MATT) cells, naturally occurring and adaptive regulatory T (Treg) cells, helper T cells, such as TH1 cells, TH2 cells, TH3 cells, TH17 cells, TH9 cells, TH22 cells, follicular helper T cells, alpha/beta T cells, and delta/gamma T cells. In some aspects, any number of T cell lines available in the art, may be used.

In some aspects, methods methods of the present disclosure include isolating immune cells from the subject, preparing, processing, culturing, and/or engineering them. In some aspects, preparation of the engineered cells includes one or more culture and/or preparation steps. The cells for engineering as described may be isolated from a sample, such as a biological sample, e.g., one obtained from or derived from a subject. In some aspects, the subject from which the cell is isolated is one having the disease or condition or in need of a cell therapy or to which cell therapy will be administered. The subject in some aspects is a human in need of a particular therapeutic intervention, such as the adoptive cell therapy for which cells are being isolated, processed, and/or engineered. Accordingly, the cells in some aspects are primary cells, e.g., primary human cells. The samples include tissue, fluid, and other samples taken directly from the subject, as well as samples resulting from one or more processing steps, such as separation, centrifugation, genetic engineering (e.g. transduction with viral vector), washing, and/or incubation. The biological sample can be a sample obtained directly from a biological source or a sample that is processed. Biological samples include, but are not limited to, body fluids, such as blood, plasma, serum, cerebrospinal fluid, synovial fluid, urine and sweat, tissue and organ samples, including processed samples derived therefrom.

In some aspects, the sample from which the cells are derived or isolated is blood or a blood-derived sample, or is or is derived from an apheresis or leukapheresis product. Exemplary samples include whole blood, peripheral blood mononuclear cells (PBMCs), leukocytes, bone marrow, thymus, tissue biopsy, tumor, leukemia, lymphoma, lymph node, gut associated lymphoid tissue, mucosa associated lymphoid tissue, spleen, other lymphoid tissues, liver, lung, stomach, intestine, colon, kidney, pancreas, breast, bone, prostate, cervix, testes, ovaries, tonsil, or other organ, and/or cells derived therefrom. Samples include, in the context of cell therapy, e.g., adoptive cell therapy, samples from autologous and allogeneic sources.

### V. Chimeric Antigen Receptors (CARs) and Modified T Cell Receptors

In some aspects, the cells of the present application are modified T cells expressing one or more chimeric antigen receptors (CARs). In some aspects, the cells of the present application are modified T cells expressing one or more modified T cell receptors (TCRs).

In some aspects, the CARs or TCRs comprise binding domains that bind to one or more tumor antigens. In some aspects the tumor antigens are selected from 5T4, 707-AP, 9D7, AFP, AlbZIP HPG1, alpha-5-beta-1-integrin, alpha-5-beta-6-integrin, alpha-actinin-4/m, alpha-methylacyl-coenzyme A racemase, ART-4, ARTC1/m, B7H4, BAGE-1, BCL-2, bcr/abl, beta-catenin/m, BING-4, BRCA1/m, BRCA2/m, CA 15-3/CA 27-29, CA 19-9, CA72-4, CA125, calreticulin, CAMEL, CASP-8/m, cathepsin B, cathepsin L, CD19, CD20, CD22, CD25, CDE30, CD33, CD4, CD52, CD55, CD56, CD80, CDC27/m, CDK4/m, CDKN2A/m, CEA, CLCA2, CML28, CML66, COA-1/m, coactosin-like protein, collage XXIII, COX-2, CT-9/BRD6, Cten, cyclin B1, cyclin D1, cyp-B, CYPB1, DAM-10, DAM-6, DEK-CAN, EFTUD2/m, EGFR, ELF2/m, EMMPRIN, EpCam, EphA2, EphA3, ErbB3, ETV6-AML1, EZH2, FAP, FGF-5, FN, FRα, Frau-1, G250, GAGE-1, GAGE-2, GAGE-3, GAGE-4, GAGE-5, GAGE-6, GAGE7b, GAGE-8, GDEP, GnT-V, gp100, GPC2, GPC3, GPNMB/m, HAGE, HAST-2, hepsin, Her2/neu, HERV-K-MEL, HLA-A*0201-R171, HLA-A11/m, HLA-A2/m, HNE, homeobox NKX3.1, HOM-TES-14/SCP-1, HOM-TES-85, HPV-E6, HPV-E7, HSP70-2M, HST-2, hTERT, iCE, IGF-1R, IL-13Ra2, IL-2R, IL-5, immature laminin receptor, kallikrein-2, kallikrein-4, Ki67, KIAA0205, KIAA0205/m, KK-LC-1, K-Ras/m, LAGE-A1, LDLR-FUT, MAGE-A1, MAGE-A2, MAGE-A3, MAGE-A4, MAGE-A6, MAGE-A9, MAGE-A10, MAGE-A12, MAGE-B1, MAGE-B2, MAGE-B3, MAGE-B4, MAGE-B5, MAGE-B6, MAGE-B10, MAGE-B16, MAGE-B17, MAGE-C1, MAGE-C2, MAGE-C3, MAGE-D1, MAGE-D2, MAGE-D4, MAGE-E1, MAGE-E2, MAGE-F1, MAGE-H1, MAGEL2, mammaglobin A, MART-1/melan-A, MART-2, MART-2/m, matrix protein 22, MC1R, M-CSF, ME1/m, mesothelin, MG50/PXDN, MMP11, MN/CA IX-antigen, MRP-3, MUC-1, Tn Muc-1, MUC-2, MUM-1/m, MUM-2/m, MUM-3/m, myosin class I/m, NA88-A, N-acetylglucosaminyltransferase-V, Neo-PAP, Neo-PAP/m, NFYC/m, NGEP, NMP22, NPM/ALK, N-Ras/m, NSE, NY-ESO—B, NY-ESO-1, OA1, OFA-iLRP, OGT, OGT/m, OS-9, OS-9/m, osteocalcin, osteopontin, p15, p190 minor bcr-abl, p53, p53/m, PAGE-4, PAI-1, PAI-2, PAP, PART-1, PATE, PDEF, Pim-1-Kinase, Pin-1, Pml/PARalpha, POTE, PRAME, PRDX5/m, prostein, proteinase-3, PSA, PSCA, PSGR, PSM, PSMA, PTPRK/m, RAGE-1, RBAF600/m, RHAMM/CD168, RU1, RU2, S-100, SAGE, SART-1, SART-2, SART-3, SCC, SIRT2/m, Sp17, SSX-1, SSX-2/HOM-MEL-40, SSX-4, STAMP-1, STEAP-1, survivin, survivin-2B, SYT-SSX-1, SYT-SSX-2, TA-90, TAG-72, TARP, TEL-AML1, TGFbeta, TGFbetaRll, TGM-4, TPI/m, TRAG-3, TRG, TRP-1, TRP-2/6b, TRP/INT2, TRP-p8, tyrosinase, UPA, VEGFR1, VEGFR-2/FLK-1, and WT1. In some aspects, the CAR comprises one or more binding domains that bind two or at least two of the above antigens. See at least US20190275083A1.

In some aspects, the tumor antigens are preferably selected from the group consisting of p53, CA125, EGFR, Her2/neu, hTERT, PAP, MAGE-A1, MAGE-A3, Mesothelin, MUC-1, GP100, MART-1, Tyrosinase, PSA, PSCA, PSMA, STEAP-1, VEGF, VEGFR1, VEGFR2, Ras, CEA or WT1, and more preferably from PAP, MAGE-A3, WT1, and MUC-1.

In some aspects, the CAR comprises a co-stimulatory domain. In some aspects, the co-stimulatory domain is selected from CD2, 4-1BB, ICOS, and CD27. In some aspects, the CAR comprises two co-stimulatory domains selected from CD2, 4-1BB, ICOS, and CD27. In some aspects, the CAR comprises one or more signalling domains. In some aspects the CAR comprises a CD3ζ signalling domain.

In some aspects, the CAR comprises one or more switch receptors. In some aspects the CAR comprises a second or third dominant-negative receptor that is not a dnVSIG3 or dnVSIG8. In some aspects the receptors are selected from the group consisting of PD1/CD28, PDL1/CD28, CTLA4/CD28, BTLA/CD28, BTLA/ICOS, TIM3/CD28, TIGIT/CD226, dnTGFβ, TGFβ/IL-12R, TGFβ/CD28, TGFβ/OX40, IFNγ/CD28, IFNγ/OX40, and IFNγ/IL-12R.

### VI. Producing Modified Immune Cells

The present disclosure provides methods for producing or generating a modified immune cell or precursor thereof (e.g., a T cell) for tumor immunotherapy, e.g., adoptive immunotherapy. The cells generally are engineered by introducing one or more nucleic acids encoding one or more dominant negative receptors. In some aspects, the one or more nucleic acids encoding one or more dominant negative receptors are introduced into T cells already having been modified to express one or more CARs or one or more TCRs.

In some embodiments, one or more nucleic acids encoding the subject one or more dominant negative receptors are introduced into a cell by an expression vector. Expression vectors comprising a nucleic acid sequence encoding a subject dominant negative receptor of the present invention are provided herein. Suitable expression vectors include lentivirus vectors, gamma retrovirus vectors, foamy virus vectors, adeno associated virus (AAV) vectors, adenovirus vectors, engineered hybrid viruses, naked DNA, including but not limited to transposon mediated vectors, such as Sleeping Beauty, Piggybak, and Integrases such as Phi31. Other suitable expression vectors are described herein.

Expression vectors including a nucleic acid of the present disclosure can be introduced into a host cell by any means known to persons skilled in the art. The expression vectors may include viral sequences for transfection, if desired. Alternatively, the expression vectors may be introduced by fusion, electroporation, biolistics, transfection, lipofection, or the like. The host cell may be grown and expanded in culture before introduction of the expression vectors, followed by the appropriate treatment for introduction and integration of the vectors. The host cells are then expanded and may be screened by virtue of a marker present in the vectors. Various markers that may be used are known in the art, and may include hprt, neomycin resistance, thymidine kinase, hygromycin resistance, etc. As used herein, the terms “cell,” “cell line,” and “cell culture” may be used interchangeably. In some embodiments, the host cell an immune cell or precursor thereof, e.g., a T cell, an NK cell, or an NKT cell.

The present disclosure also provides genetically engineered cells which include one or more stably expressed dominant negative receptors of the present disclosure. In some embodiments, the genetically engineered cells are genetically engineered T-lymphocytes (T cells), naive T cells (TN), memory T cells (for example, central memory T cells (TCM), effector memory cells (TEM)), natural killer cells (NK cells), and macrophages capable of giving rise to therapeutically relevant progeny. In one embodiment, the genetically engineered cells are autologous cells.

Modified cells (e.g., comprising a subject dominant negative receptor) may be produced by stably transfecting host cells with an expression vector including a nucleic acid of the present disclosure. Additional methods to generate a modified cell of the present disclosure include, without limitation, chemical transformation methods (e.g., using calcium phosphate, dendrimers, liposomes and/or cationic polymers), non-chemical transformation methods (e.g., electroporation, optical transformation, gene electrotransfer and/or hydrodynamic delivery) and/or particle-based methods (e.g., impalefection, using a gene gun and/or magnetofection). Transfected cells expressing a subject dominant negative receptor, of the present disclosure may be expanded ex vivo.

Physical methods for introducing an expression vector into host cells include calcium phosphate precipitation, lipofection, particle bombardment, microinjection, electroporation, and the like. Methods for producing cells including vectors and/or exogenous nucleic acids are well-known in the art. See, e.g., Sambrook et al. (2001), Molecular Cloning: A Laboratory Manual, Cold Spring Harbor Laboratory, New York. Chemical methods for introducing an expression vector into a host cell include colloidal dispersion systems, such as macromolecule complexes, nanocapsules, microspheres, beads, and lipid-based systems including oil-in-water emulsions, micelles, mixed micelles, and liposomes.

Lipids suitable for use can be obtained from commercial sources. For example, dimyristyl phosphatidylcholine (“DMPC”) can be obtained from Sigma, St. Louis, Mo.; dicetyl phosphate (“DCP”) can be obtained from K & K Laboratories (Plainview, N.Y.); cholesterol (“Choi”) can be obtained from Calbiochem-Behring; dimyristyl phosphatidylglycerol (“DMPG”) and other lipids may be obtained from Avanti Polar Lipids, Inc. (Birmingham, Ala.). Stock solutions of lipids in chloroform or chloroform/methanol can be stored at about −20° C. Chloroform may be used as the only solvent since it is more readily evaporated than methanol. “Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., 1991 Glycobiology 5:505-10). Compositions that have different structures in solution than the normal vesicular structure are also encompassed. For example, the lipids may assume a micellar structure or merely exist as non-uniform aggregates of lipid molecules. Also contemplated are lipofectamine-nucleic acid complexes.

Regardless of the method used to introduce exogenous nucleic acids into a host cell or otherwise expose a cell to the inhibitor of the present invention, in order to confirm the presence of the nucleic acids in the host cell, a variety of assays may be performed. Such assays include, for example, molecular biology assays well known to those of skill in the art, such as Southern and Northern blotting, RT-PCR and PCR; biochemistry assays, such as detecting the presence or absence of a particular peptide, e.g., by immunological means (ELISAs and Western blots) or by assays described herein to identify agents falling within the scope of the invention.

In some aspects, the nucleic acids introduced into the host cell are RNA. In another embodiment, the RNA is mRNA that comprises in vitro transcribed RNA or synthetic RNA. The RNA may be produced by in vitro transcription using a polymerase chain reaction (PCR)-generated template. DNA of interest from any source can be directly converted by PCR into a template for in vitro mRNA synthesis using appropriate primers and RNA polymerase. The source of the DNA may be, for example, genomic DNA, plasmid DNA, phage DNA, cDNA, synthetic DNA sequence or any other appropriate source of DNA.

PCR may be used to generate a template for in vitro transcription of mRNA which is then introduced into cells. Methods for performing PCR are well known in the art. Primers for use in PCR are designed to have regions that are substantially complementary to regions of the DNA to be used as a template for the PCR. “Substantially complementary,” as used herein, refers to sequences of nucleotides where a majority or all of the bases in the primer sequence are complementary. Substantially complementary sequences are able to anneal or hybridize with the intended DNA target under annealing conditions used for PCR. The primers can be designed to be substantially complementary to any portion of the DNA template. For example, the primers can be designed to amplify the portion of a gene that is normally transcribed in cells (the open reading frame), including 5′ and 3′ UTRs. The primers may also be designed to amplify a portion of a gene that encodes a particular domain of interest. In one embodiment, the primers are designed to amplify the coding region of a human cDNA, including all or portions of the 5′ and 3′ UTRs. Primers useful for PCR are generated by synthetic methods that are well known in the art. “Forward primers” are primers that contain a region of nucleotides that are substantially complementary to nucleotides on the DNA template that are upstream of the DNA sequence that is to be amplified. “Upstream” is used herein to refer to a location 5, to the DNA sequence to be amplified relative to the coding strand. “Reverse primers” are primers that contain a region of nucleotides that are substantially complementary to a double-stranded DNA template that are downstream of the DNA sequence that is to be amplified. “Downstream” is used herein to refer to a location 3′ to the DNA sequence to be amplified relative to the coding strand.

Chemical structures that have the ability to promote stability and/or translation efficiency of the RNA may also be used. The RNA preferably has 5′ and 3′ UTRs. In one embodiment, the 5′ UTR is between zero and 3000 nucleotides in length. The length of 5′ and 3′ UTR sequences to be added to the coding region can be altered by different methods, including, but not limited to, designing primers for PCR that anneal to different regions of the UTRs. Using this approach, one of ordinary skill in the art can modify the 5′ and 3′ UTR lengths required to achieve optimal translation efficiency following transfection of the transcribed RNA.

The 5′ and 3′ UTRs can be the naturally occurring, endogenous 5′ and 3′ UTRs for the gene of interest. Alternatively, UTR sequences that are not endogenous to the gene of interest can be added by incorporating the UTR sequences into the forward and reverse primers or by any other modifications of the template. The use of UTR sequences that are not endogenous to the gene of interest can be useful for modifying the stability and/or translation efficiency of the RNA. For example, it is known that AU-rich elements in 3′ UTR sequences can decrease the stability of mRNA. Therefore, 3′ UTRs can be selected or designed to increase the stability of the transcribed RNA based on properties of UTRs that are well known in the art.

In one embodiment, the 5′ UTR can contain the Kozak sequence of the endogenous gene. Alternatively, when a 5′ UTR that is not endogenous to the gene of interest is being added by PCR as described above, a consensus Kozak sequence can be redesigned by adding the 5′ UTR sequence. Kozak sequences can increase the efficiency of translation of some RNA transcripts, but does not appear to be required for all RNAs to enable efficient translation. The requirement for Kozak sequences for many mRNAs is known in the art. In other embodiments the 5′ UTR can be derived from an RNA virus whose RNA genome is stable in cells. In other embodiments various nucleotide analogues can be used in the 3′ or 5′ UTR to impede exonuclease degradation of the mRNA.

To enable synthesis of RNA from a DNA template without the need for gene cloning, a promoter of transcription should be attached to the DNA template upstream of the sequence to be transcribed. When a sequence that functions as a promoter for an RNA polymerase is added to the 5′ end of the forward primer, the RNA polymerase promoter becomes incorporated into the PCR product upstream of the open reading frame that is to be transcribed. In one embodiment, the promoter is a T7 polymerase promoter, as described elsewhere herein. Other useful promoters include, but are not limited to, T3 and SP6 RNA polymerase promoters. Consensus nucleotide sequences for T7, T3 and SP6 promoters are known in the art.

In one embodiment, the mRNA has both a cap on the 5′ end and a 3′ poly(A) tail which determine ribosome binding, initiation of translation and stability mRNA in the cell. On a circular DNA template, for instance, plasmid DNA, RNA polymerase produces a long concatameric product which is not suitable for expression in eukaryotic cells. The transcription of plasmid DNA linearized at the end of the 3′ UTR results in normal sized mRNA which is not effective in eukaryotic transfection even if it is polyadenylated after transcription.

On a linear DNA template, phage T7 RNA polymerase can extend the 3′ end of the transcript beyond the last base of the template (Schenborn and Mierendorf, Nuc Acids Res., 13:6223-36 (1985); Nacheva and Berzal-Herranz, Eur. J. Biochem., 270:1485-65 (2003).

The polyA/T segment of the transcriptional DNA template can be produced during PCR by using a reverse primer containing a polyT tail, such as 100T tail (size can be 50-5000 T), or after PCR by any other method, including, but not limited to, DNA ligation or in vitro recombination. Poly(A) tails also provide stability to RNAs and reduce their degradation. Generally, the length of a poly(A) tail positively correlates with the stability of the transcribed RNA. In one embodiment, the poly(A) tail is between 100 and 5000 adenosines.

Poly(A) tails of RNAs can be further extended following in vitro transcription with the use of a poly(A) polymerase, such as E. coli polyA polymerase (E-PAP). In one embodiment, increasing the length of a poly(A) tail from 100 nucleotides to between 300 and 400 nucleotides results in about a two-fold increase in the translation efficiency of the RNA. Additionally, the attachment of different chemical groups to the 3′ end can increase mRNA stability. Such attachment can contain modified/artificial nucleotides, aptamers and other compounds. For example, ATP analogs can be incorporated into the poly(A) tail using poly(A) polymerase. ATP analogs can further increase the stability of the RNA.

5′ caps also provide stability to RNA molecules. In a preferred embodiment, RNAs produced by the methods disclosed herein include a 5′ cap. The 5′ cap is provided using techniques known in the art and described herein (Cougot et al., Trends in Biochem. Sci., 29:436-444 (2001); Stepinski, et al., RNA, 7:1468-95 (2001); Elango, et al., Biochim. Biophys. Res. Commun., 330:958-966 (2005)).

In some embodiments, the RNA is electroporated into the cells, such as in vitro transcribed RNA. Any solutes suitable for cell electroporation, which can contain factors facilitating cellular permeability and viability such as sugars, peptides, lipids, proteins, antioxidants, and surfactants can be included.

In some embodiments, a nucleic acid encoding a subject dominant negative receptor, of the present disclosure will be RNA, e.g., in vitro synthesized RNA. Methods for in vitro synthesis of RNA are known in the art; any known method can be used to synthesize RNA comprising a sequence encoding a subject dominant negative receptor. Methods for introducing RNA into a host cell are known in the art. See, e.g., Zhao et al. Cancer Res. (2010) 15:9053. Introducing RNA comprising a nucleotide sequence encoding a dominant negative receptor into a host cell can be carried out in vitro or ex vivo or in vivo. For example, a host cell (e.g., an NK cell, a cytotoxic T lymphocyte, etc.) can be electroporated in vitro or ex vivo with RNA comprising a nucleotide sequence encoding one or more of the subject dominant negative receptors.

The disclosed methods can be applied to the modulation of T cell activity in basic research and therapy, in the fields of cancer, stem cells, acute and chronic infections, and autoimmune diseases, including the assessment of the ability of the genetically modified T cell to kill a target cancer cell.

The methods also provide the ability to control the level of expression over a wide range by changing, for example, the promoter or the amount of input RNA, making it possible to individually regulate the expression level. Furthermore, the PCR-based technique of mRNA production greatly facilitates the design of the mRNAs with different structures and combination of their domains.

One advantage of RNA transfection methods of the invention is that RNA transfection is essentially transient and a vector-free. A RNA transgene can be delivered to a lymphocyte and expressed therein following a brief in vitro cell activation, as a minimal expressing cassette without the need for any additional viral sequences. Under these conditions, integration of the transgene into the host cell genome is unlikely. Cloning of cells is not necessary because of the efficiency of transfection of the RNA and its ability to uniformly modify the entire lymphocyte population.

Genetic modification of T cells with in vitro-transcribed RNA (IVT-RNA) makes use of two different strategies both of which have been successively tested in various animal models. Cells are transfected with in vitro-transcribed RNA by means of lipofection or electroporation. It is desirable to stabilize IVT-RNA using various modifications in order to achieve prolonged expression of transferred IVT-RNA.

Some IVT vectors are known in the literature which are utilized in a standardized manner as template for in vitro transcription and which have been genetically modified in such a way that stabilized RNA transcripts are produced. Currently protocols used in the art are based on a plasmid vector with the following structure: a 5′ RNA polymerase promoter enabling RNA transcription, followed by a gene of interest which is flanked either 3′ and/or 5′ by untranslated regions (UTR), and a 3′ polyadenyl cassette containing 50-70 A nucleotides. Prior to in vitro transcription, the circular plasmid is linearized downstream of the polyadenyl cassette by type II restriction enzymes (recognition sequence corresponds to cleavage site). The polyadenyl cassette thus corresponds to the later poly(A) sequence in the transcript. As a result of this procedure, some nucleotides remain as part of the enzyme cleavage site after linearization and extend or mask the poly(A) sequence at the 3′ end. It is not clear, whether this nonphysiological overhang affects the amount of protein produced intracellularly from such a construct.

In another aspect, the RNA construct is delivered into the cells by electroporation. See, e.g., the formulations and methodology of electroporation of nucleic acid constructs into mammalian cells as taught in US 2004/0014645, US 2005/0052630A1, US 2005/0070841A1, US 2004/0059285A1, US 2004/0092907A1. The various parameters including electric field strength required for electroporation of any known cell type are generally known in the relevant research literature as well as numerous patents and applications in the field. See e.g., U.S. Pat. Nos. 6,678,556, 7,171,264, and 7,173,116. Apparatus for therapeutic application of electroporation are available commercially, e.g., the MedPulser™ DNA Electroporation Therapy System (Inovio/Genetronics, San Diego, Calif.), and are described in patents such as U.S. Pat. Nos. 6,567,694; 6,516,223, 5,993,434, 6,181,964, 6,241,701, and 6,233,482; electroporation may also be used for transfection of cells in vitro as described e.g. in US20070128708A1. Electroporation may also be utilized to deliver nucleic acids into cells in vitro. Accordingly, electroporation-mediated administration into cells of nucleic acids including expression constructs utilizing any of the many available devices and electroporation systems known to those of skill in the art presents an exciting new means for delivering an RNA of interest to a target cell.

In some embodiments, the immune cells (e.g. T cells) can be incubated or cultivated prior to, during and/or subsequent to introducing the nucleic acid molecule encoding the subject dominant negative receptor. In some embodiments, the cells (e.g. T cells) can be incubated or cultivated prior to, during or subsequent to the introduction of the nucleic acid molecule encoding the subject dominant negative receptor, such as prior to, during or subsequent to the transduction of the cells with a viral vector (e.g. lentiviral vector) encoding the subject dominant negative receptor. In some embodiments, the method includes activating or stimulating cells with a stimulating or activating agent (e.g. anti-CD3/anti-CD28 antibodies) prior to introducing the nucleic acid molecule encoding the subject dominant negative receptor.

### VII. Pharmaceutical Compositions

Also provided are populations of immune cells of the present disclosure which comprise one or more dominant-negative receptors, compositions containing such cells and/or enriched for such cells, such as in which cells expressing the recombinant receptor make up at least about 50%, at least about 60%, at least about 70%, at least about 80%, at least about 90%, at least about 91%, at least about 92%, at least about 93%, at least about 94%, at least about 95%, at least about 96%, at least about 97%, at least about 98%, at least about 99%, or more of the total cells in the composition or cells of a certain type such as T cells or CD8+ or CD4+ cells. Among the compositions are pharmaceutical compositions and formulations for administration, such as for adoptive cell therapy. Also provided are therapeutic methods for administering the cells and compositions to subjects, e.g., patients.

Also provided are compositions including the cells for administration, including pharmaceutical compositions and formulations, such as unit dose form compositions including the number of cells for administration in a given dose or fraction thereof. The pharmaceutical compositions and formulations generally include one or more optional pharmaceutically acceptable carrier or excipient. In some embodiments, the composition includes at least one additional therapeutic agent.

The term “pharmaceutical formulation” refers to a preparation which is in such form as to permit the biological activity of an active ingredient contained therein to be effective, and which contains no additional components which are unacceptably toxic to a subject to which the formulation would be administered. A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative. In some aspects, the choice of carrier is determined in part by the particular cell and/or by the method of administration. Accordingly, there are a variety of suitable formulations. For example, the pharmaceutical composition can contain preservatives. Suitable preservatives may include, for example, methylparaben, propylparaben, sodium benzoate, and benzalkonium chloride. In some aspects, a mixture of two or more preservatives is used. The preservative or mixtures thereof are typically present in an amount of about 0.0001% to about 2% by weight of the total composition. Carriers are described, e.g., by Remington's Pharmaceutical Sciences, 16th edition, Osol, A. Ed. (1980). Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG).

Buffering agents in some aspects are included in the compositions. Suitable buffering agents include, for example, citric acid, sodium citrate, phosphoric acid, potassium phosphate, and various other acids and salts. In some aspects, a mixture of two or more buffering agents is used. The buffering agent or mixtures thereof are typically present in an amount of about 0.001% to about 4% by weight of the total composition. Methods for preparing administrable pharmaceutical compositions are known. Exemplary methods are described in more detail in, for example, Remington: The Science and Practice of Pharmacy, Lippincott Williams & Wilkins; 21st ed. (May 1, 2005).

The formulations can include aqueous solutions. The formulation or composition may also contain more than one active ingredient useful for the particular indication, disease, or condition being treated with the cells, preferably those with activities complementary to the cells, where the respective activities do not adversely affect one another. Such active ingredients are suitably present in combination in amounts that are effective for the purpose intended. Thus, in some embodiments, the pharmaceutical composition further includes other pharmaceutically active agents or drugs, such as chemotherapeutic agents, e.g., asparaginase, busulfan, carboplatin, cisplatin, daunorubicin, doxorubicin, fluorouracil, gemcitabine, hydroxyurea, methotrexate, paclitaxel, rituximab, vinblastine, and/or vincristine. The pharmaceutical composition in some embodiments contains the cells in amounts effective to treat or prevent the disease or condition, such as a therapeutically effective or prophylactically effective amount. Therapeutic or prophylactic efficacy in some embodiments is monitored by periodic assessment of treated subjects. The desired dosage can be delivered by a single bolus administration of the cells, by multiple bolus administrations of the cells, or by continuous infusion administration of the cells.

Formulations include those for oral, intravenous, intraperitoneal, subcutaneous, pulmonary, transdermal, intramuscular, intranasal, buccal, sublingual, or suppository administration. In some embodiments, the cell populations are administered parenterally. The term “parenteral,” as used herein, includes intravenous, intramuscular, subcutaneous, rectal, vaginal, and intraperitoneal administration. In some embodiments, the cells are administered to the subject using peripheral systemic delivery by intravenous, intraperitoneal, or subcutaneous injection. Compositions in some embodiments are provided as sterile liquid preparations, e.g., isotonic aqueous solutions, suspensions, emulsions, dispersions, or viscous compositions, which may in some aspects be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol) and suitable mixtures thereof.

Sterile injectable solutions can be prepared by incorporating the cells in a solvent, such as in admixture with a suitable carrier, diluent, or excipient such as sterile water, physiological saline, glucose, dextrose, or the like. The compositions can contain auxiliary substances such as wetting, dispersing, or emulsifying agents (e.g., methylcellulose), pH buffering agents, gelling or viscosity enhancing additives, preservatives, flavoring agents, and/or colors, depending upon the route of administration and the preparation desired. Standard texts may in some aspects be consulted to prepare suitable preparations.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, and sorbic acid. Prolonged absorption of the injectable pharmaceutical form can be brought about by the use of agents delaying absorption, for example, aluminum monostearate and gelatin.

The formulations to be used for in vivo administration are generally sterile. Sterility may be readily accomplished, e.g., by filtration through sterile filtration membranes. In some aspects, formulations to be used for in vivo administration are generally free from microbes. In some aspects, formulations to be used for in vivo administration are generally free from viruses. In some aspects, formulations to be used for in vivo administration are generally free from pathogenic viruses.

### VIII. Methods of Treatment

The modified cells (e.g., T cells) described herein may be included in a composition for immunotherapy. The composition may include a pharmaceutical composition and further include a pharmaceutically acceptable carrier. A therapeutically effective amount of the pharmaceutical composition comprising the modified T cells may be administered.

In one aspect, the invention includes a method for adoptive cell transfer therapy comprising administering to a subject in need thereof a modified T cell of the present invention. In another aspect, the invention includes a method of treating a disease or condition in a subject comprising administering to a subject in need thereof a population of modified T cells.

Also included is a method of treating a disease or condition in a subject in need thereof comprising administering to the subject a modified cell (e.g., modified T cell) of the present invention. In one embodiment, the method of treating a disease or condition in a subject in need thereof comprises administering to the subject a modified cell (e.g., a modified T cell) comprising a subject CAR and/or dominant negative receptor. In one embodiment, the method of treating a disease or condition in a subject in need thereof comprises administering to the subject a modified cell (e.g., a modified T cell) comprising a subject CAR (e.g., a CAR having affinity for PSMA on a target cell) and a dominant negative receptor and/or switch receptor. In one embodiment, the method of treating a disease or condition in a subject in need thereof comprises administering to the subject a modified cell (e.g., a modified T cell) comprising a subject CAR (e.g., a CAR having affinity for PSMA on a target cell), a dominant negative receptor and/or switch receptor, and wherein the modified cell is capable of expressing and secreting a bispecific antibody.

Methods for administration of immune cells for adoptive cell therapy are known and may be used in connection with the provided methods and compositions. For example, adoptive T cell therapy methods are described, e.g., in U.S. Patent Application Publication No. 2003/0170238 to Gruenberg et al; U.S. Pat. No. 4,690,915 to Rosenberg; Rosenberg (2011) Nat Rev Clin Oncol. 8 (10): 577-85). See, e.g., Themeli et al. (2013) Nat Biotechnol. 31 (10): 928-933; Tsukahara et al. (2013) Biochem Biophys Res Commun 438 (1): 84-9; Davila et al. (2013) PLOS ONE 8 (4): e61338. In some embodiments, the cell therapy, e.g., adoptive T cell therapy is carried out by autologous transfer, in which the cells are isolated and/or otherwise prepared from the subject who is to receive the cell therapy, or from a sample derived from such a subject. Thus, in some aspects, the cells are derived from a subject, e.g., patient, in need of a treatment and the cells, following isolation and processing are administered to the same subject.

In some embodiments, the cell therapy, e.g., adoptive T cell therapy, is carried out by allogeneic transfer, in which the cells are isolated and/or otherwise prepared from a subject other than a subject who is to receive or who ultimately receives the cell therapy, e.g., a first subject. In such embodiments, the cells then are administered to a different subject, e.g., a second subject, of the same species. In some embodiments, the first and second subjects are genetically identical. In some embodiments, the first and second subjects are genetically similar. In some embodiments, the second subject expresses the same HLA class or supertype as the first subject.

The modified immune cells of the present invention can be administered to an animal, preferably a mammal, even more preferably a human, to treat a cancer. In addition, the cells of the present invention can be used for the treatment of any condition related to a cancer, especially a cell-mediated immune response against a tumor cell(s), where it is desirable to treat or alleviate the disease. The types of cancers to be treated with the modified cells or pharmaceutical compositions of the invention include, carcinoma, blastoma, and sarcoma, and certain leukemia or lymphoid malignancies, benign and malignant tumors, and malignancies e.g., sarcomas, carcinomas, and melanomas. Other exemplary cancers include but are not limited breast cancer, prostate cancer, ovarian cancer, cervical cancer, skin cancer, pancreatic cancer, colorectal cancer, renal cancer, liver cancer, brain cancer, lymphoma, leukemia, lung cancer, thyroid cancer, and the like. The cancers may be non-solid tumors (such as hematological tumors) or solid tumors. Adult tumors/cancers and pediatric tumors/cancers are also included.

Cells of the invention can be administered in dosages and routes and at times to be determined in appropriate pre-clinical and clinical experimentation and trials. Cell compositions may be administered multiple times at dosages within these ranges. Administration of the cells of the invention may be combined with other methods useful to treat the desired disease or condition as determined by those of skill in the art.

The administration of the cells of the invention may be carried out in any convenient manner known to those of skill in the art. The cells of the present invention may be administered to a subject by aerosol inhalation, injection, ingestion, transfusion, implantation or transplantation. The compositions described herein may be administered to a patient transarterially, subcutaneously, intradermally, intratumorally, intranodally, intramedullary, intramuscularly, by intravenous (i.v.) injection, or intraperitoneally. In other instances, the cells of the invention are injected directly into a site of inflammation in the subject, a local disease site in the subject, a lymph node, an organ, a tumor, and the like.

In certain embodiments, the cells, or individual populations of sub-types of cells, are administered to the subject at a range of about one million to about 100 billion cells, such as, e.g., about 1 million to about 50 billion cells (e.g., about 5 million cells, about 25 million cells, about 500 million cells, about 1 billion cells, about 5 billion cells, about 20 billion cells, about 30 billion cells, about 40 billion cells, or a range defined by any two of the foregoing values), such as about 10 million to about 100 billion cells (e.g., about 20 million cells, about 30 million cells, about 40 million cells, about 60 million cells, about 70 million cells, about 80 million cells, about 90 million cells, about 10 billion cells, about 25 billion cells, about 50 billion cells, about 75 billion cells, about 90 billion cells, or a range defined by any two of the foregoing values), and in some cases about 100 million cells to about 50 billion cells (e.g., about 120 million cells, about 250 million cells, about 350 million cells, about 450 million cells, about 650 million cells, about 800 million cells, about 900 million cells, about 3 billion cells, about 30 billion cells, about 45 billion cells) or any value in between these ranges.

In some embodiments, the dose of total cells and/or dose of individual sub-populations of cells is within a range of between at or about 1×10⁵ cells/kg to about 1×10¹¹ cells/kg, 10⁴, and at or about 10¹¹ cells/kilograms (kg) body weight, such as between about 105 and about 10⁶ cells/kg body weight, for example, at or about 1×10⁵ cells/kg, about 1.5×10⁵ cells/kg, about 2×10⁵ cells/kg, or about 1×10⁶ cells/kg body weight. For example, in some embodiments, the cells are administered at, or within a certain range of error of, between at or about 10⁴ and at or about 10⁹ T cells/kilograms (kg) body weight, such as between about 10⁵ and about 10⁶ T cells/kg body weight, for example, at or about 1×10⁵ T cells/kg, about 1.5×10⁵ T cells/kg, about 2×10⁵ T cells/kg, or about 1×10⁶ T cells/kg body weight. In other exemplary embodiments, a suitable dosage range of modified cells for use in a method of the present disclosure includes, without limitation, from about 1×10⁵ cells/kg to about 1×10⁶ cells/kg, from about 1×10⁶ cells/kg to about 1×10⁷ cells/kg, from about 1×10⁷ cells/kg about 1×10⁸ cells/kg, from about 1×10⁸ cells/kg about 1×10⁹ cells/kg, from about 1×10⁹ cells/kg about 1×10¹⁰ cells/kg, from about 1×10¹⁰ cells/kg about 1×10¹¹ cells/kg. In an exemplary embodiment, a suitable dosage for use in a method of the present disclosure is about 1×10⁸ cells/kg. In an exemplary embodiment, a suitable dosage for use in a method of the present disclosure is about 1×10⁷ cells/kg. In other embodiments, a suitable dosage is from about 1×10⁷ total cells to about 5×10⁷ total cells. In some embodiments, a suitable dosage is from about 1×10⁸ total cells to about 5×10⁸ total cells. In some embodiments, a suitable dosage is from about 1.4×10⁷ total cells to about 1.1×10⁹ total cells. In an exemplary embodiment, a suitable dosage for use in a method of the present disclosure is about 7×10⁹ total cells. In an exemplary embodiment, a suitable dosage is from about 1×10⁷ total cells to about 3×10⁷ total cells.

In some embodiments, a dose of modified cells is administered to a subject in need thereof, in a single dose or multiple doses. In some embodiments, a dose of modified cells is administered in multiple doses, e.g., once a week or about every 7 days, once every 2 weeks or about every 14 days, once every 3 weeks or about every 21 days, once every 4 weeks or about every 28 days. In an exemplary embodiment, a single dose of modified cells is administered to a subject in need thereof. In an exemplary embodiment, a single dose of modified cells is administered to a subject in need thereof by rapid intravenous infusion.

### IX. Definitions

While the following terms are believed to be well understood by one of ordinary skill in the art, the following definitions are set forth to facilitate explanation of the presently disclosed subject matter.

The term “a” or “an” may refer to one or more of that entity, i.e. can refer to plural referents. As such, the terms “a” or “an”, “one or more” and “at least one” are used interchangeably herein. In addition, reference to “an element” by the indefinite article “a” or “an” does not exclude the possibility that more than one of the elements is present, unless the context clearly requires that there is one and only one of the elements.

Reference throughout this specification to “one embodiment”, “an embodiment”, “one aspect”, or “an aspect” means that a particular feature, structure or characteristic described in connection with the embodiment is included in at least one embodiment of the present disclosure. Thus, the appearances of the phrases “in one embodiment” or “in an embodiment” in various places throughout this specification are not necessarily all referring to the same embodiment. Furthermore, the particular features, structures, or characteristics can be combined in any suitable manner in one or more embodiments.

As used herein, the terms “about” or “approximately” when preceding a numerical value indicates the value plus or minus a range of 10% of the value.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

As used herein, a “control” is an alternative sample used in an experiment for comparison purpose. A control can be “positive” or “negative.” A “control sample” or “reference sample” as used herein, refers to a sample or reference that acts as a control for comparison to an experimental sample. For example, an experimental sample comprises compound A, B, and C in a vial, and the control may be the same type of sample treated identically to the experimental sample, but lacking one or more of compounds A, B, or C.

As used herein, the term “effective amount” refers to a quantity sufficient to achieve a desired therapeutic and/or prophylactic effect, e.g., an amount which results in the prevention of one or more outcomes, or an increase in one more outcomes.

As used herein, the terms “individual”, “patient”, or “subject” can be an individual organism, a vertebrate, a mammal, or a human. In a preferred aspect, the individual, patient, or subject is a human.

As used herein, the term “drug,” or “pharmaceutically active agent” or “bioactive agent” or “active agent” as used interchangeably, means any organic or inorganic compound or substance having bioactivity and adapted or used for therapeutic purposes. Proteins, hormones, anti-cancer agents, analgesics, anesthetics, small molecule chemical compounds and mimetics, oligonucleotides, DNA, RNA and gene therapies are included under the broader definition of “drug”. As used herein, reference to a drug, as well as reference to other chemical compounds herein, is meant to include the compound in any of its pharmaceutically acceptable forms, including isomers such as diastereomers and enantiomers, salts, solvates, and polymorphs, particular crystalline forms, as well as racemic mixtures and pure isomers of the compounds described herein, where applicable.

As used herein, the term “injectable” refers to the ability to inject a composition of the present disclosure through a needle.

As used herein, the phrase “dominant-negative receptor” is a variant of a particular receptor comprising dominant-negative mutations that result in modified polypeptides that disrupt the activity of the wild-type receptor. The disruption of the activity may be a complete disruption in the ability of the receptor to participate in signal transduction, or it may be a partial disruption. A dominant-negative receptor further refers to a molecule designed to reduce the effect of a negative signal transduction molecule, e.g. the effect of a negative signal transduction molecule on a modified immune cell of the present disclosure. A modified immune cell comprising a dominant negative receptor may bind a negative signal transduction molecule in the microenvironment of the modified immune cell, and reduce the effect the negative signal transduction molecule may have on the modified immune cell.

As used herein, the phrase “nucleic acid” means any DNA or RNA molecule and is used synonymously with polynucleotide. Wherever herein reference is made to a nucleic acid or nucleic acid sequence encoding a particular protein and/or peptide, said nucleic acid or nucleic acid sequence, respectively, preferably also comprises regulatory sequences allowing in a suitable host, e.g. a human being, its expression, i.e. transcription and/or translation of the nucleic acid sequence encoding the particular protein or peptide.

As used herein the term “peptide” means a polymer of amino acid monomers. Usually the monomers are linked by peptide bonds. The term “peptide” does not limit the length of the polymer chain of amino acids. In some embodiments of the present invention a peptide may for example contain less than 50 monomer units. Longer peptides are also called polypeptides, typically having 50 to 600 monomeric units, more specifically 50 to 300 monomeric units.

As used herein the term “protein” means one or more peptides and/or polypeptides folded into a 3-dimentional form, facilitating a biological function.

As used herein, the phrase “protein variants” or “peptide variants” are proteins/peptides which may be modified relative to the wild-type form and having an amino acid sequence which differs from the original sequence in one or more mutation(s), such as one or more substituted, inserted and/or deleted amino acid(s). In some aspects, these fragments and/or variants have the same biological function or specific activity compared to the full-length native protein, e.g. its specific antigenic property. “Variants” of proteins or peptides as defined in the context of the present invention may comprise conservative amino acid substitution(s) compared to their native, i.e. non-mutated physiological, sequence. Those amino acid sequences as well as their encoding nucleotide sequences in particular fall under the term variants as defined herein. Substitutions in which amino acids, which originate from the same class, are exchanged for one another are called conservative substitutions. In particular, these are amino acids having aliphatic side chains, positively or negatively charged side chains, aromatic groups in the side chains or amino acids, the side chains of which can enter into hydrogen bridges, e.g. side chains which have a hydroxyl function. This means that e.g. an amino acid having a polar side chain is replaced by another amino acid having a likewise polar side chain, or, for example, an amino acid characterized by a hydrophobic side chain is substituted by another amino acid having a likewise hydrophobic side chain (e.g. serine (threonine) by threonine (serine) or leucine (isoleucine) by isoleucine (leucine)). Insertions and substitutions are possible, in particular, at those sequence positions which cause no modification to the three-dimensional structure or do not affect the binding region. Modifications to a three-dimensional structure by insertion(s) or deletion(s) can easily be determined e.g. using CD spectra (circular dichroism spectra).

As used herein, “encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

As used herein, “identity” refers to the subunit sequence identity between two polymeric molecules particularly between two amino acid molecules, such as, between two polypeptide molecules. When two amino acid sequences have the same residues at the same positions; e.g., if a position in each of two polypeptide molecules is occupied by an arginine, then they are identical at that position. The identity or extent to which two amino acid sequences have the same residues at the same positions in an alignment is often expressed as a percentage. The identity between two amino acid sequences is a direct function of the number of matching or identical positions; e.g., if half (e.g., five positions in a polymer ten amino acids in length) of the positions in two sequences are identical, the two sequences are 50% identical; if 90% of the positions (e.g., 9 of 10), are matched or identical, the two amino acids sequences are 90% identical.

As used herein, the phrase “operably linked” or “operatively linked” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence.

As used herein, the term “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, Sendai viral vectors, adenoviral vectors, adeno-associated virus vectors, retroviral vectors, lentiviral vectors, and the like.

“Effective amount” or “therapeutically effective amount” are used interchangeably herein, and refer to an amount of a compound, formulation, material, or composition, as described herein effective to achieve a particular biological result or provides a therapeutic or prophylactic benefit. Such results may include, but are not limited to an amount that when administered to a mammal, causes a detectable level of immune suppression or tolerance compared to the immune response detected in the absence of the composition of the invention. The immune response can be readily assessed by a plethora of art-recognized methods. The skilled artisan would understand that the amount of the composition administered herein varies and can be readily determined based on a number of factors such as the disease or condition being treated, the age and health and physical condition of the mammal being treated, the severity of the disease, the particular compound being administered, and the like.

As used herein, the term “autologous” is meant to refer to any material derived from the same individual to which it is later to be re-introduced into the individual. “Allogeneic” refers to any material derived from a different animal of the same species. “Xenogeneic” refers to any material derived from an animal of a different species.

The term “chimeric antigen receptor” or “CAR,” as used herein refers to an artificial T cell receptor that is engineered to be expressed on an immune cell and specifically bind an antigen. CARs may be used as a therapy with adoptive cell transfer. T cells are removed from a patient and modified so that they express the receptors specific to an antigen or particular form of an antigen. In some embodiments, the CARs have specificity to a selected target, e.g., cells expressing a prostate-specific membrane antigen. CARs may also comprise an intracellular activation domain, a transmembrane domain and an extracellular domain comprising a tumor associated antigen binding region.

The term “downregulation” as used herein refers to the decrease or elimination of gene expression of one or more genes.

The present technology is not to be limited in terms of the particular aspects described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds compositions or biological systems, which can, of course, vary. It is also to be understood that the terminology used herein is for the purpose of describing particular embodiments only, and is not intended to be limiting.

### EXAMPLES

### Example 1

The purpose of this example was to describe preparation of a dn VSIG3.

A dnVSIG3 (SEQ ID NO:20) was created by modifying the amino acid sequence of VSIG3 wild-type variant 6 (SEQ ID NO:6) by truncating the C-terminus of the protein by 166 amino acids. This dnVSIG3 features a near complete loss of the intracellular domain, leaving only 4 amino acids of the intracellular domain.

This example demonstrates the successful preparation of a dnVSIG3.

The methods illustratively described herein may suitably be practiced in the absence of any element or elements, limitation or limitations, not specifically disclosed herein. Thus, for example, the terms “comprising”, “including,” containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof. It is recognized that various modifications are possible within the scope of the disclosure claimed. Thus, it should be understood that although the present disclosure has been specifically disclosed by preferred embodiments and optional features, modification and variation of the disclosure embodied therein herein disclosed may be resorted to by those skilled in the art, and that such modifications and variations are considered to be within the scope of this disclosure.

The disclosure has been described broadly and generically herein. Each of the narrower species and subgeneric groupings falling within the generic disclosure also form part of the methods. This includes the generic description of the methods with a proviso or negative limitation removing any subject matter from the genus, regardless of whether or not the excised material is specifically recited herein. The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds compositions or biological systems, which can, of course, vary. It is also to be understood that the terminology used herein is for the purpose of describing particular embodiments only, and is not intended to be limiting.

One skilled in the art readily appreciates that the present disclosure is well adapted to carry out the objects and obtain the ends and advantages mentioned, as well as those inherent therein. Modifications therein and other uses will occur to those skilled in the art. These modifications are encompassed within the spirit of the disclosure and are defined by the scope of the claims, which set forth non-limiting embodiments of the disclosure.

In addition, where features or aspects of the disclosure are described in terms of Markush groups, those skilled in the art will recognize that the disclosure is also thereby described in terms of any individual member or subgroup of members of the Markush group.

All references, articles, publications, patents, patent publications, and patent applications cited herein are incorporated by reference in their entireties for all purposes.

However, mention of any reference, article, publication, patent, patent publication, and patent application cited herein is not, and should not be taken as an acknowledgment or any form of suggestion that they constitute valid prior art or form part of the common general knowledge in any country in the world.

1. A modified polypeptide comprising an amino acid sequence sharing at least about 90% sequence identity with any one of SEQ ID NOs: 20-30.

2. The polypeptide according to claim 1, wherein:
(a) the amino acid sequence shares at least about 95% sequence identity with any one of SEQ ID NOs: 20-30; and/or
(b) the amino acid sequence shares at least about 95% sequence identity with SEQ ID NO: 20; and/or
(c) the amino acid sequence shares at least about 99% sequence identity with SEQ ID NO: 20; and/or
(d) the amino acid sequence is SEQ ID NO:20.

3. A modified polypeptide comprising an amino acid sequence sharing at least about 90% sequence identity with any one of SEQ ID NOs: 61-70.

4. The polypeptide according to claim 3, wherein:
(a) the amino acid sequence shares at least about 95% sequence identity with any one of SEQ ID NOs: 61-70; and/or
(b) the amino acid sequence shares at least about 95% sequence identity with SEQ ID NO: 61; and/or
(c) the amino acid sequence shares at least about 99% sequence identity with SEQ ID NO: 61; and/or
(d) the amino acid sequence is SEQ ID NO:61.

5. The polypeptide according to claim 1, further comprising a signal peptide sequence.

6. A polynucleotide sequence:
(a) encoding the polypeptide according to claim 1; and/or
(b) comprising a nucleic acid sequence sharing at least about 90% sequence identity with any one of SEQ ID NOs: 50-59; and/or
(c) a polynucleotide sequence comprising a nucleic acid sequence sharing at least about 90% sequence identity with any one of SEQ ID NOs: 72-81.

7. The polynucleotide sequence according to claim 6, wherein:
(a) the nucleic acid sequence of 6 (b) shares at least about 95% sequence identity with any one of SEQ ID NOs: 50-59; and/or
(b) the nucleic acid sequence of 6 (b) shares at least about 99% sequence identity with any one of SEQ ID NOs: 50-59; and/or
(c) the nucleic acid sequence of 6 (b) is SEQ ID NO:50; and/or
(d) the nucleic acid sequence of 6 (c) shares at least about 95% sequence identity with any one of SEQ ID NOs: 72-81; and/or
(e) the nucleic acid sequence of 6 (c) shares at least about 99% sequence identity with any one of SEQ ID NOs: 72-81; and/or
(f) the nucleic acid sequence of 6 (c) is SEQ ID NO:72.

8. A composition comprising
the polypeptide according to claim 1.

9. A vector:
(a) comprising the polynucleotide sequence according to claim 6, and optionally wherein the vector is a viral vector; and/or
(b) comprising the polynucleotide sequence according to claim 6, wherein the polynucleotide sequence is operably linked to one or more polypeptides, and optionally wherein the vector is a viral vector, and/or
(c) comprising the polynucleotide sequence according to claim 6, optionally wherein the polynucleotide sequence is operably linked to one or more polypeptides, wherein the vector is a viral vector, and further wherein the viral vector is selected from the group consisting of lentivirus vector, gamma retrovirus vector, foamy virus vector, adeno-associated virus vector, adenovirus vector, pox virus vector, herpes virus vector, and an engineered hybrid virus vector; and/or
(d) comprising the polynucleotide sequence according to claim 6, optionally wherein the polynucleotide sequence is operably linked to one or more polypeptides, wherein the vector is a lentivirus vector.

10. A cell comprising
the polypeptide according to claim 1, wherein the polypeptide is a dominant-negative receptor as compared to activity of the wild-type VSIG3 receptor.

11. The cell according to claim 10, wherein:
(a) the cell is selected from the group consisting of bacterial cell, fungal cell, yeast cell, animal cell, and human cell; and/or
(b) the cell is a human cell; and/or
(c) the cell is a human cell which is an immune cell; and/or
(d) the cell is a human immune cell, which is a T cell; and/or
(e) the cell is a human immune cell, which is a T cell, wherein the T cell comprises a modified antigen receptor; and/or
(f) the cell is a human immune cell, which is a T cell, wherein the T cell comprises a modified antigen receptor and wherein the modified antigen receptor is a T cell receptor or a chimeric antigen receptor (CAR); and/or
(g) the cell is a human immune cell, which is a T cell, wherein the T cell comprises a modified antigen receptor and wherein the modified antigen receptor is a chimeric antigen receptor (CAR), and the CAR comprises a binder selected from the group consisting of prostate-specific membrane antigen (PSMA), Tn glycoform of mucin 1 (TnMUC1), mesothelin, glypican 2 (GPC2), fibroblast activation protein (FAP), folate receptor alpha (FRα), epidermal growth factor receptor (EGFR), interleukin-13 receptor subunit alpha 2 (IL-13Ralpha2), and any combination thereof; and/or
(h) the cell is a human immune cell, which is a T cell, wherein the T cell comprises a modified antigen receptor and wherein the modified antigen receptor is a chimeric antigen receptor (CAR), and the CAR comprises a binder, wherein the binder comprises a combination of EGFR and IL-13Ralpha2; and/or
(i) the cell is a human immune cell, which is a T cell, wherein the T cell comprises a modified antigen receptor and wherein the modified antigen receptor is a chimeric antigen receptor (CAR), and the CAR comprises a co-stimulatory domain selected from the group consisting of CD2, 4-1BB, ICOS, and CD27; and/or
(j) the cell is a human immune cell, which is a T cell, wherein the T cell comprises a modified antigen receptor and wherein the modified antigen receptor is a chimeric antigen receptor (CAR), and the CAR comprises a switch receptor and/or a dominant negative receptor, wherein the receptors are selected from the group consisting of PD1/CD28, PDL1/CD28, CTLA4/CD28, BTLA/CD28, BTLA/ICOS, TIM3/CD28, TIGIT/CD226, dnTGFβ, TGFβ/IL-12R, TGFβ/CD28, TGFβ/OX40, IFNγ/CD28, IFNγ/OX40, and IFNγ/IL-12R; and/or
(k) the cell is a human immune cell, which is a T cell, wherein the T cell comprises a modified antigen receptor and wherein the modified antigen receptor is a chimeric antigen receptor (CAR), and the CAR comprises a CD35 signaling domain.

12. The cell according to claim 10, wherein:
(a) the dominant-negative receptor is incapable of signal transduction; and/or
(b) the dominant-negative receptor is incapable of signal transduction, and wherein the extracellular domain of the dominant-negative receptor is capable of binding to a corresponding ligand; and/or
(c) the dominant-negative receptor is incapable of signal transduction, and wherein the extracellular domain of the dominant-negative receptor is capable of binding to a corresponding ligand which is VISTA; and/or
(d) expression of the wild-type VSIG3 has been downregulated; and/or
(e) expression of the wild-type VSIG8 has been downregulated.

13. A method of administering the cell according to claim 10, the method comprising administering to a subject a composition comprising the cell, and optionally wherein:
(a) the cell is autologous to the subject; or
(b) the cell is allogeneic to the subject.

14. A method of generating a modified cell, the method comprising introducing into a cell the vector of claim 9.

15. The method according to claim 14, wherein:
(a) the cell is selected from the group consisting of: bacterial cell, fungal cell, yeast cell, animal cell, and human cell; and/or
(b) the cell is a human immune cell; and/or
(c) the cell is a human immune cell, wherein the immune cell is a T cell; and/or
(d) the cell is a human T cell, wherein the T cell comprises a modified antigen receptor; and/or
(e) the cell is a human T cell comprising a modified antigen receptor, wherein the modified antigen receptor is a T cell receptor (TCR) or a chimeric antigen receptor (CAR); and/or
(f) the cell is a human T cell comprising a modified antigen receptor, wherein the modified antigen receptor is a chimeric antigen receptor (CAR), wherein the CAR comprises a binder selected from the group consisting of prostate-specific membrane antigen (PSMA), Tn glycoform of mucin 1 (TnMUC1), mesothelin, glypican 2 (GPC2), fibroblast activation protein (FAP), folate receptor alpha (FRα), and a combination of epidermal growth factor receptor (EGFR) and interleukin-13 receptor subunit alpha 2 (IL-13Ralpha2); and/or
(g) the cell is a human T cell comprising a modified antigen receptor, wherein the modified antigen receptor is a chimeric antigen receptor (CAR), wherein the CAR comprises a binder and wherein the binder comprises a combination of EGFR and IL-13Ralpha2; and/or
(h) the cell is a human T cell comprising a modified antigen receptor, wherein the modified antigen receptor is a chimeric antigen receptor (CAR), wherein the CAR comprises a co-stimulatory domain selected from the group consisting of CD2, 4-1BB, ICOS, and CD27; and/or
(i) the cell is a human T cell comprising a modified antigen receptor, wherein the modified antigen receptor is a chimeric antigen receptor (CAR), wherein the CAR comprises a switch receptor and/or a dominant negative receptor, wherein the receptors are selected from the group consisting of PD1/CD28, PDL1/CD28, CTLA4/CD28, BTLA/CD28, BTLA/ICOS, TIM3/CD28, TIGIT/CD226, dnTGFβ, TGFβ/IL-12R, TGFβ/CD28, TGFβ/OX40, IFNγ/CD28, IFNγ/OX40, and IFNγ/IL-12R; and/or
(j) the cell is a human T cell comprising a modified antigen receptor, wherein the modified antigen receptor is a chimeric antigen receptor (CAR), wherein the CAR comprises a CD35 signaling domain.

16. A composition for use in treating a subject in need, wherein the composition comprises the cell according to claim 10, and optionally wherein:
(a) the cell is autologous to the subject; or
(b) the cell is allogeneic to the subject.

17. The vector according to claim 9, wherein:
(a) the vector is a viral vector;
(b) the vector is a lentivirus vector; and/or
(c) the polynucleotide sequence is operably linked to one or more polypeptides.

18. The vector according to claim 17, wherein the viral vector is selected from the group consisting of lentivirus vector, gamma retrovirus vector, foamy virus vector, adeno-associated virus vector, adenovirus vector, pox virus vector, herpes virus vector, and an engineered hybrid virus vector.

19. The composition for use according to claim 16, wherein:
(a) the cell is autologous to the subject; or
(b) the cell is allogeneic to the subject.

20. The method according to claim 13, wherein:
(a) the cell is autologous to the subject; or
(b) the cell is allogeneic to the subject.


SEQUENCE LISTING (82 sequences)

SEQ ID NO: 1 - protein, 431 aa, Homo sapiens
MTSQRSPLAPLLLLSLHGVAASLEVSESPGSIQVARGQPAVLPCTFTTSAALINLNVIWMVTPLSNANQPEQVILYQGGQMFDGAPRFHGRVGFTGTMPATNVSIFINNTQLSDTGTYQCLVNNLPDIGGRNIGVTGLTVLVPPSAPHCQIQGSQDIGSDVILLCSSEEGIPRPTYLWEKLDNTLKLPPTATQDQVQGTVTIRNISALSSGLYQCVASNAIGTSTCLLDLQVISPQPRNIGLIAGAIGTGAVIIIFCIALILGAFFYWRSKNKEEEEEEIPNEIREDDLPPKCSSAKAFHTEISSSDNNTLTSSNAYNSRYWSNNPKVHRNTESVSHFSDLGQSFSFHSGNANIPSIYANGTHLVPGQHKTLVVTANRGSSPQVMSRSNGSVSRKPRPPHTHSYTISHATLERIGAVPVMVPAQSRAGSLV

SEQ ID NO: 2 - protein, 430 aa, Homo sapiens
MSLVELLLWWNCFSRTGVAASLEVSESPGSIQVARGQPAVLPCTFTTSAALINLNVIWMVTPLSNANQPEQVILYQGGQMFDGAPRFHGRVGFTGTMPATNVSIFINNTQLSDTGTYQCLVNNLPDIGGRNIGVTGLTVLVPPSAPHCQIQGSQDIGSDVILLCSSEEGIPRPTYLWEKLDNTLKLPPTATQDQVQGTVTIRNISALSSGLYQCVASNAIGTSTCLLDLQVISPQPRNIGLIAGAIGTGAVIIIFCIALILGAFFYWRSKNKEEEEEEIPNEIREDDLPPKCSSAKAFHTEISSSDNNTLTSSNAYNSRYWSNNPKVHRNTESVSHFSDLGQSFSFHSGNANIPSIYANGTHLVPGQHKTLVVTANRGSSPQVMSRSNGSVSRKPRPPHTHSYTISHATLERIGAVPVMVPAQSRAGSLV

SEQ ID NO: 3 - protein, 406 aa, Homo sapiens
MSLVELLLWWNCFSRTGVAASLEVSESPGSIQVARGQPAVLPCTFTTSAALINLNVIWMVTPLSNANQPEQVILYQGGQMFDGAPRFHGRVGFTGTMPATNVSIFINNTQLSDTGTYQCLVNNLPDIGGRNIGVTGLTVLVPPSAPHCQIQGSQDIGSDVILLCSSEEGIPRPTYLWEKLDNTLKLPPTATQDQVQGTVTIRNISALSSAQPRNIGLIAGAIGTGAVIIIFCIALILGAFFYWRSKNKEEEEEEIPNEIREDDLPPKCSSAKAFHTEISSSDNNTLTSSNAYNSRYWSNNPKVHRNTESVSHFSDLGQSFSFHSGNANIPSIYANGTHLVPGQHKTLVVTANRGSSPQVMSRSNGSVSRKPRPPHTHSYTISHATLERIGAVPVMVPAQSRAGSLV

SEQ ID NO: 4 - protein, 430 aa, Homo sapiens; identical to SEQ ID NO: 2

SEQ ID NO: 5 - protein, 379 aa, Homo sapiens
MTSQRSPLAPLLLLSLHGVAASLEVSESPGSIQVARGQPAVLPCTFTTSAALINLNVIWMVTPLSNANQPEQVILYQGGQMFDGAPRFHGRVGFTGTMPATNVSIFINNTQLSDTGTYQCLVNNLPDIGGRNIGVTGLTVLVPPSAPHCQIQGSQDIGSDVILLCSSEEGIPRPTYLWEKLDNTLKLPPTATQDQVQGTVTIRNISALSSGAFFYWRSKNKEEEEEEIPNEIREDDLPPKCSSAKAFHTEISSSDNNTLTSSNAYNSRYWSNNPKVHRNTESVSHFSDLGQSFSFHSGNANIPSIYANGTHLVPGQHKTLVVTANRGSSPQVMSRSNGSVSRKPRPPHTHSYTISHATLERIGAVPVMVPAQSRAGSLV

SEQ ID NO: 6 - protein, 407 aa, Homo sapiens
MTSQRSPLAPLLLLSLHGVAASLEVSESPGSIQVARGQPAVLPCTFTTSAALINLNVIWMVTPLSNANQPEQVILYQGGQMFDGAPRFHGRVGFTGTMPATNVSIFINNTQLSDTGTYQCLVNNLPDIGGRNIGVTGLTVLVPPSAPHCQIQGSQDIGSDVILLCSSEEGIPRPTYLWEKLDNTLKLPPTATQDQVQGTVTIRNISALSSAQPRNIGLIAGAIGTGAVIIIFCIALILGAFFYWRSKNKEEEEEEIPNEIREDDLPPKCSSAKAFHTEISSSDNNTLTSSNAYNSRYWSNNPKVHRNTESVSHFSDLGQSFSFHSGNANIPSIYANGTHLVPGQHKTLVVTANRGSSPQVMSRSNGSVSRKPRPPHTHSYTISHATLERIGAVPVMVPAQSRAGSLV

SEQ ID NO: 7 - protein, 403 aa, Homo sapiens
MTSQRSPLAPLLLLSLHGVAASLEVSESPGSIQVARGQPAVLPCTFTTSAALINLNVIWMVTPLSNANQPEQVILYQGGQMFDGAPRFHGRVGFTGTMPATNVSIFINNTQLSDTGTYQCLVNNLPDIGGRNIGVTGLTVLVPPSAPHCQIQGSQDIGSDVILLCSSEEGIPRPTYLWEKLDNTLKLPPTATQDQVQGTVTIRNISALSSGLYQCVASNAIGTSTCLLDLQVISRAFFYWRSKNKEEEEEEIPNEIREDDLPPKCSSAKAFHTEISSSDNNTLTSSNAYNSRYWSNNPKVHRNTESVSHFSDLGQSFSFHSGNANIPSIYANGTHLVPGQHKTLVVTANRGSSPQVMSRSNGSVSRKPRPPHTHSYTISHATLERIGAVPVMVPAQSRAGSLV

SEQ ID NO: 8 - protein, 348 aa, Homo sapiens
MVTPLSNANQPEQVILYQGGQMFDGAPRFHGRVGFTGTMPATNVSIFINNTQLSDTGTYQCLVNNLPDIGGRNIGVTGLTVLVPPSAPHCQIQGSQDIGSDVILLCSSEEGIPRPTYLWEKLDNTLKLPPTATQDQVQGTVTIRNISALSSAQPRNIGLIAGAIGTGAVIIIFCIALILGAFFYWRSKNKEEEEEEIPNEIREDDLPPKCSSAKAFHTEISSSDNNTLTSSNAYNSRYWSNNPKVHRNTESVSHFSDLGQSFSFHSGNANIPSIYANGTHLVPGQHKTLVVTANRGSSPQVMSRSNGSVSRKPRPPHTHSYTISHATLERIGAVPVMVPAQSRAGSLV

SEQ ID NO: 9 - protein, 372 aa, Homo sapiens
MVTPLSNANQPEQVILYQGGQMFDGAPRFHGRVGFTGTMPATNVSIFINNTQLSDTGTYQCLVNNLPDIGGRNIGVTGLTVLVPPSAPHCQIQGSQDIGSDVILLCSSEEGIPRPTYLWEKLDNTLKLPPTATQDQVQGTVTIRNISALSSGLYQCVASNAIGTSTCLLDLQVISPQPRNIGLIAGAIGTGAVIIIFCIALILGAFFYWRSKNKEEEEEEIPNEIREDDLPPKCSSAKAFHTEISSSDNNTLTSSNAYNSRYWSNNPKVHRNTESVSHFSDLGQSFSFHSGNANIPSIYANGTHLVPGQHKTLVVTANRGSSPQVMSRSNGSVSRKPRPPHTHSYTISHATLERIGAVPVMVPAQSRAGSLV

SEQ ID NO: 10 - protein, 453 aa, Homo sapiens
MTSQRSPLAPLLLLSLHDPERRGQHTSGPAGGRGVIQDMYSQLAVGKKTERDRHVGRSLYWGPSCYPSVAASLEVSESPGSIQVARGQPAVLPCTFTTSAALINLNVIWMVTPLSNANQPEQVILYQGGQMFDGAPRFHGRVGFTGTMPATNVSIFINNTQLSDTGTYQCLVNNLPDIGGRNIGVTGLTVLVPPSAPHCQIQGSQDIGSDVILLCSSEEGIPRPTYLWEKLDNTLKLPPTATQDQVQGTVTIRNISALSSGLYQCVASNAIGTSTCLLDLQVISRAFFYWRSKNKEEEEEEIPNEIREDDLPPKCSSAKAFHTEISSSDNNTLTSSNAYNSRYWSNNPKVHRNTESVSHFSDLGQSFSFHSGNANIPSIYANGTHLVPGQHKTLVVTANRGSSPQVMSRSNGSVSRKPRPPHTHSYTISHATLERIGAVPVMVPAQSRAGSLV

SEQ ID NO: 11 - protein, 481 aa, Homo sapiens
MTSQRSPLAPLLLLSLHDPERRGQHTSGPAGGRGVIQDMYSQLAVGKKTERDRHVGRSLYWGPSCYPSVAASLEVSESPGSIQVARGQPAVLPCTFTTSAALINLNVIWMVTPLSNANQPEQVILYQGGQMFDGAPRFHGRVGFTGTMPATNVSIFINNTQLSDTGTYQCLVNNLPDIGGRNIGVTGLTVLVPPSAPHCQIQGSQDIGSDVILLCSSEEGIPRPTYLWEKLDNTLKLPPTATQDQVQGTVTIRNISALSSGLYQCVASNAIGTSTCLLDLQVISPQPRNIGLIAGAIGTGAVIIIFCIALILGAFFYWRSKNKEEEEEEIPNEIREDDLPPKCSSAKAFHTEISSSDNNTLTSSNAYNSRYWSNNPKVHRNTESVSHFSDLGQSFSFHSGNANIPSIYANGTHLVPGQHKTLVVTANRGSSPQVMSRSNGSVSRKPRPPHTHSYTISHATLERIGAVPVMVPAQSRAGSLV

SEQ ID NO: 12 - protein, 480 aa, Homo sapiens
MSLVELLLWWNCFSRTDPERRGQHTSGPAGGRGVIQDMYSQLAVGKKTERDRHVGRSLYWGPSCYPSVAASLEVSESPGSIQVARGQPAVLPCTFTTSAALINLNVIWMVTPLSNANQPEQVILYQGGQMFDGAPRFHGRVGFTGTMPATNVSIFINNTQLSDTGTYQCLVNNLPDIGGRNIGVTGLTVLVPPSAPHCQIQGSQDIGSDVILLCSSEEGIPRPTYLWEKLDNTLKLPPTATQDQVQGTVTIRNISALSSGLYQCVASNAIGTSTCLLDLQVISPQPRNIGLIAGAIGTGAVIIIFCIALILGAFFYWRSKNKEEEEEEIPNEIREDDLPPKCSSAKAFHTEISSSDNNTLTSSNAYNSRYWSNNPKVHRNTESVSHFSDLGQSFSFHSGNANIPSIYANGTHLVPGQHKTLVVTANRGSSPQVMSRSNGSVSRKPRPPHTHSYTISHATLERIGAVPVMVPAQSRAGSLV

SEQ ID NO: 13 - protein, 430 aa, Homo sapiens; identical to SEQ ID NO: 2

SEQ ID NO: 14 - protein, 429 aa, Homo sapiens
MTSQRSPLAPLLLLSLHDPERRGQHTSGPAGGRGVIQDMYSQLAVGKKTERDRHVGRSLYWGPSCYPSVAASLEVSESPGSIQVARGQPAVLPCTFTTSAALINLNVIWMVTPLSNANQPEQVILYQGGQMFDGAPRFHGRVGFTGTMPATNVSIFINNTQLSDTGTYQCLVNNLPDIGGRNIGVTGLTVLVPPSAPHCQIQGSQDIGSDVILLCSSEEGIPRPTYLWEKLDNTLKLPPTATQDQVQGTVTIRNISALSSGAFFYWRSKNKEEEEEEIPNEIREDDLPPKCSSAKAFHTEISSSDNNTLTSSNAYNSRYWSNNPKVHRNTESVSHFSDLGQSFSFHSGNANIPSIYANGTHLVPGQHKTLVVTANRGSSPQVMSRSNGSVSRKPRPPHTHSYTISHATLERIGAVPVMVPAQSRAGSLV

SEQ ID NO: 15 - protein, 457 aa, Homo sapiens
MTSQRSPLAPLLLLSLHDPERRGQHTSGPAGGRGVIQDMYSQLAVGKKTERDRHVGRSLYWGPSCYPSVAASLEVSESPGSIQVARGQPAVLPCTFTTSAALINLNVIWMVTPLSNANQPEQVILYQGGQMFDGAPRFHGRVGFTGTMPATNVSIFINNTQLSDTGTYQCLVNNLPDIGGRNIGVTGLTVLVPPSAPHCQIQGSQDIGSDVILLCSSEEGIPRPTYLWEKLDNTLKLPPTATQDQVQGTVTIRNISALSSAQPRNIGLIAGAIGTGAVIIIFCIALILGAFFYWRSKNKEEEEEEIPNEIREDDLPPKCSSAKAFHTEISSSDNNTLTSSNAYNSRYWSNNPKVHRNTESVSHFSDLGQSFSFHSGNANIPSIYANGTHLVPGQHKTLVVTANRGSSPQVMSRSNGSVSRKPRPPHTHSYTISHATLERIGAVPVMVPAQSRAGSLV

SEQ ID NO: 16 - protein, 443 aa, Homo sapiens
MYSQLAVGKKTERDRHVGRSLYWGPSCYPSVAASLEVSESPGSIQVARGQPAVLPCTFTTSAALINLNVIWMVTPLSNANQPEQVILYQGGQMFDGAPRFHGRVGFTGTMPATNVSIFINNTQLSDTGTYQCLVNNLPDIGGRNIGVTGLTVLVPPSAPHCQIQGSQDIGSDVILLCSSEEGIPRPTYLWEKLDNTLKLPPTATQDQVQGTVTIRNISALSSGLYQCVASNAIGTSTCLLDLQVISPQPRNIGLIAGAIGTGAVIIIFCIALILGAFFYWRSKNKEEEEEEIPNEIREDDLPPKCSSAKAFHTEISSSDNNTLTSSNAYNSRYWSNNPKVHRNTESVSHFSDLGQSFSFHSGNANIPSIYANGTHLVPGQHKTLVVTANRGSSPQVMSRSNGSVSRKPRPPHTHSYTISHATLERIGAVPVMVPAQSRAGSLV

SEQ ID NO: 17 - protein, 372 aa, Homo sapiens; identical to SEQ ID NO: 9

SEQ ID NO: 18 - protein, 323 aa, Homo sapiens
MTSQRSPLAPLLLLSLHDPERRGQHTSGPAGGRGVIQDMYSQLAVGKKTERDRHVGRSLYWGPSCYPSVAASLEVSESPGSIQVARGQPAVLPCTFTTSAALINLNVIWMVTPLSNANQPEQVILYQGGQMFDGAPRFHGRVGFTGTMPATNVSIFINNTQLSDTGTYQCLVNNLPDIGGRNIGVTGLTVLVPPSAPHCQIQGSQDIGSDVILLCSSEEGIPRPTYLWEKLDNTLKLPPTATQDQVQGTVTIRNISALSSGLYQCVASNAIGTSTCLLDLQVISRAFFYWRSKNKEEEEEEIPNEISFIHWIMKEGKVQHVPN

SEQ ID NO: 19 - protein, 351 aa, Homo sapiens
MTSQRSPLAPLLLLSLHDPERRGQHTSGPAGGRGVIQDMYSQLAVGKKTERDRHVGRSLYWGPSCYPSVAASLEVSESPGSIQVARGQPAVLPCTFTTSAALINLNVIWMVTPLSNANQPEQVILYQGGQMFDGAPRFHGRVGFTGTMPATNVSIFINNTQLSDTGTYQCLVNNLPDIGGRNIGVTGLTVLVPPSAPHCQIQGSQDIGSDVILLCSSEEGIPRPTYLWEKLDNTLKLPPTATQDQVQGTVTIRNISALSSGLYQCVASNAIGTSTCLLDLQVISPQPRNIGLIAGAIGTGAVIIIFCIALILGAFFYWRSKNKEEEEEEIPNEISFIHWIMKEGKVQHVPN

SEQ ID NO: 20 - protein, 241 aa, synthetic construct
  REGION 1..241: note = dnVSIG3 mutant of Variant 6
MTSQRSPLAPLLLLSLHGVAASLEVSESPGSIQVARGQPAVLPCTFTTSAALINLNVIWMVTPLSNANQPEQVILYQGGQMFDGAPRFHGRVGFTGTMPATNVSIFINNTQLSDTGTYQCLVNNLPDIGGRNIGVTGLTVLVPPSAPHCQIQGSQDIGSDVILLCSSEEGIPRPTYLWEKLDNTLKLPPTATQDQVQGTVTIRNISALSSAQPRNIGLIAGAIGTGAVIIIFCIALILGAF

SEQ ID NO: 21 - protein, 237 aa, synthetic construct
  source 1..237: note = dnVSIG3 mutant of Variant 6
  REGION 1..237
MTSQRSPLAPLLLLSLHGVAASLEVSESPGSIQVARGQPAVLPCTFTTSAALINLNVIWMVTPLSNANQPEQVILYQGGQMFDGAPRFHGRVGFTGTMPATNVSIFINNTQLSDTGTYQCLVNNLPDIGGRNIGVTGLTVLVPPSAPHCQIQGSQDIGSDVILLCSSEEGIPRPTYLWEKLDNTLKLPPTATQDQVQGTVTIRNISALSSAQPRNIGLIAGAIGTGAVIIIFCIALI

SEQ ID NO: 22 - protein, 239 aa, synthetic construct
  REGION 1..239: note = dnVSIG3 mutant of Variant 6
MTSQRSPLAPLLLLSLHGVAASLEVSESPGSIQVARGQPAVLPCTFTTSAALINLNVIWMVTPLSNANQPEQVILYQGGQMFDGAPRFHGRVGFTGTMPATNVSIFINNTQLSDTGTYQCLVNNLPDIGGRNIGVTGLTVLVPPSAPHCQIQGSQDIGSDVILLCSSEEGIPRPTYLWEKLDNTLKLPPTATQDQVQGTVTIRNISALSSAQPRNIGLIAGAIGTGAVIIIFCIALILG

SEQ ID NO: 23 - protein, 257 aa, synthetic construct
  REGION 1..257: note = dnVSIG3 mutant of Variant 6
MTSQRSPLAPLLLLSLHGVAASLEVSESPGSIQVARGQPAVLPCTFTTSAALINLNVIWMVTPLSNANQPEQVILYQGGQMFDGAPRFHGRVGFTGTMPATNVSIFINNTQLSDTGTYQCLVNNLPDIGGRNIGVTGLTVLVPPSAPHCQIQGSQDIGSDVILLCSSEEGIPRPTYLWEKLDNTLKLPPTATQDQVQGTVTIRNISALSSAQPRNIGLIAGAIGTGAVIIIFCIALILGAFFYWRSKNKEEEEEEIP

SEQ ID NO: 24 - protein, 313 aa, synthetic construct
  REGION 1..313: note = dnVSIG3 mutant of Variant 6
MTSQRSPLAPLLLLSLHGVAASLEVSESPGSIQVARGQPAVLPCTFTTSAALINLNVIWMVTPLSNANQPEQVILYQGGQMFDGAPRFHGRVGFTGTMPATNVSIFINNTQLSDTGTYQCLVNNLPDIGGRNIGVTGLTVLVPPSAPHCQIQGSQDIGSDVILLCSSEEGIPRPTYLWEKLDNTLKLPPTATQDQVQGTVTIRNISALSSAQPRNIGLIAGAIGTGAVIIIFCIALILGAFFYWRSKNKEEEEEEIPNEIREDDLPPKCSSAKAFHTEISSSDNNTLTSSNAYNSRYWSNNPKVHRNTESVSH

SEQ ID NO: 25 - protein, 375 aa, synthetic construct
  REGION 1..375: note = dnVSIG3 mutant of Variant 6
MTSQRSPLAPLLLLSLHGVAASLEVSESPGSIQVARGQPAVLPCTFTTSAALINLNVIWMVTPLSNANQPEQVILYQGGQMFDGAPRFHGRVGFTGTMPATNVSIFINNTQLSDTGTYQCLVNNLPDIGGRNIGVTGLTVLVPPSAPHCQIQGSQDIGSDVILLCSSEEGIPRPTYLWEKLDNTLKLPPTATQDQVQGTVTIRNISALSSAQPRNIGLIAGAIGTGAVIIIFCIALILGAFFYWRSKNKEEEEEEIPNEIREDDLPPKCSSAKAFHTEISSSDNNTLTSSNAYNSRYWSNNPKVHRNTESVSHFSDLGQSFSFHSGNANIPSIYANGTHLVPGQHKTLVVTANRGSSPQVMSRSNGSVSRKPRPP

SEQ ID NO: 26 - protein, 410 aa, synthetic construct
  source 1..410: note = dnVSIG3 mutant of Variant 2
MTSQRSPLAPLLLLSLHGVAASLEVSESPGSIQVARGQPAVLPCTFTTSAALINLNVIWMVTPLSNANQPEQVILYQGGQMFDGAPRFHGRVGFTGTMPATNVSIFINNTQLSDTGTYQCLVNNLPDIGGRNIGVTGLTVLVPPSAPHCQIQGSQDIGSDVILLCSSEEGIPRPTYLWEKLDNTLKLPPTATQDQVQGTVTIRNISALSSGLYQCVASNAIGTSTCLLDLQVISPQPRNIGLIAGAIGTGAVIIIFCIALILGAFFYWRSKNKEEEEEEIPNEIREDDLPPKCSSAKAFHTEISSSDNNTLTSSNAYNSRYWSNNPKVHRNTESVSHFSDLGQSFSFHSGNANIPSIYANGTHLVPGQHKTLVVTANRGSSPQVMSRSNGSVSRKPRPPHTHSYTISHAT

SEQ ID NO: 27 - protein, 357 aa, synthetic construct
  source 1..357: note = dnVSIG3 mutant of Variant 2
MTSQRSPLAPLLLLSLHGVAASLEVSESPGSIQVARGQPAVLPCTFTTSAALINLNVIWMVTPLSNANQPEQVILYQGGQMFDGAPRFHGRVGFTGTMPATNVSIFINNTQLSDTGTYQCLVNNLPDIGGRNIGVTGLTVLVPPSAPHCQIQGSQDIGSDVILLCSSEEGIPRPTYLWEKLDNTLKLPPTATQDQVQGTVTIRNISALSSGLYQCVASNAIGTSTCLLDLQVISPQPRNIGLIAGAIGTGAVIIIFCIALILGAFFYWRSKNKEEEEEEIPNEIREDDLPPKCSSAKAFHTEISSSDNNTLTSSNAYNSRYWSNNPKVHRNTESVSHFSDLGQSFSFHSGNANIPSI

SEQ ID NO: 28 - protein, 338 aa, synthetic construct
  source 1..338: note = dnVSIG3 mutant of Variant 2
MTSQRSPLAPLLLLSLHGVAASLEVSESPGSIQVARGQPAVLPCTFTTSAALINLNVIWMVTPLSNANQPEQVILYQGGQMFDGAPRFHGRVGFTGTMPATNVSIFINNTQLSDTGTYQCLVNNLPDIGGRNIGVTGLTVLVPPSAPHCQIQGSQDIGSDVILLCSSEEGIPRPTYLWEKLDNTLKLPPTATQDQVQGTVTIRNISALSSGLYQCVASNAIGTSTCLLDLQVISPQPRNIGLIAGAIGTGAVIIIFCIALILGAFFYWRSKNKEEEEEEIPNEIREDDLPPKCSSAKAFHTEISSSDNNTLTSSNAYNSRYWSNNPKVHRNTESVSHF

SEQ ID NO: 29 - protein, 272 aa, synthetic construct
  source 1..272: note = dnVSIG3 mutant of Variant 2
MTSQRSPLAPLLLLSLHGVAASLEVSESPGSIQVARGQPAVLPCTFTTSAALINLNVIWMVTPLSNANQPEQVILYQGGQMFDGAPRFHGRVGFTGTMPATNVSIFINNTQLSDTGTYQCLVNNLPDIGGRNIGVTGLTVLVPPSAPHCQIQGSQDIGSDVILLCSSEEGIPRPTYLWEKLDNTLKLPPTATQDQVQGTVTIRNISALSSGLYQCVASNAIGTSTCLLDLQVISPQPRNIGLIAGAIGTGAVIIIFCIALILGAFSRAGSLV

SEQ ID NO: 30 - protein, 352 aa, synthetic construct
  source 1..352: note = dnVSIG3 mutant of Variant 2
MTSQRSPLAPLLLLSLHGVAASLEVSESPGSIQVARGQPAVLPCTFTTSAALINLNVIWMVTPLSNANQPEQVILYQGGQMFDGAPRFHGRVGFTGTMPATNVSIFINNTQLSDTGTYQCLVNNLPDIGGRNIGVTGLTVLVPPSAPHCQIQGSQDIGSDVILLCSSEEGIPRPTYLWEKLDNTLKLPPTATQDQVQGTVTIRNISALSSGLYQCVASNAIGTSTCLLDLQVISPQPRNIGLIAGAIGTGAVIIIFCIALILGAFFYWRSKNKEEEEEEIPNEIREDDLPPKCSSTANRGSSPQVMSRSNGSVSRKPRPPHTHSYTISHATLERIGAVPVMVPAQSRAGSLV

SEQ ID NO: 31 - DNA, 1296 bp, Homo sapiens
  source 1..1296: mol_type = other DNA
atgacttctcagcgttcccctctggcgcctttgctgctcctctctctgcacggtgttgcagcatccctggaagtgtcagagagccctgggagtatccaggtggcccggggtcagccagcagtcctgccctgcactttcactaccagcgctgccctcattaacctcaatgtcatttggatggtcactcctctctccaatgccaaccaacctgaacaggtcatcctgtatcagggtggacagatgtttgatggtgccccccggttccacggtagggtaggatttacaggcaccatgccagctaccaatgtctctatcttcattaataacactcagttatcagacactggcacctaccagtgcctggtcaacaaccttccagacatagggggcaggaacattggggtcaccggtctcacagtgttagttcccccttctgccccacactgccaaatccaaggatcccaggatattggcagcgatgtcatcctgctctgtagctcagaggaaggcattcctcgaccaacttacctttgggagaagttagacaataccctcaaactacctccaacagctactcaggaccaggtccagggaacagtcaccatccggaacatcagtgccctgtcttcaggtttgtaccagtgcgtggcttctaatgctattggaaccagcacctgtcttctggatctccaggttatttcaccccagcccaggaacattggactaatagctggagccattggcactggtgcagttattatcattttttgcattgcactaattttaggggcattcttttactggagaagcaaaaataaagaggaggaagaagaagaaattcctaatgaaataagagaggatgatcttccacccaagtgttcttctgccaaagcatttcacactgagatttcctcctcggacaacaacacactaacctcttccaatgcctacaacagtcgatactggagcaacaatccaaaagttcatagaaacacagagtcagtcagccacttcagtgacttgggccaatctttctctttccactcaggcaatgccaacataccatccatttatgctaatgggacccatctggtcccgggtcaacataagactctggtagtgacagccaacagagggtcatcaccacaggtgatgtccaggagcaatggctcagtcagtaggaagcctcggcctccacacactcattcctacaccatcagccacgcaacactggaacgaattggtgcagtacctgtcatggtaccagcccagagtcgggccgggtccttggtatag

SEQ ID NO: 32 - DNA, 1293 bp, Homo sapiens
  source 1..1293: mol_type = other DNA
atgtctctggtggaacttttgctctggtggaactgcttttctagaactggtgttgcagcatccctggaagtgtcagagagccctgggagtatccaggtggcccggggtcagccagcagtcctgccctgcactttcactaccagcgctgccctcattaacctcaatgtcatttggatggtcactcctctctccaatgccaaccaacctgaacaggtcatcctgtatcagggtggacagatgtttgatggtgccccccggttccacggtagggtaggatttacaggcaccatgccagctaccaatgtctctatcttcattaataacactcagttatcagacactggcacctaccagtgcctggtcaacaaccttccagacatagggggcaggaacattggggtcaccggtctcacagtgttagttcccccttctgccccacactgccaaatccaaggatcccaggatattggcagcgatgtcatcctgctctgtagctcagaggaaggcattcctcgaccaacttacctttgggagaagttagacaataccctcaaactacctccaacagctactcaggaccaggtccagggaacagtcaccatccggaacatcagtgccctgtcttcaggtttgtaccagtgcgtggcttctaatgctattggaaccagcacctgtcttctggatctccaggttatttcaccccagcccaggaacattggactaatagctggagccattggcactggtgcagttattatcattttttgcattgcactaattttaggggcattcttttactggagaagcaaaaataaagaggaggaagaagaagaaattcctaatgaaataagagaggatgatcttccacccaagtgttcttctgccaaagcatttcacactgagatttcctcctcggacaacaacacactaacctcttccaatgcctacaacagtcgatactggagcaacaatccaaaagttcatagaaacacagagtcagtcagccacttcagtgacttgggccaatctttctctttccactcaggcaatgccaacataccatccatttatgctaatgggacccatctggtcccgggtcaacataagactctggtagtgacagccaacagagggtcatcaccacaggtgatgtccaggagcaatggctcagtcagtaggaagcctcggcctccacacactcattcctacaccatcagccacgcaacactggaacgaattggtgcagtacctgtcatggtaccagcccagagtcgggccgggtccttggtatag

SEQ ID NO: 33 - DNA, 1221 bp, Homo sapiens
  source 1..1221: mol_type = other DNA
atgtctctggtggaacttttgctctggtggaactgcttttctagaactggtgttgcagcatccctggaagtgtcagagagccctgggagtatccaggtggcccggggtcagccagcagtcctgccctgcactttcactaccagcgctgccctcattaacctcaatgtcatttggatggtcactcctctctccaatgccaaccaacctgaacaggtcatcctgtatcagggtggacagatgtttgatggtgccccccggttccacggtagggtaggatttacaggcaccatgccagctaccaatgtctctatcttcattaataacactcagttatcagacactggcacctaccagtgcctggtcaacaaccttccagacatagggggcaggaacattggggtcaccggtctcacagtgttagttcccccttctgccccacactgccaaatccaaggatcccaggatattggcagcgatgtcatcctgctctgtagctcagaggaaggcattcctcgaccaacttacctttgggagaagttagacaataccctcaaactacctccaacagctactcaggaccaggtccagggaacagtcaccatccggaacatcagtgccctgtcttcagcccagcccaggaacattggactaatagctggagccattggcactggtgcagttattatcattttttgcattgcactaattttaggggcattcttttactggagaagcaaaaataaagaggaggaagaagaagaaattcctaatgaaataagagaggatgatcttccacccaagtgttcttctgccaaagcatttcacactgagatttcctcctcggacaacaacacactaacctcttccaatgcctacaacagtcgatactggagcaacaatccaaaagttcatagaaacacagagtcagtcagccacttcagtgacttgggccaatctttctctttccactcaggcaatgccaacataccatccatttatgctaatgggacccatctggtcccgggtcaacataagactctggtagtgacagccaacagagggtcatcaccacaggtgatgtccaggagcaatggctcagtcagtaggaagcctcggcctccacacactcattcctacaccatcagccacgcaacactggaacgaattggtgcagtacctgtcatggtaccagcccagagtcgggccgggtccttggtatag

SEQ ID NO: 34 - DNA, 1293 bp, Homo sapiens; identical to SEQ ID NO: 32
  source 1..1293: mol_type = other DNA

SEQ ID NO: 35 - DNA, 1137 bp, Homo sapiens
  source 1..1137: mol_type = other DNA
atgaccagccagaggagccccctggcccccctgctgctgctgagcctgcacggcgtggccgccagcctggaggtgagcgagagccccggcagcatccaggtggccaggggccagcccgccgtgctgccctgcaccttcaccaccagcgccgccctgatcaacctgaacgtgatctggatggtgacccccctgagcaacgccaaccagcccgagcaggtgatcctgtaccagggcggccagatgttcgacggcgcccccaggttccacggcagggtgggcttcaccggcaccatgcccgccaccaacgtgagcatcttcatcaacaacacccagctgagcgacaccggcacctaccagtgcctggtgaacaacctgcccgacatcggcggcaggaacatcggcgtgaccggcctgaccgtgctggtgccccccagcgccccccactgccagatccagggcagccaggacatcggcagcgacgtgatcctgctgtgcagcagcgaggagggcatccccaggcccacctacctgtgggagaagctggacaacaccctgaagctgccccccaccgccacccaggaccaggtgcagggcaccgtgaccatcaggaacatcagcgccctgagcagcggcgccttcttctactggaggagcaagaacaaggaggaggaggaggaggagatccccaacgagatcagggaggacgacctgccccccaagtgcagcagcgccaaggccttccacaccgagatcagcagcagcgacaacaacaccctgaccagcagcaacgcctacaacagcaggtactggagcaacaaccccaaggtgcacaggaacaccgagagcgtgagccacttcagcgacctgggccagagcttcagcttccacagcggcaacgccaacatccccagcatctacgccaacggcacccacctggtgcccggccagcacaagaccctggtggtgaccgccaacaggggcagcagcccccaggtgatgagcaggagcaacggcagcgtgagcaggaagcccaggcccccccacacccacagctacaccatcagccacgccaccctggagaggatcggcgccgtgcccgtgatggtgcccgcccagagcagggccggcagcctggtg

SEQ ID NO: 36 - DNA, 1224 bp, Homo sapiens
  source 1..1224: mol_type = other DNA
atgacttctcagcgttcccctctggcgcctttgctgctcctctctctgcacggtgttgcagcatccctggaagtgtcagagagccctgggagtatccaggtggcccggggtcagccagcagtcctgccctgcactttcactaccagcgctgccctcattaacctcaatgtcatttggatggtcactcctctctccaatgccaaccaacctgaacaggtcatcctgtatcagggtggacagatgtttgatggtgccccccggttccacggtagggtaggatttacaggcaccatgccagctaccaatgtctctatcttcattaataacactcagttatcagacactggcacctaccagtgcctggtcaacaaccttccagacatagggggcaggaacattggggtcaccggtctcacagtgttagttcccccttctgccccacactgccaaatccaaggatcccaggatattggcagcgatgtcatcctgctctgtagctcagaggaaggcattcctcgaccaacttacctttgggagaagttagacaataccctcaaactacctccaacagctactcaggaccaggtccagggaacagtcaccatccggaacatcagtgccctgtcttcagcccagcccaggaacattggactaatagctggagccattggcactggtgcagttattatcattttttgcattgcactaattttaggggcattcttttactggagaagcaaaaataaagaggaggaagaagaagaaattcctaatgaaataagagaggatgatcttccacccaagtgttcttctgccaaagcatttcacactgagatttcctcctcggacaacaacacactaacctcttccaatgcctacaacagtcgatactggagcaacaatccaaaagttcatagaaacacagagtcagtcagccacttcagtgacttgggccaatctttctctttccactcaggcaatgccaacataccatccatttatgctaatgggacccatctggtcccgggtcaacataagactctggtagtgacagccaacagagggtcatcaccacaggtgatgtccaggagcaatggctcagtcagtaggaagcctcggcctccacacactcattcctacaccatcagccacgcaacactggaacgaattggtgcagtacctgtcatggtaccagcccagagtcgggccgggtccttggtatag

SEQ ID NO: 37 - DNA, 1212 bp, Homo sapiens
  source 1..1212: mol_type = other DNA
atgacttctcagcgttcccctctggcgcctttgctgctcctctctctgcacggtgttgcagcatccctggaagtgtcagagagccctgggagtatccaggtggcccggggtcagccagcagtcctgccctgcactttcactaccagcgctgccctcattaacctcaatgtcatttggatggtcactcctctctccaatgccaaccaacctgaacaggtcatcctgtatcagggtggacagatgtttgatggtgccccccggttccacggtagggtaggatttacaggcaccatgccagctaccaatgtctctatcttcattaataacactcagttatcagacactggcacctaccagtgcctggtcaacaaccttccagacatagggggcaggaacattggggtcaccggtctcacagtgttagttcccccttctgccccacactgccaaatccaaggatcccaggatattggcagcgatgtcatcctgctctgtagctcagaggaaggcattcctcgaccaacttacctttgggagaagttagacaataccctcaaactacctccaacagctactcaggaccaggtccagggaacagtcaccatccggaacatcagtgccctgtcttcaggtttgtaccagtgcgtggcttctaatgctattggaaccagcacctgtcttctggatctccaggttatttcacgggcattcttttactggagaagcaaaaataaagaggaggaagaagaagaaattcctaatgaaataagagaggatgatcttccacccaagtgttcttctgccaaagcatttcacactgagatttcctcctcggacaacaacacactaacctcttccaatgcctacaacagtcgatactggagcaacaatccaaaagttcatagaaacacagagtcagtcagccacttcagtgacttgggccaatctttctctttccactcaggcaatgccaacataccatccatttatgctaatgggacccatctggtcccgggtcaacataagactctggtagtgacagccaacagagggtcatcaccacaggtgatgtccaggagcaatggctcagtcagtaggaagcctcggcctccacacactcattcctacaccatcagccacgcaacactggaacgaattggtgcagtacctgtcatggtaccagcccagagtcgggccgggtccttggtatag

SEQ ID NO: 38 - DNA, 1044 bp, Homo sapiens
  source 1..1044: mol_type = other DNA
atggtgacccccctgagcaacgccaaccagcccgagcaggtgatcctgtaccagggcggccagatgttcgacggcgcccccaggttccacggcagggtgggcttcaccggcaccatgcccgccaccaacgtgagcatcttcatcaacaacacccagctgagcgacaccggcacctaccagtgcctggtgaacaacctgcccgacatcggcggcaggaacatcggcgtgaccggcctgaccgtgctggtgccccccagcgccccccactgccagatccagggcagccaggacatcggcagcgacgtgatcctgctgtgcagcagcgaggagggcatccccaggcccacctacctgtgggagaagctggacaacaccctgaagctgccccccaccgccacccaggaccaggtgcagggcaccgtgaccatcaggaacatcagcgccctgagcagcgcccagcccaggaacatcggcctgatcgccggcgccatcggcaccggcgccgtgatcatcatcttctgcatcgccctgatcctgggcgccttcttctactggaggagcaagaacaaggaggaggaggaggaggagatccccaacgagatcagggaggacgacctgccccccaagtgcagcagcgccaaggccttccacaccgagatcagcagcagcgacaacaacaccctgaccagcagcaacgcctacaacagcaggtactggagcaacaaccccaaggtgcacaggaacaccgagagcgtgagccacttcagcgacctgggccagagcttcagcttccacagcggcaacgccaacatccccagcatctacgccaacggcacccacctggtgcccggccagcacaagaccctggtggtgaccgccaacaggggcagcagcccccaggtgatgagcaggagcaacggcagcgtgagcaggaagcccaggcccccccacacccacagctacaccatcagccacgccaccctggagaggatcggcgccgtgcccgtgatggtgcccgcccagagcagggccggcagcctggtg

SEQ ID NO: 39 - DNA, 1116 bp, Homo sapiens
  source 1..1116: mol_type = other DNA
atggtgacccccctgagcaacgccaaccagcccgagcaggtgatcctgtaccagggcggccagatgttcgacggcgcccccaggttccacggcagggtgggcttcaccggcaccatgcccgccaccaacgtgagcatcttcatcaacaacacccagctgagcgacaccggcacctaccagtgcctggtgaacaacctgcccgacatcggcggcaggaacatcggcgtgaccggcctgaccgtgctggtgccccccagcgccccccactgccagatccagggcagccaggacatcggcagcgacgtgatcctgctgtgcagcagcgaggagggcatccccaggcccacctacctgtgggagaagctggacaacaccctgaagctgccccccaccgccacccaggaccaggtgcagggcaccgtgaccatcaggaacatcagcgccctgagcagcggcctgtaccagtgcgtggccagcaacgccatcggcaccagcacctgcctgctggacctgcaggtgatcagcccccagcccaggaacatcggcctgatcgccggcgccatcggcaccggcgccgtgatcatcatcttctgcatcgccctgatcctgggcgccttcttctactggaggagcaagaacaaggaggaggaggaggaggagatccccaacgagatcagggaggacgacctgccccccaagtgcagcagcgccaaggccttccacaccgagatcagcagcagcgacaacaacaccctgaccagcagcaacgcctacaacagcaggtactggagcaacaaccccaaggtgcacaggaacaccgagagcgtgagccacttcagcgacctgggccagagcttcagcttccacagcggcaacgccaacatccccagcatctacgccaacggcacccacctggtgcccggccagcacaagaccctggtggtgaccgccaacaggggcagcagcccccaggtgatgagcaggagcaacggcagcgtgagcaggaagcccaggcccccccacacccacagctacaccatcagccacgccaccctggagaggatcggcgccgtgcccgtgatggtgcccgcccagagcagggccggcagcctggtg

SEQ ID NO: 40 - DNA, 1359 bp, Homo sapiens
  source 1..1359: mol_type = other DNA
atgaccagccagaggagccccctggcccccctgctgctgctgagcctgcacgaccccgagaggaggggccagcacaccagcggccccgccggcggcaggggcgtgatccaggacatgtacagccagctggccgtgggcaagaagaccgagagggacaggcacgtgggcaggagcctgtactggggccccagctgctaccccagcgtggccgccagcctggaggtgagcgagagccccggcagcatccaggtggccaggggccagcccgccgtgctgccctgcaccttcaccaccagcgccgccctgatcaacctgaacgtgatctggatggtgacccccctgagcaacgccaaccagcccgagcaggtgatcctgtaccagggcggccagatgttcgacggcgcccccaggttccacggcagggtgggcttcaccggcaccatgcccgccaccaacgtgagcatcttcatcaacaacacccagctgagcgacaccggcacctaccagtgcctggtgaacaacctgcccgacatcggcggcaggaacatcggcgtgaccggcctgaccgtgctggtgccccccagcgccccccactgccagatccagggcagccaggacatcggcagcgacgtgatcctgctgtgcagcagcgaggagggcatccccaggcccacctacctgtgggagaagctggacaacaccctgaagctgccccccaccgccacccaggaccaggtgcagggcaccgtgaccatcaggaacatcagcgccctgagcagcggcctgtaccagtgcgtggccagcaacgccatcggcaccagcacctgcctgctggacctgcaggtgatcagcagggccttcttctactggaggagcaagaacaaggaggaggaggaggaggagatccccaacgagatcagggaggacgacctgccccccaagtgcagcagcgccaaggccttccacaccgagatcagcagcagcgacaacaacaccctgaccagcagcaacgcctacaacagcaggtactggagcaacaaccccaaggtgcacaggaacaccgagagcgtgagccacttcagcgacctgggccagagcttcagcttccacagcggcaacgccaacatccccagcatctacgccaacggcacccacctggtgcccggccagcacaagaccctggtggtgaccgccaacaggggcagcagcccccaggtgatgagcaggagcaacggcagcgtgagcaggaagcccaggcccccccacacccacagctacaccatcagccacgccaccctggagaggatcggcgccgtgcccgtgatggtgcccgcccagagcagggccggcagcctggtg

SEQ ID NO: 41 - DNA, 1443 bp, Homo sapiens
  source 1..1443: mol_type = other DNA
atgaccagccagaggagccccctggcccccctgctgctgctgagcctgcacgaccccgagaggaggggccagcacaccagcggccccgccggcggcaggggcgtgatccaggacatgtacagccagctggccgtgggcaagaagaccgagagggacaggcacgtgggcaggagcctgtactggggccccagctgctaccccagcgtggccgccagcctggaggtgagcgagagccccggcagcatccaggtggccaggggccagcccgccgtgctgccctgcaccttcaccaccagcgccgccctgatcaacctgaacgtgatctggatggtgacccccctgagcaacgccaaccagcccgagcaggtgatcctgtaccagggcggccagatgttcgacggcgcccccaggttccacggcagggtgggcttcaccggcaccatgcccgccaccaacgtgagcatcttcatcaacaacacccagctgagcgacaccggcacctaccagtgcctggtgaacaacctgcccgacatcggcggcaggaacatcggcgtgaccggcctgaccgtgctggtgccccccagcgccccccactgccagatccagggcagccaggacatcggcagcgacgtgatcctgctgtgcagcagcgaggagggcatccccaggcccacctacctgtgggagaagctggacaacaccctgaagctgccccccaccgccacccaggaccaggtgcagggcaccgtgaccatcaggaacatcagcgccctgagcagcggcctgtaccagtgcgtggccagcaacgccatcggcaccagcacctgcctgctggacctgcaggtgatcagcccccagcccaggaacatcggcctgatcgccggcgccatcggcaccggcgccgtgatcatcatcttctgcatcgccctgatcctgggcgccttcttctactggaggagcaagaacaaggaggaggaggaggaggagatccccaacgagatcagggaggacgacctgccccccaagtgcagcagcgccaaggccttccacaccgagatcagcagcagcgacaacaacaccctgaccagcagcaacgcctacaacagcaggtactggagcaacaaccccaaggtgcacaggaacaccgagagcgtgagccacttcagcgacctgggccagagcttcagcttccacagcggcaacgccaacatccccagcatctacgccaacggcacccacctggtgcccggccagcacaagaccctggtggtgaccgccaacaggggcagcagcccccaggtgatgagcaggagcaacggcagcgtgagcaggaagcccaggcccccccacacccacagctacaccatcagccacgccaccctggagaggatcggcgccgtgcccgtgatggtgcccgcccagagcagggccggcagcctggtg

SEQ ID NO: 42 - DNA, 1440 bp, Homo sapiens
  source 1..1440: mol_type = other DNA
atgagcctggtggagctgctgctgtggtggaactgcttcagcaggaccgaccccgagaggaggggccagcacaccagcggccccgccggcggcaggggcgtgatccaggacatgtacagccagctggccgtgggcaagaagaccgagagggacaggcacgtgggcaggagcctgtactggggccccagctgctaccccagcgtggccgccagcctggaggtgagcgagagccccggcagcatccaggtggccaggggccagcccgccgtgctgccctgcaccttcaccaccagcgccgccctgatcaacctgaacgtgatctggatggtgacccccctgagcaacgccaaccagcccgagcaggtgatcctgtaccagggcggccagatgttcgacggcgcccccaggttccacggcagggtgggcttcaccggcaccatgcccgccaccaacgtgagcatcttcatcaacaacacccagctgagcgacaccggcacctaccagtgcctggtgaacaacctgcccgacatcggcggcaggaacatcggcgtgaccggcctgaccgtgctggtgccccccagcgccccccactgccagatccagggcagccaggacatcggcagcgacgtgatcctgctgtgcagcagcgaggagggcatccccaggcccacctacctgtgggagaagctggacaacaccctgaagctgccccccaccgccacccaggaccaggtgcagggcaccgtgaccatcaggaacatcagcgccctgagcagcggcctgtaccagtgcgtggccagcaacgccatcggcaccagcacctgcctgctggacctgcaggtgatcagcccccagcccaggaacatcggcctgatcgccggcgccatcggcaccggcgccgtgatcatcatcttctgcatcgccctgatcctgggcgccttcttctactggaggagcaagaacaaggaggaggaggaggaggagatccccaacgagatcagggaggacgacctgccccccaagtgcagcagcgccaaggccttccacaccgagatcagcagcagcgacaacaacaccctgaccagcagcaacgcctacaacagcaggtactggagcaacaaccccaaggtgcacaggaacaccgagagcgtgagccacttcagcgacctgggccagagcttcagcttccacagcggcaacgccaacatccccagcatctacgccaacggcacccacctggtgcccggccagcacaagaccctggtggtgaccgccaacaggggcagcagcccccaggtgatgagcaggagcaacggcagcgtgagcaggaagcccaggcccccccacacccacagctacaccatcagccacgccaccctggagaggatcggcgccgtgcccgtgatggtgcccgcccagagcagggccggcagcctggtg

SEQ ID NO: 43 - DNA, 1290 bp, Homo sapiens
  source 1..1290: mol_type = other DNA
atgagcctggtggagctgctgctgtggtggaactgcttcagcaggaccggcgtggccgccagcctggaggtgagcgagagccccggcagcatccaggtggccaggggccagcccgccgtgctgccctgcaccttcaccaccagcgccgccctgatcaacctgaacgtgatctggatggtgacccccctgagcaacgccaaccagcccgagcaggtgatcctgtaccagggcggccagatgttcgacggcgcccccaggttccacggcagggtgggcttcaccggcaccatgcccgccaccaacgtgagcatcttcatcaacaacacccagctgagcgacaccggcacctaccagtgcctggtgaacaacctgcccgacatcggcggcaggaacatcggcgtgaccggcctgaccgtgctggtgccccccagcgccccccactgccagatccagggcagccaggacatcggcagcgacgtgatcctgctgtgcagcagcgaggagggcatccccaggcccacctacctgtgggagaagctggacaacaccctgaagctgccccccaccgccacccaggaccaggtgcagggcaccgtgaccatcaggaacatcagcgccctgagcagcggcctgtaccagtgcgtggccagcaacgccatcggcaccagcacctgcctgctggacctgcaggtgatcagcccccagcccaggaacatcggcctgatcgccggcgccatcggcaccggcgccgtgatcatcatcttctgcatcgccctgatcctgggcgccttcttctactggaggagcaagaacaaggaggaggaggaggaggagatccccaacgagatcagggaggacgacctgccccccaagtgcagcagcgccaaggccttccacaccgagatcagcagcagcgacaacaacaccctgaccagcagcaacgcctacaacagcaggtactggagcaacaaccccaaggtgcacaggaacaccgagagcgtgagccacttcagcgacctgggccagagcttcagcttccacagcggcaacgccaacatccccagcatctacgccaacggcacccacctggtgcccggccagcacaagaccctggtggtgaccgccaacaggggcagcagcccccaggtgatgagcaggagcaacggcagcgtgagcaggaagcccaggcccccccacacccacagctacaccatcagccacgccaccctggagaggatcggcgccgtgcccgtgatggtgcccgcccagagcagggccggcagcctggtg

SEQ ID NO: 44 - DNA, 1287 bp, Homo sapiens
  source 1..1287: mol_type = other DNA
atgaccagccagaggagccccctggcccccctgctgctgctgagcctgcacgaccccgagaggaggggccagcacaccagcggccccgccggcggcaggggcgtgatccaggacatgtacagccagctggccgtgggcaagaagaccgagagggacaggcacgtgggcaggagcctgtactggggccccagctgctaccccagcgtggccgccagcctggaggtgagcgagagccccggcagcatccaggtggccaggggccagcccgccgtgctgccctgcaccttcaccaccagcgccgccctgatcaacctgaacgtgatctggatggtgacccccctgagcaacgccaaccagcccgagcaggtgatcctgtaccagggcggccagatgttcgacggcgcccccaggttccacggcagggtgggcttcaccggcaccatgcccgccaccaacgtgagcatcttcatcaacaacacccagctgagcgacaccggcacctaccagtgcctggtgaacaacctgcccgacatcggcggcaggaacatcggcgtgaccggcctgaccgtgctggtgccccccagcgccccccactgccagatccagggcagccaggacatcggcagcgacgtgatcctgctgtgcagcagcgaggagggcatccccaggcccacctacctgtgggagaagctggacaacaccctgaagctgccccccaccgccacccaggaccaggtgcagggcaccgtgaccatcaggaacatcagcgccctgagcagcggcgccttcttctactggaggagcaagaacaaggaggaggaggaggaggagatccccaacgagatcagggaggacgacctgccccccaagtgcagcagcgccaaggccttccacaccgagatcagcagcagcgacaacaacaccctgaccagcagcaacgcctacaacagcaggtactggagcaacaaccccaaggtgcacaggaacaccgagagcgtgagccacttcagcgacctgggccagagcttcagcttccacagcggcaacgccaacatccccagcatctacgccaacggcacccacctggtgcccggccagcacaagaccctggtggtgaccgccaacaggggcagcagcccccaggtgatgagcaggagcaacggcagcgtgagcaggaagcccaggcccccccacacccacagctacaccatcagccacgccaccctggagaggatcggcgccgtgcccgtgatggtgcccgcccagagcagggccggcagcctggtg

SEQ ID NO: 45 - DNA, 1371 bp, Homo sapiens
  source 1..1371: mol_type = other DNA
atgaccagccagaggagccccctggcccccctgctgctgctgagcctgcacgaccccgagaggaggggccagcacaccagcggccccgccggcggcaggggcgtgatccaggacatgtacagccagctggccgtgggcaagaagaccgagagggacaggcacgtgggcaggagcctgtactggggccccagctgctaccccagcgtggccgccagcctggaggtgagcgagagccccggcagcatccaggtggccaggggccagcccgccgtgctgccctgcaccttcaccaccagcgccgccctgatcaacctgaacgtgatctggatggtgacccccctgagcaacgccaaccagcccgagcaggtgatcctgtaccagggcggccagatgttcgacggcgcccccaggttccacggcagggtgggcttcaccggcaccatgcccgccaccaacgtgagcatcttcatcaacaacacccagctgagcgacaccggcacctaccagtgcctggtgaacaacctgcccgacatcggcggcaggaacatcggcgtgaccggcctgaccgtgctggtgccccccagcgccccccactgccagatccagggcagccaggacatcggcagcgacgtgatcctgctgtgcagcagcgaggagggcatccccaggcccacctacctgtgggagaagctggacaacaccctgaagctgccccccaccgccacccaggaccaggtgcagggcaccgtgaccatcaggaacatcagcgccctgagcagcgcccagcccaggaacatcggcctgatcgccggcgccatcggcaccggcgccgtgatcatcatcttctgcatcgccctgatcctgggcgccttcttctactggaggagcaagaacaaggaggaggaggaggaggagatccccaacgagatcagggaggacgacctgccccccaagtgcagcagcgccaaggccttccacaccgagatcagcagcagcgacaacaacaccctgaccagcagcaacgcctacaacagcaggtactggagcaacaaccccaaggtgcacaggaacaccgagagcgtgagccacttcagcgacctgggccagagcttcagcttccacagcggcaacgccaacatccccagcatctacgccaacggcacccacctggtgcccggccagcacaagaccctggtggtgaccgccaacaggggcagcagcccccaggtgatgagcaggagcaacggcagcgtgagcaggaagcccaggcccccccacacccacagctacaccatcagccacgccaccctggagaggatcggcgccgtgcccgtgatggtgcccgcccagagcagggccggcagcctggtg

SEQ ID NO: 46 - DNA, 1329 bp, Homo sapiens
  source 1..1329: mol_type = other DNA
atgtacagccagctggccgtgggcaagaagaccgagagggacaggcacgtgggcaggagcctgtactggggccccagctgctaccccagcgtggccgccagcctggaggtgagcgagagccccggcagcatccaggtggccaggggccagcccgccgtgctgccctgcaccttcaccaccagcgccgccctgatcaacctgaacgtgatctggatggtgacccccctgagcaacgccaaccagcccgagcaggtgatcctgtaccagggcggccagatgttcgacggcgcccccaggttccacggcagggtgggcttcaccggcaccatgcccgccaccaacgtgagcatcttcatcaacaacacccagctgagcgacaccggcacctaccagtgcctggtgaacaacctgcccgacatcggcggcaggaacatcggcgtgaccggcctgaccgtgctggtgccccccagcgccccccactgccagatccagggcagccaggacatcggcagcgacgtgatcctgctgtgcagcagcgaggagggcatccccaggcccacctacctgtgggagaagctggacaacaccctgaagctgccccccaccgccacccaggaccaggtgcagggcaccgtgaccatcaggaacatcagcgccctgagcagcggcctgtaccagtgcgtggccagcaacgccatcggcaccagcacctgcctgctggacctgcaggtgatcagcccccagcccaggaacatcggcctgatcgccggcgccatcggcaccggcgccgtgatcatcatcttctgcatcgccctgatcctgggcgccttcttctactggaggagcaagaacaaggaggaggaggaggaggagatccccaacgagatcagggaggacgacctgccccccaagtgcagcagcgccaaggccttccacaccgagatcagcagcagcgacaacaacaccctgaccagcagcaacgcctacaacagcaggtactggagcaacaaccccaaggtgcacaggaacaccgagagcgtgagccacttcagcgacctgggccagagcttcagcttccacagcggcaacgccaacatccccagcatctacgccaacggcacccacctggtgcccggccagcacaagaccctggtggtgaccgccaacaggggcagcagcccccaggtgatgagcaggagcaacggcagcgtgagcaggaagcccaggcccccccacacccacagctacaccatcagccacgccaccctggagaggatcggcgccgtgcccgtgatggtgcccgcccagagcagggccggcagcctggtg

SEQ ID NO: 47 - DNA, 1116 bp, Homo sapiens; identical to SEQ ID NO: 39
  source 1..1116: mol_type = other DNA

SEQ ID NO: 48 - DNA, 969 bp, Homo sapiens
  source 1..969: mol_type = other DNA
atgaccagccagaggagccccctggcccccctgctgctgctgagcctgcacgaccccgagaggaggggccagcacaccagcggccccgccggcggcaggggcgtgatccaggacatgtacagccagctggccgtgggcaagaagaccgagagggacaggcacgtgggcaggagcctgtactggggccccagctgctaccccagcgtggccgccagcctggaggtgagcgagagccccggcagcatccaggtggccaggggccagcccgccgtgctgccctgcaccttcaccaccagcgccgccctgatcaacctgaacgtgatctggatggtgacccccctgagcaacgccaaccagcccgagcaggtgatcctgtaccagggcggccagatgttcgacggcgcccccaggttccacggcagggtgggcttcaccggcaccatgcccgccaccaacgtgagcatcttcatcaacaacacccagctgagcgacaccggcacctaccagtgcctggtgaacaacctgcccgacatcggcggcaggaacatcggcgtgaccggcctgaccgtgctggtgccccccagcgccccccactgccagatccagggcagccaggacatcggcagcgacgtgatcctgctgtgcagcagcgaggagggcatccccaggcccacctacctgtgggagaagctggacaacaccctgaagctgccccccaccgccacccaggaccaggtgcagggcaccgtgaccatcaggaacatcagcgccctgagcagcggcctgtaccagtgcgtggccagcaacgccatcggcaccagcacctgcctgctggacctgcaggtgatcagcagggccttcttctactggaggagcaagaacaaggaggaggaggaggaggagatccccaacgagatcagcttcatccactggatcatgaaggagggcaaggtgcagcacgtgcccaac

SEQ ID NO: 49 - DNA, 1053 bp, Homo sapiens
  source 1..1053: mol_type = other DNA
atgaccagccagaggagccccctggcccccctgctgctgctgagcctgcacgaccccgagaggaggggccagcacaccagcggccccgccggcggcaggggcgtgatccaggacatgtacagccagctggccgtgggcaagaagaccgagagggacaggcacgtgggcaggagcctgtactggggccccagctgctaccccagcgtggccgccagcctggaggtgagcgagagccccggcagcatccaggtggccaggggccagcccgccgtgctgccctgcaccttcaccaccagcgccgccctgatcaacctgaacgtgatctggatggtgacccccctgagcaacgccaaccagcccgagcaggtgatcctgtaccagggcggccagatgttcgacggcgcccccaggttccacggcagggtgggcttcaccggcaccatgcccgccaccaacgtgagcatcttcatcaacaacacccagctgagcgacaccggcacctaccagtgcctggtgaacaacctgcccgacatcggcggcaggaacatcggcgtgaccggcctgaccgtgctggtgccccccagcgccccccactgccagatccagggcagccaggacatcggcagcgacgtgatcctgctgtgcagcagcgaggagggcatccccaggcccacctacctgtgggagaagctggacaacaccctgaagctgccccccaccgccacccaggaccaggtgcagggcaccgtgaccatcaggaacatcagcgccctgagcagcggcctgtaccagtgcgtggccagcaacgccatcggcaccagcacctgcctgctggacctgcaggtgatcagcccccagcccaggaacatcggcctgatcgccggcgccatcggcaccggcgccgtgatcatcatcttctgcatcgccctgatcctgggcgccttcttctactggaggagcaagaacaaggaggaggaggaggaggagatccccaacgagatcagcttcatccactggatcatgaaggagggcaaggtgcagcacgtgcccaac

SEQ ID NO: 50 - DNA, 723 bp, synthetic construct
  source 1..723: mol_type = other DNA; note = Mutant of VSIG3 Variant 6
atgaccagccagaggagccccctggcccccctgctgctgctgagcctgcacggcgtggccgccagcctggaggtgagcgagagccccggcagcatccaggtggccaggggccagcccgccgtgctgccctgcaccttcaccaccagcgccgccctgatcaacctgaacgtgatctggatggtgacccccctgagcaacgccaaccagcccgagcaggtgatcctgtaccagggcggccagatgttcgacggcgcccccaggttccacggcagggtgggcttcaccggcaccatgcccgccaccaacgtgagcatcttcatcaacaacacccagctgagcgacaccggcacctaccagtgcctggtgaacaacctgcccgacatcggcggcaggaacatcggcgtgaccggcctgaccgtgctggtgccccccagcgccccccactgccagatccagggcagccaggacatcggcagcgacgtgatcctgctgtgcagcagcgaggagggcatccccaggcccacctacctgtgggagaagctggacaacaccctgaagctgccccccaccgccacccaggaccaggtgcagggcaccgtgaccatcaggaacatcagcgccctgagcagcgcccagcccaggaacatcggcctgatcgccggcgccatcggcaccggcgccgtgatcatcatcttctgcatcgccctgatcctgggcgccttc

SEQ ID NO: 51 - DNA, 711 bp, synthetic construct
  source 1..711: mol_type = other DNA; note = Mutant of VSIG3 Variant 6
atgaccagccagaggagccccctggcccccctgctgctgctgagcctgcacggcgtggccgccagcctggaggtgagcgagagccccggcagcatccaggtggccaggggccagcccgccgtgctgccctgcaccttcaccaccagcgccgccctgatcaacctgaacgtgatctggatggtgacccccctgagcaacgccaaccagcccgagcaggtgatcctgtaccagggcggccagatgttcgacggcgcccccaggttccacggcagggtgggcttcaccggcaccatgcccgccaccaacgtgagcatcttcatcaacaacacccagctgagcgacaccggcacctaccagtgcctggtgaacaacctgcccgacatcggcggcaggaacatcggcgtgaccggcctgaccgtgctggtgccccccagcgccccccactgccagatccagggcagccaggacatcggcagcgacgtgatcctgctgtgcagcagcgaggagggcatccccaggcccacctacctgtgggagaagctggacaacaccctgaagctgccccccaccgccacccaggaccaggtgcagggcaccgtgaccatcaggaacatcagcgccctgagcagcgcccagcccaggaacatcggcctgatcgccggcgccatcggcaccggcgccgtgatcatcatcttctgcatcgccctgatc

SEQ ID NO: 52 - DNA, 717 bp, synthetic construct
  source 1..717: mol_type = other DNA; note = Mutant of VSIG3 Variant 6
atgaccagccagaggagccccctggcccccctgctgctgctgagcctgcacggcgtggccgccagcctggaggtgagcgagagccccggcagcatccaggtggccaggggccagcccgccgtgctgccctgcaccttcaccaccagcgccgccctgatcaacctgaacgtgatctggatggtgacccccctgagcaacgccaaccagcccgagcaggtgatcctgtaccagggcggccagatgttcgacggcgcccccaggttccacggcagggtgggcttcaccggcaccatgcccgccaccaacgtgagcatcttcatcaacaacacccagctgagcgacaccggcacctaccagtgcctggtgaacaacctgcccgacatcggcggcaggaacatcggcgtgaccggcctgaccgtgctggtgccccccagcgccccccactgccagatccagggcagccaggacatcggcagcgacgtgatcctgctgtgcagcagcgaggagggcatccccaggcccacctacctgtgggagaagctggacaacaccctgaagctgccccccaccgccacccaggaccaggtgcagggcaccgtgaccatcaggaacatcagcgccctgagcagcgcccagcccaggaacatcggcctgatcgccggcgccatcggcaccggcgccgtgatcatcatcttctgcatcgccctgatcctgggc

SEQ ID NO: 53 - DNA, 771 bp, synthetic construct
  source 1..771: mol_type = other DNA; note = Mutant of VSIG3 Variant 6
atgaccagccagaggagccccctggcccccctgctgctgctgagcctgcacggcgtggccgccagcctggaggtgagcgagagccccggcagcatccaggtggccaggggccagcccgccgtgctgccctgcaccttcaccaccagcgccgccctgatcaacctgaacgtgatctggatggtgacccccctgagcaacgccaaccagcccgagcaggtgatcctgtaccagggcggccagatgttcgacggcgcccccaggttccacggcagggtgggcttcaccggcaccatgcccgccaccaacgtgagcatcttcatcaacaacacccagctgagcgacaccggcacctaccagtgcctggtgaacaacctgcccgacatcggcggcaggaacatcggcgtgaccggcctgaccgtgctggtgccccccagcgccccccactgccagatccagggcagccaggacatcggcagcgacgtgatcctgctgtgcagcagcgaggagggcatccccaggcccacctacctgtgggagaagctggacaacaccctgaagctgccccccaccgccacccaggaccaggtgcagggcaccgtgaccatcaggaacatcagcgccctgagcagcgcccagcccaggaacatcggcctgatcgccggcgccatcggcaccggcgccgtgatcatcatcttctgcatcgccctgatcctgggcgccttcttctactggaggagcaagaacaaggaggaggaggaggaggagatcccc

SEQ ID NO: 54 - DNA, 1125 bp, synthetic construct
  source 1..1125: mol_type = other DNA; note = Mutant of VSIG3 Variant 6
atgaccagccagaggagccccctggcccccctgctgctgctgagcctgcacggcgtggccgccagcctggaggtgagcgagagccccggcagcatccaggtggccaggggccagcccgccgtgctgccctgcaccttcaccaccagcgccgccctgatcaacctgaacgtgatctggatggtgacccccctgagcaacgccaaccagcccgagcaggtgatcctgtaccagggcggccagatgttcgacggcgcccccaggttccacggcagggtgggcttcaccggcaccatgcccgccaccaacgtgagcatcttcatcaacaacacccagctgagcgacaccggcacctaccagtgcctggtgaacaacctgcccgacatcggcggcaggaacatcggcgtgaccggcctgaccgtgctggtgccccccagcgccccccactgccagatccagggcagccaggacatcggcagcgacgtgatcctgctgtgcagcagcgaggagggcatccccaggcccacctacctgtgggagaagctggacaacaccctgaagctgccccccaccgccacccaggaccaggtgcagggcaccgtgaccatcaggaacatcagcgccctgagcagcgcccagcccaggaacatcggcctgatcgccggcgccatcggcaccggcgccgtgatcatcatcttctgcatcgccctgatcctgggcgccttcttctactggaggagcaagaacaaggaggaggaggaggaggagatccccaacgagatcagggaggacgacctgccccccaagtgcagcagcgccaaggccttccacaccgagatcagcagcagcgacaacaacaccctgaccagcagcaacgcctacaacagcaggtactggagcaacaaccccaaggtgcacaggaacaccgagagcgtgagccacttcagcgacctgggccagagcttcagcttccacagcggcaacgccaacatccccagcatctacgccaacggcacccacctggtgcccggccagcacaagaccctggtggtgaccgccaacaggggcagcagcccccaggtgatgagcaggagcaacggcagcgtgagcaggaagcccaggcccccc

SEQ ID NO: 55 - DNA, 1230 bp, synthetic construct
  source 1..1230: mol_type = other DNA; note = Mutant of VSIG3 Variant 2
atgaccagccagaggagccccctggcccccctgctgctgctgagcctgcacggcgtggccgccagcctggaggtgagcgagagccccggcagcatccaggtggccaggggccagcccgccgtgctgccctgcaccttcaccaccagcgccgccctgatcaacctgaacgtgatctggatggtgacccccctgagcaacgccaaccagcccgagcaggtgatcctgtaccagggcggccagatgttcgacggcgcccccaggttccacggcagggtgggcttcaccggcaccatgcccgccaccaacgtgagcatcttcatcaacaacacccagctgagcgacaccggcacctaccagtgcctggtgaacaacctgcccgacatcggcggcaggaacatcggcgtgaccggcctgaccgtgctggtgccccccagcgccccccactgccagatccagggcagccaggacatcggcagcgacgtgatcctgctgtgcagcagcgaggagggcatccccaggcccacctacctgtgggagaagctggacaacaccctgaagctgccccccaccgccacccaggaccaggtgcagggcaccgtgaccatcaggaacatcagcgccctgagcagcggcctgtaccagtgcgtggccagcaacgccatcggcaccagcacctgcctgctggacctgcaggtgatcagcccccagcccaggaacatcggcctgatcgccggcgccatcggcaccggcgccgtgatcatcatcttctgcatcgccctgatcctgggcgccttcttctactggaggagcaagaacaaggaggaggaggaggaggagatccccaacgagatcagggaggacgacctgccccccaagtgcagcagcgccaaggccttccacaccgagatcagcagcagcgacaacaacaccctgaccagcagcaacgcctacaacagcaggtactggagcaacaaccccaaggtgcacaggaacaccgagagcgtgagccacttcagcgacctgggccagagcttcagcttccacagcggcaacgccaacatccccagcatctacgccaacggcacccacctggtgcccggccagcacaagaccctggtggtgaccgccaacaggggcagcagcccccaggtgatgagcaggagcaacggcagcgtgagcaggaagcccaggcccccccacacccacagctacaccatcagccacgccacc

SEQ ID NO: 56 - DNA, 1071 bp, synthetic construct
  source 1..1071: mol_type = other DNA; note = Mutant of VSIG3 Variant 2
atgaccagccagaggagccccctggcccccctgctgctgctgagcctgcacggcgtggccgccagcctggaggtgagcgagagccccggcagcatccaggtggccaggggccagcccgccgtgctgccctgcaccttcaccaccagcgccgccctgatcaacctgaacgtgatctggatggtgacccccctgagcaacgccaaccagcccgagcaggtgatcctgtaccagggcggccagatgttcgacggcgcccccaggttccacggcagggtgggcttcaccggcaccatgcccgccaccaacgtgagcatcttcatcaacaacacccagctgagcgacaccggcacctaccagtgcctggtgaacaacctgcccgacatcggcggcaggaacatcggcgtgaccggcctgaccgtgctggtgccccccagcgccccccactgccagatccagggcagccaggacatcggcagcgacgtgatcctgctgtgcagcagcgaggagggcatccccaggcccacctacctgtgggagaagctggacaacaccctgaagctgccccccaccgccacccaggaccaggtgcagggcaccgtgaccatcaggaacatcagcgccctgagcagcggcctgtaccagtgcgtggccagcaacgccatcggcaccagcacctgcctgctggacctgcaggtgatcagcccccagcccaggaacatcggcctgatcgccggcgccatcggcaccggcgccgtgatcatcatcttctgcatcgccctgatcctgggcgccttcttctactggaggagcaagaacaaggaggaggaggaggaggagatccccaacgagatcagggaggacgacctgccccccaagtgcagcagcgccaaggccttccacaccgagatcagcagcagcgacaacaacaccctgaccagcagcaacgcctacaacagcaggtactggagcaacaaccccaaggtgcacaggaacaccgagagcgtgagccacttcagcgacctgggccagagcttcagcttccacagcggcaacgccaacatccccagcatc

SEQ ID NO: 57 - DNA, 1014 bp, synthetic construct
  source 1..1014: mol_type = other DNA; note = Mutant of VSIG3 Variant 2
atgaccagccagaggagccccctggcccccctgctgctgctgagcctgcacggcgtggccgccagcctggaggtgagcgagagccccggcagcatccaggtggccaggggccagcccgccgtgctgccctgcaccttcaccaccagcgccgccctgatcaacctgaacgtgatctggatggtgacccccctgagcaacgccaaccagcccgagcaggtgatcctgtaccagggcggccagatgttcgacggcgcccccaggttccacggcagggtgggcttcaccggcaccatgcccgccaccaacgtgagcatcttcatcaacaacacccagctgagcgacaccggcacctaccagtgcctggtgaacaacctgcccgacatcggcggcaggaacatcggcgtgaccggcctgaccgtgctggtgccccccagcgccccccactgccagatccagggcagccaggacatcggcagcgacgtgatcctgctgtgcagcagcgaggagggcatccccaggcccacctacctgtgggagaagctggacaacaccctgaagctgccccccaccgccacccaggaccaggtgcagggcaccgtgaccatcaggaacatcagcgccctgagcagcggcctgtaccagtgcgtggccagcaacgccatcggcaccagcacctgcctgctggacctgcaggtgatcagcccccagcccaggaacatcggcctgatcgccggcgccatcggcaccggcgccgtgatcatcatcttctgcatcgccctgatcctgggcgccttcttctactggaggagcaagaacaaggaggaggaggaggaggagatccccaacgagatcagggaggacgacctgccccccaagtgcagcagcgccaaggccttccacaccgagatcagcagcagcgacaacaacaccctgaccagcagcaacgcctacaacagcaggtactggagcaacaaccccaaggtgcacaggaacaccgagagcgtgagccacttc

SEQ ID NO: 58 - DNA, 816 bp, synthetic construct
  source 1..816: mol_type = other DNA; note = Mutant of VSIG3 Variant 2
atgaccagccagaggagccccctggcccccctgctgctgctgagcctgcacggcgtggccgccagcctggaggtgagcgagagccccggcagcatccaggtggccaggggccagcccgccgtgctgccctgcaccttcaccaccagcgccgccctgatcaacctgaacgtgatctggatggtgacccccctgagcaacgccaaccagcccgagcaggtgatcctgtaccagggcggccagatgttcgacggcgcccccaggttccacggcagggtgggcttcaccggcaccatgcccgccaccaacgtgagcatcttcatcaacaacacccagctgagcgacaccggcacctaccagtgcctggtgaacaacctgcccgacatcggcggcaggaacatcggcgtgaccggcctgaccgtgctggtgccccccagcgccccccactgccagatccagggcagccaggacatcggcagcgacgtgatcctgctgtgcagcagcgaggagggcatccccaggcccacctacctgtgggagaagctggacaacaccctgaagctgccccccaccgccacccaggaccaggtgcagggcaccgtgaccatcaggaacatcagcgccctgagcagcggcctgtaccagtgcgtggccagcaacgccatcggcaccagcacctgcctgctggacctgcaggtgatcagcccccagcccaggaacatcggcctgatcgccggcgccatcggcaccggcgccgtgatcatcatcttctgcatcgccctgatcctgggcgccttcagcagggccggcagcctggtg

SEQ ID NO: 59 - DNA, 1056 bp, synthetic construct
  source 1..1056: mol_type = other DNA; note = Mutant of VSIG3 Variant 2
atgaccagccagaggagccccctggcccccctgctgctgctgagcctgcacggcgtggccgccagcctggaggtgagcgagagccccggcagcatccaggtggccaggggccagcccgccgtgctgccctgcaccttcaccaccagcgccgccctgatcaacctgaacgtgatctggatggtgacccccctgagcaacgccaaccagcccgagcaggtgatcctgtaccagggcggccagatgttcgacggcgcccccaggttccacggcagggtgggcttcaccggcaccatgcccgccaccaacgtgagcatcttcatcaacaacacccagctgagcgacaccggcacctaccagtgcctggtgaacaacctgcccgacatcggcggcaggaacatcggcgtgaccggcctgaccgtgctggtgccccccagcgccccccactgccagatccagggcagccaggacatcggcagcgacgtgatcctgctgtgcagcagcgaggagggcatccccaggcccacctacctgtgggagaagctggacaacaccctgaagctgccccccaccgccacccaggaccaggtgcagggcaccgtgaccatcaggaacatcagcgccctgagcagcggcctgtaccagtgcgtggccagcaacgccatcggcaccagcacctgcctgctggacctgcaggtgatcagcccccagcccaggaacatcggcctgatcgccggcgccatcggcaccggcgccgtgatcatcatcttctgcatcgccctgatcctgggcgccttcttctactggaggagcaagaacaaggaggaggaggaggaggagatccccaacgagatcagggaggacgacctgccccccaagtgcagcagcaccgccaacaggggcagcagcccccaggtgatgagcaggagcaacggcagcgtgagcaggaagcccaggcccccccacacccacagctacaccatcagccacgccaccctggagaggatcggcgccgtgcccgtgatggtgcccgcccagagcagggccggcagcctggtg

SEQ ID NO: 60 - protein, 350 aa, Homo sapiens
MRVGGAFHLLLVCLSPALLSAVRINGDGQEVLYLAEGDNVRLGCPYVLDPEDYGPNGLDIEWMQVNSDPAHHRENVFLSYQDKRINHGSLPHLQQRVRFAASDPSQYDASINLMNLQVSDTATYECRVKKTTMATRKVIVTVQARPAVPMCWTEGHMTYGNDVVLKCYASGGSQPLSYKWAKISGHHYPYRAGSYTSQHSYHSELSYQESFHSSINQGLNNGDLVLKDISRADDGLYQCTVANNVGYSVCVVEVKVSDSRRIGVIIGIVLGSLLALGCLAVGIWGLVCCCCGGSGAGGARGAFGYGNGGGVGGGACGDLASEIREDAVAPGCKASGRGSRVTHLLGYPTQ

SEQ ID NO: 61 - protein, 284 aa, synthetic construct
  source 1..284: note = Mutant of VSIG8 wt
MRVGGAFHLLLVCLSPALLSAVRINGDGQEVLYLAEGDNVRLGCPYVLDPEDYGPNGLDIEWMQVNSDPAHHRENVFLSYQDKRINHGSLPHLQQRVRFAASDPSQYDASINLMNLQVSDTATYECRVKKTTMATRKVIVTVQARPAVPMCWTEGHMTYGNDVVLKCYASGGSQPLSYKWAKISGHHYPYRAGSYTSQHSYHSELSYQESFHSSINQGLNNGDLVLKDISRADDGLYQCTVANNVGYSVCVVEVKVSDSRRIGVIIGIVLGSLLALGCLAVGIW

SEQ ID NO: 62 - protein, 285 aa, synthetic construct
  source 1..285: note = Mutant of VSIG8 wt
MRVGGAFHLLLVCLSPALLSAVRINGDGQEVLYLAEGDNVRLGCPYVLDPEDYGPNGLDIEWMQVNSDPAHHRENVFLSYQDKRINHGSLPHLQQRVRFAASDPSQYDASINLMNLQVSDTATYECRVKKTTMATRKVIVTVQARPAVPMCWTEGHMTYGNDVVLKCYASGGSQPLSYKWAKISGHHYPYRAGSYTSQHSYHSELSYQESFHSSINQGLNNGDLVLKDISRADDGLYQCTVANNVGYSVCVVEVKVSDSRRIGVIIGIVLGSLLALGCLAVGIWG

SEQ ID NO: 63 - protein, 286 aa, synthetic construct
  source 1..286: note = Mutant of VSIG8 wt
MRVGGAFHLLLVCLSPALLSAVRINGDGQEVLYLAEGDNVRLGCPYVLDPEDYGPNGLDIEWMQVNSDPAHHRENVFLSYQDKRINHGSLPHLQQRVRFAASDPSQYDASINLMNLQVSDTATYECRVKKTTMATRKVIVTVQARPAVPMCWTEGHMTYGNDVVLKCYASGGSQPLSYKWAKISGHHYPYRAGSYTSQHSYHSELSYQESFHSSINQGLNNGDLVLKDISRADDGLYQCTVANNVGYSVCVVEVKVSDSRRIGVIIGIVLGSLLALGCLAVGIWGL

SEQ ID NO: 64 - protein, 288 aa, synthetic construct
  source 1..288: note = Mutant of VSIG8 wt
MRVGGAFHLLLVCLSPALLSAVRINGDGQEVLYLAEGDNVRLGCPYVLDPEDYGPNGLDIEWMQVNSDPAHHRENVFLSYQDKRINHGSLPHLQQRVRFAASDPSQYDASINLMNLQVSDTATYECRVKKTTMATRKVIVTVQARPAVPMCWTEGHMTYGNDVVLKCYASGGSQPLSYKWAKISGHHYPYRAGSYTSQHSYHSELSYQESFHSSINQGLNNGDLVLKDISRADDGLYQCTVANNVGYSVCVVEVKVSDSRRIGVIIGIVLGSLLALGCLAVGIWGLVC

SEQ ID NO: 65 - protein, 290 aa, synthetic construct
  source 1..290: note = Mutant of VSIG8 wt
MRVGGAFHLLLVCLSPALLSAVRINGDGQEVLYLAEGDNVRLGCPYVLDPEDYGPNGLDIEWMQVNSDPAHHRENVFLSYQDKRINHGSLPHLQQRVRFAASDPSQYDASINLMNLQVSDTATYECRVKKTTMATRKVIVTVQARPAVPMCWTEGHMTYGNDVVLKCYASGGSQPLSYKWAKISGHHYPYRAGSYTSQHSYHSELSYQESFHSSINQGLNNGDLVLKDISRADDGLYQCTVANNVGYSVCVVEVKVSDSRRIGVIIGIVLGSLLALGCLAVGIWGLVCCC

SEQ ID NO: 66 - protein, 291 aa, synthetic construct
  source 1..291: note = Mutant of VSIG8 wt
MRVGGAFHLLLVCLSPALLSAVRINGDGQEVLYLAEGDNVRLGCPYVLDPEDYGPNGLDIEWMQVNSDPAHHRENVFLSYQDKRINHGSLPHLQQRVRFAASDPSQYDASINLMNLQVSDTATYECRVKKTTMATRKVIVTVQARPAVPMCWTEGHMTYGNDVVLKCYASGGSQPLSYKWAKISGHHYPYRAGSYTSQHSYHSELSYQESFHSSINQGLNNGDLVLKDISRADDGLYQCTVANNVGYSVCVVEVKVSDSRRIGVIIGIVLGSLLALGCLAVGIWGLVCCCC

SEQ ID NO: 67 - protein, 294 aa, synthetic construct
  source 1..294: note = Mutant of VSIG8 wt
MRVGGAFHLLLVCLSPALLSAVRINGDGQEVLYLAEGDNVRLGCPYVLDPEDYGPNGLDIEWMQVNSDPAHHRENVFLSYQDKRINHGSLPHLQQRVRFAASDPSQYDASINLMNLQVSDTATYECRVKKTTMATRKVIVTVQARPAVPMCWTEGHMTYGNDVVLKCYASGGSQPLSYKWAKISGHHYPYRAGSYTSQHSYHSELSYQESFHSSINQGLNNGDLVLKDISRADDGLYQCTVANNVGYSVCVVEVKVSDSRRIGVIIGIVLGSLLALGCLAVGIWGLVCCCCGGS

SEQ ID NO: 68 - protein, 301 aa, synthetic construct
  source 1..301: note = Mutant of VSIG8 wt
MRVGGAFHLLLVCLSPALLSAVRINGDGQEVLYLAEGDNVRLGCPYVLDPEDYGPNGLDIEWMQVNSDPAHHRENVFLSYQDKRINHGSLPHLQQRVRFAASDPSQYDASINLMNLQVSDTATYECRVKKTTMATRKVIVTVQARPAVPMCWTEGHMTYGNDVVLKCYASGGSQPLSYKWAKISGHHYPYRAGSYTSQHSYHSELSYQESFHSSINQGLNNGDLVLKDISRADDGLYQCTVANNVGYSVCVVEVKVSDSRRIGVIIGIVLGSLLALGCLAVGIWGLVCCCCGGSGAGGARG

SEQ ID NO: 69 - protein, 333 aa, synthetic construct
  source 1..333: note = Mutant of VSIG8 wt
MRVGGAFHLLLVCLSPALLSAVRINGDGQEVLYLAEGDNVRLGCPYVLDPEDYGPNGLDIEWMQVNSDPAHHRENVFLSYQDKRINHGSLPHLQQRVRFAASDPSQYDASINLMNLQVSDTATYECRVKKTTMATRKVIVTVQARPAVPMCWTEGHMTYGNDVVLKCYASGGSQPLSYKWAKISGHHYPYRAGSYTSQHSYHSELSYQESFHSSINQGLNNGDLVLKDISRADDGLYQCTVANNVGYSVCVVEVKVSDSRRIGVIIGIVLGSLLALGCLAVGIWGLVCCCCGGSGAGGARGAFGYGNGGGVGGGACGDLASEIREDAVAPGCK

SEQ ID NO: 70 - protein, 317 aa, synthetic construct
  source 1..317: note = Mutant of VSIG8 wt
MRVGGAFHLLLVCLSPALLSAVRINGDGQEVLYLAEGDNVRLGCPYVLDPEDYGPNGLDIEWMQVNSDPAHHRENVFLSYQDKRINHGSLPHLQQRVRFAASDPSQYDASINLMNLQVSDTATYECRVKKTTMATRKVIVTVQARPAVPMCWTEGHMTYGNDVVLKCYASGGSQPLSYKWAKISGHHYPYRAGSYTSQHSYHSELSYQESFHSSINQGLNNGDLVLKDISRADDGLYQCTVANNVGYSVCVVEVKVSDSRRIGVIIGIVLGSLLALGCLAVGIWGLVCCCCGGSGAGGARGAFGYGNGGGVGGGACG

SEQ ID NO: 71 - DNA, 1245 bp, Homo sapiens
  source 1..1245: mol_type = other DNA
atgagagttggaggagcattccaccttctactcgtgtgcctgagcccagcactgctgtctgctgtgcggatcaacggggatggacaggaggtcctgtacctggcagaaggtgataatgtgaggctgggctgcccctacgtcctggaccctgaggactatggtcccaatgggctggacatcgagtggatgcaggtcaactcagaccccgcccaccaccgagagaacgtgttccttagttaccaggacaagaggatcaaccatggcagccttccccatctgcagcagagggtccgctttgcagcctcagacccaagccagtacgatgcctccatcaacctcatgaacctgcaggtatctgatacagccacttatgagtgccgggtgaagaagaccaccatggccacccggaaggtcattgtcactgtccaagcacgacctgcagtgcccatgtgctggacagagggccacatgacatatggcaacgatgtggtgctgaagtgctatgccagtgggggctcccagcccctctcctacaagtgggccaagatcagtgggcaccattacccctatcgagctgggtcttacacctcccagcacagctaccactcagagctgtcctaccaggagtccttccacagctccataaaccaaggcctgaacaatggggacctggtgttgaaggatatctccagagcagatgatgggctgtatcagtgcacagtggccaacaacgtgggctacagtgtttgtgtggtggaggtgaaggtctcagactcccggcgtataggcgtgatcatcggcatcgtcctgggctctctgctcgcgctgggctgcctggccgtaggcatctgggggctcgtctgctgctgctgcgggggctccggggctggcggcgcccgcggtgccttcggctacggcaacggcggcggggtcggcggaggggcctgcggcgacttggctagtgagatcagagaggacgccgtggcgcccgggtgcaaggccagcgggcgcggcagccgcgtcacccacctcctggggtacccgacgcagaacgtcagccgctccctgcgccgcaagtacgcgcctcccccctgcggcggccccgaggacgtggccctggcgccctgcaccgccgccgccgcctgcgaagcgggcccctccccggtctacgtcaaggtcaagagcgcggagccggctgactgcgccgaggggccggtgcagtgcaagaacggcctcttggtgtga

SEQ ID NO: 72 - DNA, 852 bp, synthetic construct
  source 1..852: mol_type = other DNA; note = Mutant of VSIG8 wt
atgagggtgggcggcgccttccacctgctgctggtgtgcctgagccccgccctgctgagcgccgtgaggatcaacggcgacggccaggaggtgctgtacctggccgagggcgacaacgtgaggctgggctgcccctacgtgctggaccccgaggactacggccccaacggcctggacatcgagtggatgcaggtgaacagcgaccccgcccaccacagggagaacgtgttcctgagctaccaggacaagaggatcaaccacggcagcctgccccacctgcagcagagggtgaggttcgccgccagcgaccccagccagtacgacgccagcatcaacctgatgaacctgcaggtgagcgacaccgccacctacgagtgcagggtgaagaagaccaccatggccaccaggaaggtgatcgtgaccgtgcaggccaggcccgccgtgcccatgtgctggaccgagggccacatgacctacggcaacgacgtggtgctgaagtgctacgccagcggcggcagccagcccctgagctacaagtgggccaagatcagcggccaccactacccctacagggccggcagctacaccagccagcacagctaccacagcgagctgagctaccaggagagcttccacagcagcatcaaccagggcctgaacaacggcgacctggtgctgaaggacatcagcagggccgacgacggcctgtaccagtgcaccgtggccaacaacgtgggctacagcgtgtgcgtggtggaggtgaaggtgagcgacagcaggaggatcggcgtgatcatcggcatcgtgctgggcagcctgctggccctgggctgcctggccgtgggcatctgg

SEQ ID NO: 73 - DNA, 855 bp, synthetic construct
  source 1..855: mol_type = other DNA; note = Mutant of VSIG8 wt
atgagggtgggcggcgccttccacctgctgctggtgtgcctgagccccgccctgctgagcgccgtgaggatcaacggcgacggccaggaggtgctgtacctggccgagggcgacaacgtgaggctgggctgcccctacgtgctggaccccgaggactacggccccaacggcctggacatcgagtggatgcaggtgaacagcgaccccgcccaccacagggagaacgtgttcctgagctaccaggacaagaggatcaaccacggcagcctgccccacctgcagcagagggtgaggttcgccgccagcgaccccagccagtacgacgccagcatcaacctgatgaacctgcaggtgagcgacaccgccacctacgagtgcagggtgaagaagaccaccatggccaccaggaaggtgatcgtgaccgtgcaggccaggcccgccgtgcccatgtgctggaccgagggccacatgacctacggcaacgacgtggtgctgaagtgctacgccagcggcggcagccagcccctgagctacaagtgggccaagatcagcggccaccactacccctacagggccggcagctacaccagccagcacagctaccacagcgagctgagctaccaggagagcttccacagcagcatcaaccagggcctgaacaacggcgacctggtgctgaaggacatcagcagggccgacgacggcctgtaccagtgcaccgtggccaacaacgtgggctacagcgtgtgcgtggtggaggtgaaggtgagcgacagcaggaggatcggcgtgatcatcggcatcgtgctgggcagcctgctggccctgggctgcctggccgtgggcatctggggc

SEQ ID NO: 74 - DNA, 858 bp, synthetic construct
  source 1..858: mol_type = other DNA; note = Mutant of VSIG8 wt
atgagggtgggcggcgccttccacctgctgctggtgtgcctgagccccgccctgctgagcgccgtgaggatcaacggcgacggccaggaggtgctgtacctggccgagggcgacaacgtgaggctgggctgcccctacgtgctggaccccgaggactacggccccaacggcctggacatcgagtggatgcaggtgaacagcgaccccgcccaccacagggagaacgtgttcctgagctaccaggacaagaggatcaaccacggcagcctgccccacctgcagcagagggtgaggttcgccgccagcgaccccagccagtacgacgccagcatcaacctgatgaacctgcaggtgagcgacaccgccacctacgagtgcagggtgaagaagaccaccatggccaccaggaaggtgatcgtgaccgtgcaggccaggcccgccgtgcccatgtgctggaccgagggccacatgacctacggcaacgacgtggtgctgaagtgctacgccagcggcggcagccagcccctgagctacaagtgggccaagatcagcggccaccactacccctacagggccggcagctacaccagccagcacagctaccacagcgagctgagctaccaggagagcttccacagcagcatcaaccagggcctgaacaacggcgacctggtgctgaaggacatcagcagggccgacgacggcctgtaccagtgcaccgtggccaacaacgtgggctacagcgtgtgcgtggtggaggtgaaggtgagcgacagcaggaggatcggcgtgatcatcggcatcgtgctgggcagcctgctggccctgggctgcctggccgtgggcatctggggcctg

SEQ ID NO: 75 - protein, 864 aa, synthetic construct
  source 1..864: note = Mutant of VSIG8 wt
ATGAGGGTGGGCGGCGCCTTCCACCTGCTGCTGGTGTGCCTGAGCCCCGCCCTGCTGAGCGCCGTGAGGATCAACGGCGACGGCCAGGAGGTGCTGTACCTGGCCGAGGGCGACAACGTGAGGCTGGGCTGCCCCTACGTGCTGGACCCCGAGGACTACGGCCCCAACGGCCTGGACATCGAGTGGATGCAGGTGAACAGCGACCCCGCCCACCACAGGGAGAACGTGTTCCTGAGCTACCAGGACAAGAGGATCAACCACGGCAGCCTGCCCCACCTGCAGCAGAGGGTGAGGTTCGCCGCCAGCGACCCCAGCCAGTACGACGCCAGCATCAACCTGATGAACCTGCAGGTGAGCGACACCGCCACCTACGAGTGCAGGGTGAAGAAGACCACCATGGCCACCAGGAAGGTGATCGTGACCGTGCAGGCCAGGCCCGCCGTGCCCATGTGCTGGACCGAGGGCCACATGACCTACGGCAACGACGTGGTGCTGAAGTGCTACGCCAGCGGCGGCAGCCAGCCCCTGAGCTACAAGTGGGCCAAGATCAGCGGCCACCACTACCCCTACAGGGCCGGCAGCTACACCAGCCAGCACAGCTACCACAGCGAGCTGAGCTACCAGGAGAGCTTCCACAGCAGCATCAACCAGGGCCTGAACAACGGCGACCTGGTGCTGAAGGACATCAGCAGGGCCGACGACGGCCTGTACCAGTGCACCGTGGCCAACAACGTGGGCTACAGCGTGTGCGTGGTGGAGGTGAAGGTGAGCGACAGCAGGAGGATCGGCGTGATCATCGGCATCGTGCTGGGCAGCCTGCTGGCCCTGGGCTGCCTGGCCGTGGGCATCTGGGGCCTGGTGTGC

SEQ ID NO: 76 - DNA, 870 bp, synthetic construct
  source 1..870: mol_type = other DNA; note = Mutant of VSIG8 wt
atgagggtgggcggcgccttccacctgctgctggtgtgcctgagccccgccctgctgagcgccgtgaggatcaacggcgacggccaggaggtgctgtacctggccgagggcgacaacgtgaggctgggctgcccctacgtgctggaccccgaggactacggccccaacggcctggacatcgagtggatgcaggtgaacagcgaccccgcccaccacagggagaacgtgttcctgagctaccaggacaagaggatcaaccacggcagcctgccccacctgcagcagagggtgaggttcgccgccagcgaccccagccagtacgacgccagcatcaacctgatgaacctgcaggtgagcgacaccgccacctacgagtgcagggtgaagaagaccaccatggccaccaggaaggtgatcgtgaccgtgcaggccaggcccgccgtgcccatgtgctggaccgagggccacatgacctacggcaacgacgtggtgctgaagtgctacgccagcggcggcagccagcccctgagctacaagtgggccaagatcagcggccaccactacccctacagggccggcagctacaccagccagcacagctaccacagcgagctgagctaccaggagagcttccacagcagcatcaaccagggcctgaacaacggcgacctggtgctgaaggacatcagcagggccgacgacggcctgtaccagtgcaccgtggccaacaacgtgggctacagcgtgtgcgtggtggaggtgaaggtgagcgacagcaggaggatcggcgtgatcatcggcatcgtgctgggcagcctgctggccctgggctgcctggccgtgggcatctggggcctggtgtgctgctgc

SEQ ID NO: 77 - DNA, 873 bp, synthetic construct
  source 1..873: mol_type = other DNA; note = Mutant of VSIG8 wt
atgagggtgggcggcgccttccacctgctgctggtgtgcctgagccccgccctgctgagcgccgtgaggatcaacggcgacggccaggaggtgctgtacctggccgagggcgacaacgtgaggctgggctgcccctacgtgctggaccccgaggactacggccccaacggcctggacatcgagtggatgcaggtgaacagcgaccccgcccaccacagggagaacgtgttcctgagctaccaggacaagaggatcaaccacggcagcctgccccacctgcagcagagggtgaggttcgccgccagcgaccccagccagtacgacgccagcatcaacctgatgaacctgcaggtgagcgacaccgccacctacgagtgcagggtgaagaagaccaccatggccaccaggaaggtgatcgtgaccgtgcaggccaggcccgccgtgcccatgtgctggaccgagggccacatgacctacggcaacgacgtggtgctgaagtgctacgccagcggcggcagccagcccctgagctacaagtgggccaagatcagcggccaccactacccctacagggccggcagctacaccagccagcacagctaccacagcgagctgagctaccaggagagcttccacagcagcatcaaccagggcctgaacaacggcgacctggtgctgaaggacatcagcagggccgacgacggcctgtaccagtgcaccgtggccaacaacgtgggctacagcgtgtgcgtggtggaggtgaaggtgagcgacagcaggaggatcggcgtgatcatcggcatcgtgctgggcagcctgctggccctgggctgcctggccgtgggcatctggggcctggtgtgctgctgctgc

SEQ ID NO: 78 - DNA, 882 bp, synthetic construct
  source 1..882: mol_type = other DNA; note = Mutant of VSIG8 wt
atgagggtgggcggcgccttccacctgctgctggtgtgcctgagccccgccctgctgagcgccgtgaggatcaacggcgacggccaggaggtgctgtacctggccgagggcgacaacgtgaggctgggctgcccctacgtgctggaccccgaggactacggccccaacggcctggacatcgagtggatgcaggtgaacagcgaccccgcccaccacagggagaacgtgttcctgagctaccaggacaagaggatcaaccacggcagcctgccccacctgcagcagagggtgaggttcgccgccagcgaccccagccagtacgacgccagcatcaacctgatgaacctgcaggtgagcgacaccgccacctacgagtgcagggtgaagaagaccaccatggccaccaggaaggtgatcgtgaccgtgcaggccaggcccgccgtgcccatgtgctggaccgagggccacatgacctacggcaacgacgtggtgctgaagtgctacgccagcggcggcagccagcccctgagctacaagtgggccaagatcagcggccaccactacccctacagggccggcagctacaccagccagcacagctaccacagcgagctgagctaccaggagagcttccacagcagcatcaaccagggcctgaacaacggcgacctggtgctgaaggacatcagcagggccgacgacggcctgtaccagtgcaccgtggccaacaacgtgggctacagcgtgtgcgtggtggaggtgaaggtgagcgacagcaggaggatcggcgtgatcatcggcatcgtgctgggcagcctgctggccctgggctgcctggccgtgggcatctggggcctggtgtgctgctgctgcggcggcagc

SEQ ID NO: 79 - DNA, 903 bp, synthetic construct
  source 1..903: mol_type = other DNA; note = Mutant of VSIG8 wt
atgagggtgggcggcgccttccacctgctgctggtgtgcctgagccccgccctgctgagcgccgtgaggatcaacggcgacggccaggaggtgctgtacctggccgagggcgacaacgtgaggctgggctgcccctacgtgctggaccccgaggactacggccccaacggcctggacatcgagtggatgcaggtgaacagcgaccccgcccaccacagggagaacgtgttcctgagctaccaggacaagaggatcaaccacggcagcctgccccacctgcagcagagggtgaggttcgccgccagcgaccccagccagtacgacgccagcatcaacctgatgaacctgcaggtgagcgacaccgccacctacgagtgcagggtgaagaagaccaccatggccaccaggaaggtgatcgtgaccgtgcaggccaggcccgccgtgcccatgtgctggaccgagggccacatgacctacggcaacgacgtggtgctgaagtgctacgccagcggcggcagccagcccctgagctacaagtgggccaagatcagcggccaccactacccctacagggccggcagctacaccagccagcacagctaccacagcgagctgagctaccaggagagcttccacagcagcatcaaccagggcctgaacaacggcgacctggtgctgaaggacatcagcagggccgacgacggcctgtaccagtgcaccgtggccaacaacgtgggctacagcgtgtgcgtggtggaggtgaaggtgagcgacagcaggaggatcggcgtgatcatcggcatcgtgctgggcagcctgctggccctgggctgcctggccgtgggcatctggggcctggtgtgctgctgctgcggcggcagcggcgccggcggcgccaggggc

SEQ ID NO: 80 - DNA, 999 bp, synthetic construct
  source 1..999: mol_type = other DNA; note = Mutant of VSIG8 wt
atgagggtgggcggcgccttccacctgctgctggtgtgcctgagccccgccctgctgagcgccgtgaggatcaacggcgacggccaggaggtgctgtacctggccgagggcgacaacgtgaggctgggctgcccctacgtgctggaccccgaggactacggccccaacggcctggacatcgagtggatgcaggtgaacagcgaccccgcccaccacagggagaacgtgttcctgagctaccaggacaagaggatcaaccacggcagcctgccccacctgcagcagagggtgaggttcgccgccagcgaccccagccagtacgacgccagcatcaacctgatgaacctgcaggtgagcgacaccgccacctacgagtgcagggtgaagaagaccaccatggccaccaggaaggtgatcgtgaccgtgcaggccaggcccgccgtgcccatgtgctggaccgagggccacatgacctacggcaacgacgtggtgctgaagtgctacgccagcggcggcagccagcccctgagctacaagtgggccaagatcagcggccaccactacccctacagggccggcagctacaccagccagcacagctaccacagcgagctgagctaccaggagagcttccacagcagcatcaaccagggcctgaacaacggcgacctggtgctgaaggacatcagcagggccgacgacggcctgtaccagtgcaccgtggccaacaacgtgggctacagcgtgtgcgtggtggaggtgaaggtgagcgacagcaggaggatcggcgtgatcatcggcatcgtgctgggcagcctgctggccctgggctgcctggccgtgggcatctggggcctggtgtgctgctgctgcggcggcagcggcgccggcggcgccaggggcgccttcggctacggcaacggcggcggcgtgggcggcggcgcctgcggcgacctggccagcgagatcagggaggacgccgtggcccccggctgcaag

SEQ ID NO: 81 - DNA, 951 bp, synthetic construct
  source 1..951: mol_type = other DNA; note = Mutant of VSIG8 wt
atgagggtgggcggcgccttccacctgctgctggtgtgcctgagccccgccctgctgagcgccgtgaggatcaacggcgacggccaggaggtgctgtacctggccgagggcgacaacgtgaggctgggctgcccctacgtgctggaccccgaggactacggccccaacggcctggacatcgagtggatgcaggtgaacagcgaccccgcccaccacagggagaacgtgttcctgagctaccaggacaagaggatcaaccacggcagcctgccccacctgcagcagagggtgaggttcgccgccagcgaccccagccagtacgacgccagcatcaacctgatgaacctgcaggtgagcgacaccgccacctacgagtgcagggtgaagaagaccaccatggccaccaggaaggtgatcgtgaccgtgcaggccaggcccgccgtgcccatgtgctggaccgagggccacatgacctacggcaacgacgtggtgctgaagtgctacgccagcggcggcagccagcccctgagctacaagtgggccaagatcagcggccaccactacccctacagggccggcagctacaccagccagcacagctaccacagcgagctgagctaccaggagagcttccacagcagcatcaaccagggcctgaacaacggcgacctggtgctgaaggacatcagcagggccgacgacggcctgtaccagtgcaccgtggccaacaacgtgggctacagcgtgtgcgtggtggaggtgaaggtgagcgacagcaggaggatcggcgtgatcatcggcatcgtgctgggcagcctgctggccctgggctgcctggccgtgggcatctggggcctggtgtgctgctgctgcggcggcagcggcgccggcggcgccaggggcgccttcggctacggcaacggcggcggcgtgggcggcggcgcctgcggc

SEQ ID NO: 82 - DNA, 939 bp, synthetic construct
  source 1..939: mol_type = other DNA; note = Mutant of VSIG3 Variant 6
atgaccagccagaggagccccctggcccccctgctgctgctgagcctgcacggcgtggccgccagcctggaggtgagcgagagccccggcagcatccaggtggccaggggccagcccgccgtgctgccctgcaccttcaccaccagcgccgccctgatcaacctgaacgtgatctggatggtgacccccctgagcaacgccaaccagcccgagcaggtgatcctgtaccagggcggccagatgttcgacggcgcccccaggttccacggcagggtgggcttcaccggcaccatgcccgccaccaacgtgagcatcttcatcaacaacacccagctgagcgacaccggcacctaccagtgcctggtgaacaacctgcccgacatcggcggcaggaacatcggcgtgaccggcctgaccgtgctggtgccccccagcgccccccactgccagatccagggcagccaggacatcggcagcgacgtgatcctgctgtgcagcagcgaggagggcatccccaggcccacctacctgtgggagaagctggacaacaccctgaagctgccccccaccgccacccaggaccaggtgcagggcaccgtgaccatcaggaacatcagcgccctgagcagcgcccagcccaggaacatcggcctgatcgccggcgccatcggcaccggcgccgtgatcatcatcttctgcatcgccctgatcctgggcgccttcttctactggaggagcaagaacaaggaggaggaggaggaggagatccccaacgagatcagggaggacgacctgccccccaagtgcagcagcgccaaggccttccacaccgagatcagcagcagcgacaacaacaccctgaccagcagcaacgcctacaacagcaggtactggagcaacaaccccaaggtgcacaggaacaccgagagcgtgagccac